(19) United States
(12) Patent Application Publication
(10) Pub. No.: US 2023/0279385 A1
(43) Pub. Date: Sep. 7, 2023

(54) Sequence-Specific Targeted Transposition and Selection and Sorting of Nucleic Acids

(71) Applicants: Illumina, Inc., San Diego, CA (US); Illumina Cambridge Limited, Cambridge (GB)

(72) Inventors: Frank J. STEEMERS, San Diego, CA (US); Jonathan Mark BOUTELL, Hertfordshire (GB); Pietro GATTI LAFRANCONI, Cambridge (GB); Oliver Jon MILLER, Nr Saffron Walden (GB); Emma BELL, Cambridge (GB); Sebastien Georg Gabriel RICOULT, Nr Saffron Walden (GB); Niall Anthony GORMLEY, Cambridge (GB); Kim SCHNEIDER, Cambridge (GB)

(73) Assignees: Illumina, Inc., San Diego, CA (US); Illumina Cambridge Limited, Cambridge (GB)

(21) Appl. No.: 18/169,970

(22) Filed: Feb. 16, 2023

Related U.S. Application Data
(63) Continuation of application No. PCT/US21/46292, filed on Aug. 17, 2021.
(60) Provisional application No. 63/066,905, filed on Aug. 18, 2020.
(60) Provisional application No. 63/066,906, filed on Aug. 18, 2020.
(60) Provisional application No. 63/162,775, filed on Mar. 18, 2021.
(60) Provisional application No. 63/163,381, filed on Mar. 19, 2021.
(60) Provisional application No. 63/168,753, filed on Mar. 31, 2021.
(60) Provisional application No. 63/228,344, filed on Aug. 2, 2021.

(51) Int. Cl. C12N 15/10; C12N 15/11; C12N 9/22; C12N 9/12

(52) U.S. Cl. CPC C12N 15/1065; C12N 15/11; C12N 9/22; C12N 9/1241; C12N 2310/20; C12N 2800/80; C12N 2800/90

(57) ABSTRACT

A variety of different types of targeted transposome complexes are described herein that may be used to mediate sequence-specific targeted transposition of nucleic acids. Also described herein is a method of characterizing desired samples in a mixed pool of samples comprising both desired samples and unwanted samples comprising, to produce sequencing data from double-stranded nucleic acid, initially sequencing a library comprising a plurality of nucleic acid samples from a mixed pool, wherein each nucleic acid library comprises nucleic acids from a single sample and a unique sample barcode to distinguish the nucleic acids from the single sample from the nucleic acids from other samples in the library; analyzing the sequencing data and identifying unique sample barcodes associated with sequencing data from desired samples; performing a selection step on the library comprising enriching nucleic acid samples from desired samples and/or depleting nucleic acid samples from unwanted samples; and resequencing the nucleic acid library.

### CROSS-REFERENCE TO RELATED APPLICATIONS

This application is a bypass continuation of PCT/US2021/046292, filed Aug. 17, 2021, which claims the benefit of priority of U.S. Provisional Application Nos. 63/066,905 and 63/066,906, each filed Aug. 18, 2020; U.S. 63/162,775, filed Mar. 18, 2021; U.S. 63/163,381, filed Mar. 19, 2021; U.S. 63/168,753, filed Mar. 31, 2021; and U.S. 63/228,344, filed Aug. 2, 2021; the contents of which are each incorporated by reference herein in their entireties for any purpose.

### SEQUENCE LISTING

The present application is filed with a Sequence Listing in electronic XML format. The Sequence Listing is provided as a file entitled “2023-04-21-01243-0020-00US-ST26” created on Apr. 21, 2023, which is 11,946 bytes in size. The information in the electronic format of the sequence listing is incorporated herein by reference in its entirety.

### DESCRIPTION

### Field

This disclosure related to sequence-specific targeted transposition of nucleic acids. Targeted transposome complexes may be used to mediate sequence-specific targeted transposition. This disclosure relates to a method comprising initial sequencing, selection, and resequencing for evaluating desired samples. As described herein, initial sequencing can identify samples of interest in a pool of mixed samples, and unwanted samples can then be depleted, or desired samples can be enriched based on unique sample barcodes. Resequencing can then be performed on desired samples.

### Background

Library generation of selected regions of a target nucleic may desired for a number of different applications. For example, the ability to make libraries from selected regions of genomic DNA is desired where platform outputs are limiting (e.g. PacBio, ONT, or iSeq). Also, libraries for selected regions of genomic DNA are advantageous when very high coverage is required, such as screening for rare somatic mutation in liquid biopsy samples.

Current methods to achieve libraries from selected regions of genomic DNA include oligonucleotide hybridization-based enrichment kits (e.g., TruSeq Exome, Nextera Flex for Enrichment). In addition, CRISPR-based systems for generating such libraries have been published recently. In particular, the CRISPR-based systems have been used to pull out regions of 10's-100's of kilobases, which are suitable for long-read technologies such as PacBio and ONT.

The disclosure describes new way of targeted library preparation of desired regions of genomic DNA. These methods combine different targeting technologies with transposomes in a number of unique ways. Further, this disclosure describes means of targeted library preparation from cell-free DNA (cfDNA) without requiring removal of histones before tagmentation.

This disclosure also describes single-cell analysis methods that can be used to resolve cellular differences that are difficult to determine when studying bulk population of cells. Characterization of rare cells can be important for a number of uses, such as in oncology (liquid or tumor biopsy, minimum residual disease or early disease detection, tumor evolution, or tumor resistance), immunology (immune or T cell receptor repertoire), and metagenomics (uncultivatable organism genome assembly). FIG. 1 provides some representative examples of metagenomic and oncology samples that may be of interest, wherein rare cells are of high interest. Current methods in single-cell sequencing enable cell-resolved ‘omic’ characterization of millions of single-cells in parallel, such as to study genomic, transcriptomic, or epigenomic features of individual cells.

However, comprehensive sequencing-based characterization of rare cells in a population is costly and challenging in the absence of selection of desired samples. Furthermore, cell sorting-based enrichment methods are limited based on the availability of partitionable cell features. For example, FACS can enrich for certain cell size, morphology, and surface protein expression, but other characteristics may not be partitionable by FACS. It would be of great utility to enrich cells based on a particular ‘omic’ features (e.g. enrichment based on species, cell type, or presence of variant). These features may be known a priori (based on the state of the art) or de novo (determined by an initial sequencing analysis). It would also be of great value to perform follow-up, comprehensive/orthogonal ‘omic’ characterization by resequencing of samples from single cells identified to be of interest after initial sequencing.

Disclosed herein are methodologies for the selection, enrichment, and sequencing-based characterization of an individual cell's DNA library from a “single-cell sequencing library” or “sc library” consisting of a plurality of cellular DNA libraries comprising libraries generated from different single cells. Initial sequencing of the sc-library (i.e., sequencing of all DNA libraries from individual cells) can be performed, and bioinformatic analysis can be used to sort the individual cells with respect to a particular ‘-omic’ feature of interest. Using this method, libraries generated from different individual cells are identified by unique cellular DNA barcodes (UBCs). The ‘-omic’ feature used for sorting may define cell type (e.g. expression, epigenetic pattern, or immune gene recombination), species type (e.g. using 16s, 18s, or ITS rRNA/rDNA sequencing from bacteria) or disease state/risk (e.g. cancer-significant germline or somatic variants) with a relatively small, targeted sequencing panel. In other words, the footprint of the initial sequencing may be small, and resequencing can be more comprehensive but focused on cells of interest. One skilled in the art can thus query millions or billions of cells for an exemplary feature using a single initial sequencing run to sort samples into desired and unwanted samples followed by a targeted resequencing of desired samples.

Alternatively, the initial sequencing run may be used to identify de novo exemplary ‘omic’ cellular feature(s) for follow-up analysis. For example, the initial sequencing run may identify a new cellular feature that can then be used for sorting.

The enrichment or depletion in the present method may be performed by known nucleic acid target enrichment methods (e.g. hybrid capture, unique sample barcode-specific amplification, or CRISPR digestion). Individual cellular DNA from cells of interest can then be resequenced and characterized in isolation from the full sc-library. Thus, the present method can allow more comprehensive and/or orthogonal resequencing and analysis after an initial sequencing run that acts to sort the cells.

### SUMMARY

This disclosure describes a number of different targeted transposome complexes, which comprise one or more element that direct the transposome complex to bind one or more nucleic acid sequences of interest in a target nucleic acid. Also described herein are a number of methods that use these targeted transposome complexes.

In accordance with the description, a method of characterizing desired samples in a mixed pool of samples comprising both desired samples and unwanted samples is also described.

Embodiment 1. A targeted transposome complex comprising a transposase, a first transposon comprising a 3′ transposon end sequence; a 5′ adaptor sequence; and a targeting oligonucleotide coated with a recombinase, wherein the targeting oligonucleotide can bind to one or more nucleic acid sequences of interest; and a second transposon comprising a 5′ transposon end sequence, wherein the 5′ transposon end sequence is complementary to the 3′ transposon end sequence.

Embodiment 2. The transposome complex of embodiment 1, wherein the sequence of the targeting oligonucleotide is fully or partially complementary with the one or more nucleic acid sequences of interest.

Embodiment 3. The transposome complex of any one of embodiments 1 or 2, wherein one or more targeting oligonucleotide are linked to the 5′ end of the adaptor sequence.

Embodiment 4. The transposome complex of any one of embodiments 1-3, wherein one or more targeting oligonucleotide are linked directly to the 5′ end of the adaptor sequence.

Embodiment 5. The transposome complex of any one of embodiments 1-4, wherein one or more targeting oligonucleotide are linked via a linker to the 5′ end of the adaptor sequence.

Embodiment 6. The transposome complex of embodiment 1-5, wherein the linker is an oligonucleotide linker.

Embodiment 7. The transposome complex of embodiment 1-6, wherein the linker is a non-oligonucleotide linker.

Embodiment 8. The transposome complex of embodiment 1-7, wherein the 5′ end of the adaptor sequence and the targeting oligonucleotide are both biotinylated and linked via streptavidin.

Embodiment 9. The transposome complex of any one of embodiments 1-8, wherein the adaptor sequence comprises a primer sequence, an index tag sequence, a capture sequence, a barcode sequence, a cleavage sequence, or a sequencing-related sequence, or a combination thereof.

Embodiment 10. The transposome complex of embodiment 1-9, wherein the adaptor sequence comprises a P5 or P7 sequence.

Embodiment 11. The transposome complex of any one of embodiments 1-10, wherein the recombinase is UVSX, Rec233, or RecA.

Embodiment 12. The transposome complex of any one of embodiment 1-11, wherein the transposome complex is in solution.

Embodiment 13. The transposome complex of any one of embodiment 1-12, wherein the transposome complex is immobilized to a solid support.

Embodiment 14. The transposome complex of embodiment 1-13, wherein the solid support is a bead.

Embodiment 15. A kit or composition comprising a first transposome complex of any one of embodiments 1-14 that is a targeted transposome complex, and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence.

Embodiment 16. A kit or composition comprising two transposome complexes of any one of embodiments 1-14 that are each a targeted transposome complex, wherein the two targeted transposome complexes comprises different targeting oligonucleotides.

Embodiment 17. A method of targeted generation of 5′ tagged fragments of a target nucleic acid comprising combining a sample comprising a double-stranded nucleic acid and a transposome complexes of any one of embodiments 1-14 that is a targeted transposome complex; initiating strand invasion of the nucleic acid by the recombinase; and fragmenting the nucleic acid into a plurality of fragments by the transposase, by joining the 3′ end of the first transposon to the 5′ ends of the fragments to produce a plurality of 5′ tagged fragments.

Embodiment 18. A method of generating a library of tagged nucleic acid fragments comprising combining a sample comprising a double-stranded nucleic acid, a first transposome complex of any one of embodiments 1-14 that is a targeted transposome complex, and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence; initiating strand invasion of the nucleic acid by the recombinase; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

Embodiment 19. A method of generating a library of tagged nucleic acid fragments comprising combining a sample comprising a double-stranded nucleic acid, a first transposome complex of any one of embodiments 1-14 that is a targeted transposome complex, and a second transposome complex of any one of embodiments 1-14 that is a targeted transposome complex; initiating strand invasion of the nucleic acid by the recombinase; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

Embodiment 20. The method of any one of embodiments 17-19 or the kit or composition of embodiment 15 or embodiment 16, wherein the 5′ adaptor sequences comprised in the first transposome complex and the second transposome complex are different.

Embodiment 21. The method of embodiment 19, wherein the targeting oligonucleotide comprised in the first transposome complex that is a targeted transposome complex and the second transposome complex that is a targeted transposome complex are different.

Embodiment 22. The method of embodiment 21, wherein the targeting oligonucleotide of the first transposome complex that is a targeted transposome complex and the second transposome complex that is a targeted transposome complex bind to different sequences of interest in a given region of interest in a target nucleic acid.

Embodiment 23. The method of embodiment 22, wherein the targeting oligonucleotide of the first transposome complex that is a targeted transposome complex and the second transposome complex that is a targeted transposome complex bind to opposite strands of the double-stranded nucleic acid.

Embodiment 24. The method of any one of embodiments 17-23, wherein initiating strand invasion of the nucleic acid by the recombinase is performed in the presence of a recombinase loading factor; optionally wherein the recombinase loading factor is removed or inactivated before fragmenting.

Embodiment 25. The method of any one of embodiments 17-24, wherein initiating strand invasion occurs via displacement loop formation.

Embodiment 26. The method of any one of embodiments 17-25, wherein strand invasion is initiated within 40, 30, 20, 15, 10, or 5 bases of the binding site of the targeting oligonucleotide to the one or more sequences of interest.

Embodiment 27. The method of any one of embodiments 17-26, wherein the temperature used for initiating strand invasion is different from the optimum temperature for fragmenting by the transposase.

Embodiment 28. The method of embodiment 27, wherein the temperature used for initiating strand invasion is below the optimum temperature for fragmenting by the transposase.

Embodiment 29. The method of embodiment 28, wherein initiating strand invasion is performed at 27° C. to 47° C.

Embodiment 30. The method of embodiment 29, wherein initiating strand invasion is performed at 32° C. to 42° C.

Embodiment 31. The method of embodiment 30, wherein initiating strand invasion is performed at 37° C.

Embodiment 32. The method of any one of embodiments 28, wherein the fragmenting is performed at 45° C. to 65° C.

Embodiment 33. The method of any one of embodiments 32, wherein the fragmenting is performed at 50° C. to 60° C.

Embodiment 34. The method of any one of embodiments 33, wherein the fragmenting is performed at 55° C.

Embodiment 35. The method of any one of embodiments 17-34, wherein a cofactor for the transposase is added to the transposome complexes after initiating invasion and before fragmenting.

Embodiment 36. The method of embodiment 35, wherein the cofactor is Mg⁺⁺.

Embodiment 37. The method of embodiment 36, wherein the Mg⁺⁺ concentration is 10 mM to 18 mM.

Embodiment 38. The method of any one of embodiments 17-37, wherein the fragmenting occurs within 40, 30, 20, 15, 10, or 5 bases of the one or more sequences of interest in a nucleic acid sequence bound by the targeting oligonucleotide.

Embodiment 39. The method of any one of embodiments 17-38, further comprising treating the plurality of 5′ tagged fragments with a polymerase and a ligase to extend and ligate the strands to produce fully double-stranded tagged fragments.

Embodiment 40. The method of any one of embodiments 17-39, further comprising sequencing one or more of the 5′ tagged fragments or fully double-stranded tagged fragments.

Embodiment 41. A method of preserving contiguity information when sequencing a target nucleic acid comprising producing tagged fragments of the target nucleic acid according to the method of any one of embodiments 17-40; sequencing the 5′ tagged fragments or fully double-stranded tagged fragments to provide sequences of the fragments; grouping sequences of fragments that comprise the sequence of the same targeting oligonucleotide; and determining that a group of sequences were in proximity within the target nucleic acid if they comprise the sequence of the same targeting oligonucleotide.

Embodiment 42. A method of preserving contiguity information when sequencing a target nucleic acid comprising producing tagged fragments of the target nucleic acid according to the method of any one of embodiments 17-40, wherein one or more adapter sequence comprises a unique molecular identifier (UMI) associated with a single targeting oligonucleotide sequence; sequencing the 5′ tagged fragments or fully double-stranded tagged fragments to provide sequences of the fragments; grouping sequences of fragments that comprise the sequence of the same UMI; and determining that a group of sequences were in proximity within the target nucleic acid if they comprise the sequence of the same UMI.

Embodiment 43. A method of targeted generation of 5′ tagged fragments of nucleic acid comprising hybridizing one or more targeting oligonucleotides to a sample comprising single-stranded nucleic acid, wherein the one or more targeting oligonucleotides can each bind to a sequence of interest in the nucleic acid; applying a transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence; and fragmenting the nucleic acid into a plurality of fragments by the transposase, by joining the 3′ end of the first transposon to the 5′ ends of the fragments to produce a plurality of 5′ tagged fragments.

Embodiment 44. The method of embodiment 43, wherein double-stranded DNA is denatured to generate the single-stranded DNA.

Embodiment 45. The method of any one of embodiments 43-44, wherein hybridizing a targeting oligonucleotide to a sample comprising single-stranded nucleic acid generates a region of double-stranded nucleic acid that can be fragmented.

Embodiment 46. The method of any one of embodiments 43-45, wherein two or more targeting oligonucleotides with different sequences are hybridized.

Embodiment 47. The method of any one of embodiments 43-45, wherein multiple copies of a single targeting oligonucleotide are hybridized.

Embodiment 48. The method of embodiment 47, wherein the single targeting oligonucleotide is long enough to allow binding of two transposome complexes to the double-stranded nucleic acid generated by hybridizing the single targeting oligonucleotide to the sample comprising single-stranded nucleic acid.

Embodiment 49. The method of embodiment 47 or embodiment 48, wherein the single targeting oligonucleotide comprises 80, 90, 100, 110, 120, 130, 140, 150, 160, 170, 180, 190, or 200 base pairs.

Embodiment 50. The method of any one of embodiments 43-49, wherein the fragmenting occurs within the one or more sequences of interest in a nucleic acid sequence bound by the one or more targeting oligonucleotide.

Embodiment 51. The method of any one of embodiments 43-50, further comprising treating the plurality of 5′ tagged fragments with a polymerase and a ligase to extend and ligate the strands to produce fully double-stranded tagged fragments.

Embodiment 52. The method of any one of embodiments 43-51, further comprising sequencing one or more of the 5′ tagged fragments or fully double-stranded tagged fragments.

Embodiment 53. A targeted transposome complex comprising a transposase; a first transposon comprising a 3′ transposon end sequence, a 5′ adaptor sequence, and a catalytically inactive endonuclease associated with a guide RNA, wherein the guide RNA can direct endonuclease binding to one or more nucleic acid sequences of interest; and a second transposon comprising the complement of the transposon end sequence.

Embodiment 54. The transposome complex of embodiment 53, wherein the catalytically inactive endonuclease binds nucleic acid but does not initiate cleavage.

Embodiment 55. The transposome complex of embodiment 53 or embodiment 54, wherein the guide RNA is a single guide RNA.

Embodiment 56. The transposome complex of any one of embodiments 53-55, wherein the catalytically inactive endonuclease is associated with the transposase.

Embodiment 57. The transposome complex of embodiment 56, wherein the catalytically inactive endonuclease is linked to the transposase.

Embodiment 58. The transposome complex of any one of embodiments 53-57, wherein the transposase and the catalytically inactive endonuclease are comprised in a CRISPR-associated transposase.

Embodiment 59. The transposome complex of embodiment 58, wherein the CRISPR-associated transposase is from cyanobacteria Scytonema hofmanni (ShCAST), optionally wherein:
a. ShCAST is coupled to a guide RNA, optionally wherein at least one of the gRNA and the transposase is biotinylated, and wherein at least one of the gRNA and transposase that is biotinylated is capable of coupling to a streptavidin-coated bead;
b. ShCAST comprises Cas12K;
c. the transposase comprises Tn5 or a Tn7-like transposase, optionally wherein the first transposon comprises at least one of a P5 adapter and a P7 adapter.

Embodiment 60. The transposome complex of embodiment 57, wherein the catalytically inactive endonuclease is linked to the 5′ end of the transposase.

Embodiment 61. The transposome complex of embodiment 57, wherein the catalytically inactive endonuclease is linked to the 3′ end of the transposase.

Embodiment 62. The transposome complex of embodiment 57, wherein the transposase is linked to the 5′ end of the catalytically inactive endonuclease.

Embodiment 63. The transposome complex of embodiment 57, wherein the transposase is linked to the 3′ end of the catalytically inactive endonuclease.

Embodiment 64. The transposome complex of any one of embodiments 53-63, wherein the catalytically inactive endonuclease and transposase are comprised in a fusion protein.

Embodiment 65. The transposome complex of embodiment 64, wherein the catalytically inactive and transposase are linked via a linker.

Embodiment 66. The transposome complex of any one of embodiments 53-56, wherein the catalytically inactive endonuclease and transposase are comprised in separate proteins.

Embodiment 67. The transposome complex of embodiment 66, wherein the separate catalytically inactive endonuclease and transposase can associate together via pairing of binding partners, wherein a first binding partner is bound to the catalytically inactive endonuclease and a second binding partner is bound to the transposase.

Embodiment 68. The transposome complex of embodiment 67, wherein the binding partners are biotin and streptavidin/avidin.

Embodiment 69. The transposome complex of any one of embodiments 55-68, wherein the single guide RNA is comprised in an oligonucleotide comprising the first and/or second transposon.

Embodiment 70. The transposome complex of embodiment 69, wherein the oligonucleotide comprises a 5′ single guide RNA and a 3′ first and/or second transposon.

Embodiment 71. The transposome complex of any one of embodiments 53-70, wherein the single guide RNA comprises less than 20 nucleotides.

Embodiment 72. The transposome complex of embodiment 71, wherein the single guide RNA sequence comprises 15, 16, 17, 18, or 19 nucleotides.

Embodiment 73. The transposome complex of any one of embodiments 53-72, wherein the single guide RNA comprises a hairpin secondary structure.

Embodiment 74. The transposome complex of any one of embodiments 53-73, wherein the catalytically inactive endonuclease is a Cas9 protein.

Embodiment 75. The transposome complex of embodiment 74, wherein the Cas9 protein is a Streptococcus canis Cas9.

Embodiment 76. The transposome complex of any one of embodiments 53-75, wherein the Streptococcus canis Cas9 has minimal sequence constraint.

Embodiment 77. A targeted transposome complex comprising a transposase, a first transposon comprising a 3′ transposon end sequence; a 5′ adaptor sequence; and a zinc finger DNA-binding domain, wherein the zinc finger DNA-binding domain can bind to one or more nucleic acid sequences of interest; and a second transposon comprising the complement of the transposon end sequence.

Embodiment 78. The targeted transposome complex of embodiment 77, wherein the zinc finger DNA-binding domain is comprised in a zinc finger nuclease.

Embodiment 79. The targeted transposome complex of embodiment 78, wherein the zinc finger nuclease is catalytically inactive.

Embodiment 80. The targeted transposome complex of any one of embodiments 77-79, wherein the one or more nucleic acid sequences of interest are comprised in DNA associated with histones.

Embodiment 81. The targeted transposome complex of embodiment 80, wherein the DNA associated with histones is cell-free DNA.

Embodiment 82. The targeted transposome complex of any one of embodiments 77-81, wherein the first transposon comprises an affinity element.

Embodiment 83. The targeted transposome complex of embodiment 82, wherein the affinity element is attached to the 5′ end of the first transposon.

Embodiment 84. The targeted transposome complex of any one of embodiments 82-83, wherein the first transposon comprises a linker.

Embodiment 85. The targeted transposome complex of embodiment 84, wherein the linker has a first end attached to the 5′ end of the first transposon and a second end attached to an affinity element.

Embodiment 86. The targeted transposome complex of any one of embodiments 77-85, wherein the second transposon comprises an affinity element.

Embodiment 87. The targeted transposome complex of embodiment 86, wherein the affinity element is attached to the 3′ end of the second transposon.

Embodiment 88. The targeted transposome complex of any one of embodiments 82-85, wherein the second transposon comprises a linker.

Embodiment 89. The targeted transposome complex of embodiment 88, wherein the linker has a first end attached to the 3′ end of the second transposon and a second end attached to an affinity element.

Embodiment 90. The targeted transposome complex of any one of embodiments 82-89, wherein the affinity element is biotin.

Embodiment 91. The targeted transposome complex of embodiment 77-90, wherein the complex comprises a zinc finger DNA-binding domain array.

Embodiment 92. The transposome complex of embodiment 77-91, wherein the zinc finger DNA-binding domain is associated with the transposase.

Embodiment 93. The transposome complex of embodiment 92, wherein the zinc finger DNA-binding domain is linked to the transposase.

Embodiment 94. The transposome complex of embodiment 93, wherein the zinc finger DNA-binding domain is linked to the 5′ end of the transposase.

Embodiment 95. The transposome complex of embodiment 93, wherein the zinc finger DNA-binding domain is linked to the 3′ end of the transposase.

Embodiment 96. The transposome complex of embodiment 94 or 95, wherein the transposase is linked to the 5′ end of the zinc finger DNA-binding domain.

Embodiment 97. The transposome complex of embodiment 94 or 95, wherein the transposase is linked to the 3′ end of the zinc finger DNA-binding domain.

Embodiment 98. The transposome complex of any one of embodiments 77-97, wherein the zinc finger DNA-binding domain and transposase are comprised in a fusion protein.

Embodiment 99. The transposome complex of any one of embodiments 77-98, wherein the zinc finger DNA-binding domain and transposase are linked via a linker.

Embodiment 100. The transposome complex of any one of embodiments 77-92, wherein the zinc finger DNA-binding domain and transposase are comprised in separate proteins.

Embodiment 101. The transposome complex of embodiment 100, wherein the separate zinc finger DNA-binding domain and transposase can associate together via pairing of binding partners, wherein a first binding partner is bound to the catalytically inactive endonuclease and a second binding partner is bound to the transposase.

Embodiment 102. The transposome complex of embodiment 101, wherein the binding partners are (i) biotin and (ii) streptavidin or avidin.

Embodiment 103. The transposome complex of any one of embodiments 53-102, wherein the adaptor sequence comprises a primer sequence, an index tag sequence, a capture sequence, a barcode sequence, a cleavage sequence, or a sequencing-related sequence, or a combination thereof.

Embodiment 104. The transposome complex of embodiments 53-103, wherein the adaptor sequence comprises a P5 or P7 sequence.

Embodiment 105. The transposome complex of any one of embodiments 53-104, wherein the transposome complex is in solution.

Embodiment 106. The transposome complex of any one of embodiments 53-105, wherein the transposome complex is immobilized to a solid support.

Embodiment 107. The transposome complex of embodiment 106, wherein the solid support is a bead.

Embodiment 108. A kit or composition comprising a first transposome complex of any one of embodiments 53-107 that is a targeted transposome complex, and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence.

Embodiment 109. A kit or composition of embodiment 108, comprising two transposome complexes of any one of embodiments 53-107 that are each a targeted transposome complex, wherein the two targeted transposome complexes comprise different guide RNAs.

Embodiment 110. A kit or composition comprising two transposome complexes of any one of embodiments 108 or 109 that are each a targeted transposome complex, wherein the two targeted transposome complexes comprise different zinc finger DNA-binding domains.

Embodiment 111. A method of targeted generation of 5′ tagged fragments of a target nucleic acid comprising combining a sample comprising a double-stranded nucleic acid and a transposome complexes of any one of embodiments 53-107 that is a targeted transposome complex; and fragmenting the nucleic acid into a plurality of fragments by the transposase, by joining the 3′ end of the first transposon to the 5′ ends of the fragments to produce a plurality of 5′ tagged fragments.

Embodiment 112. A method of generating a library of tagged nucleic acid fragments comprising combining a sample comprising a double-stranded nucleic acid, a first transposome complex of any one of embodiments 53-107 that is a targeted transposome complex, and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

Embodiment 113. A method of generating a library of tagged nucleic acid fragments comprising combining a sample comprising a double-stranded nucleic acid, a first transposome complex of any one of embodiments 53-107 that is a targeted transposome complex, and a second transposome complex of any one of embodiments 53-107 that is a targeted transposome complex; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

Embodiment 114. The method of any one of embodiments 111-113, wherein the first and/or second targeted transposome complex comprise a zinc finger DNA-binding domain.

Embodiment 115. The method of embodiment 114, wherein the zinc finger DNA-binding domain is comprised in a zinc finger nuclease.

Embodiment 116. The method of embodiment 115, wherein the zinc finger nuclease is catalytically inactive.

Embodiment 117. The method of any one of embodiments 111-116, wherein the first transposon comprised in the targeted transposome complex comprises an affinity element.

Embodiment 118. The method of embodiment 117, wherein the affinity element is attached to the 5′ end of the first transposon.

Embodiment 119. The method of any one of embodiments 118, wherein the first transposon comprised in the targeted transposome complex comprises a linker.

Embodiment 120. The method of embodiment 119, wherein the linker has a first end attached to the 5′ end of the first transposon and a second end attached to an affinity element.

Embodiment 121. The method of any one of embodiments 111-120, wherein the second transposon comprises an affinity element.

Embodiment 122. The method of embodiment 121, wherein the affinity element is attached to the 3′ end of the second transposon.

Embodiment 123. The method of embodiment 121, wherein the second transposon comprises a linker.

Embodiment 124. The method of embodiment 123, wherein the linker has a first end attached to the 3′ end of the second transposon and a second end attached to an affinity element.

Embodiment 125. The method of any one of embodiments 117-124, wherein the affinity element is biotin.

Embodiment 126. The method of any one of embodiments 111-125, wherein the double-stranded nucleic acid comprises DNA.

Embodiment 127. The method of embodiment 126, wherein the DNA comprises DNA associated with histones.

Embodiment 128. The method of embodiment 127, wherein the DNA associated with histones is cell-free DNA.

Embodiment 129. The method of embodiment 127 or embodiment 128, wherein the cell-free DNA is not treated with a protease before combining with the zinc finger DNA-binding domain.

Embodiment 130. The method of any one of embodiments 111-129, further comprising adding an affinity binding partner on a solid support after fragmenting, wherein the tagged target fragments are bound to the solid support.

Embodiment 131. The method of embodiment 130, wherein the fragmenting is stopped before adding the affinity element on the solid support.

Embodiment 132. The method of embodiment 131, wherein the fragmenting is stopped by addition of a solution comprising proteinase K and/or SDS.

Embodiment 133. The method of any one of embodiments 111-132, wherein the combining a sample comprising a double-stranded nucleic acid with one or more transposome complex that is targeted comprises combining the sample with a zinc finger DNA-binding domain or a catalytically inactive endonuclease, wherein the zinc finger DNA-binding domain or catalytically inactive endonuclease is bound to a first binding partner, and adding the transposase and first and second transposons, wherein the transposase is bound to a second binding partner, wherein the transposase can bind to the zinc finger DNA-binding domain or catalytically inactive endonuclease by pairing of the first and second binding partners.

Embodiment 134. The method of embodiment 133, wherein the sample is combined with a zinc finger DNA-binding domain.

Embodiment 135. The method of embodiment 134, wherein the zinc finger DNA-binding domain is comprised in a zinc finger nuclease.

Embodiment 136. The method of embodiment 135, wherein the zinc finger nuclease is catalytically inactive.

Embodiment 137. The method of any one of embodiments 133-136, wherein the double-stranded nucleic acid comprises DNA.

Embodiment 138. The method of embodiment 137, wherein double-stranded nucleic acid comprises DNA associated with histones.

Embodiment 139. The method of embodiment 138, wherein the DNA associated with histones is cell-free DNA.

Embodiment 140. The method of embodiment 139, wherein the cell-free DNA is not treated with a protease before combining with the zinc finger DNA-binding domain.

Embodiment 141. The method of any one of embodiment 133-140, wherein the method comprises washing after the combining and before the adding.

Embodiment 142. The method of any one of embodiments 133-141, wherein the first transposome complex that is targeted and the second transposon complex that is targeted bind to opposite strands of the double-stranded nucleic acid, wherein the first transposome complex binds to a first transposome complex binding site and wherein the second transposome complex binds to a second transposome complex binding site.

Embodiment 143. The method of embodiment 142, wherein the first 5′ tagged target fragments and the second 5′ tagged target fragments comprise nucleic acid sequences comprised in in a region of the double-stranded nucleic acid between the first transposome complex binding site and the second transposome complex binding site.

Embodiment 144. The method of embodiment 143, wherein the first 5′ tagged target fragments and the second 5′ tagged fragments are at least partially complementary.

Embodiment 145. The method of any one of embodiments 133-144, wherein the transposome complexes are at an approximately equal stoichiometry to the target DNA.

Embodiment 146. The method of any one of embodiments 133-145, wherein divalent cations are absent during the combining.

Embodiment 147. The method of any one of embodiments 133-145, wherein Ca²⁺ and/or Mn²⁺ are present during the combining.

Embodiment 148. The method of any one of embodiments 133-145, further comprising adding one or more divalent cations to the sample after the combining and before the fragmenting.

Embodiment 149. The method of embodiment 148, wherein the divalent cation is Mg²⁺.

Embodiment 150. The method of any one of embodiments 133-149, further comprising treating the sample with an exonuclease after the combining and before the fragmenting.

Embodiment 151. The method of embodiment 150, comprising adding Mg²⁺ after the treating sample with an exonuclease and before the fragmenting.

Embodiment 152. The method of any one of embodiments 133-151, further comprising releasing the tagged fragments with proteinase K and/or SDS.

Embodiment 153. The method of any one of embodiments 111-152 or the kit or composition of embodiment 108-110, wherein the 5′ adaptor sequences comprised in the first transposome complex and the second transposome complex are different.

Embodiment 154. The method of any one of embodiments 111-153, wherein the catalytically inactive endonuclease or zinc finger DNA-binding domain comprised in the first transposome complex that is a targeted transposome complex and the second transposome complex that is a targeted transposome complex are different.

Embodiment 155. The method of embodiment 111-154, wherein the catalytically inactive endonuclease or zinc finger DNA-binding domain of the first transposome complex that is a targeted transposome complex and the second transposome complex that is a targeted transposome complex bind to different sequences of interest in a given region of interest in a target nucleic acid.

Embodiment 156. The method of any one of embodiments 111-155, wherein the fragmenting is performed at 45° C. to 65° C.

Embodiment 157. The method of embodiment 156, wherein the fragmenting is performed at 50° C. to 60° C.

Embodiment 158. The method of any one of embodiments 157, wherein the fragmenting is performed at 55° C.

Embodiment 159. The method of any one of embodiments 111-158, further comprising treating the plurality of 5′ tagged fragments with a polymerase and a ligase to extend and ligate the strands to produce fully double-stranded tagged fragments.

Embodiment 160. The method of any one of embodiments 111-159, further comprising sequencing one or more of the 5′ tagged fragments or fully double-stranded tagged fragments.

Embodiment 161. A method of characterizing desired samples in a mixed pool of samples comprising both desired samples and unwanted samples comprising to produce sequencing data from double-stranded nucleic acid, initially sequencing a library comprising a plurality of nucleic acid samples from the mixed pool, wherein each nucleic acid library comprises nucleic acids from a single sample and a unique sample barcode to distinguish the nucleic acids from the single sample from the nucleic acids from other samples in the library; analyzing the sequencing data and identifying unique sample barcodes associated with sequencing data from desired samples; performing a selection step on the library comprising enriching nucleic acid samples from desired samples and/or depleting nucleic acid samples from unwanted samples; and resequencing the nucleic acid library.

Embodiment 162. The method of embodiment 161, wherein the mixed pool of samples comprises a mixed pool of cells, a mixed pool of nuclei, or a mixed pool of high molecular weight DNA.

Embodiment 163. The method of embodiment 161 or embodiment 162, wherein the samples are cells, nuclei, or high molecular weight DNA.

Embodiment 164. The method of any one of embodiments 161-163, wherein the unique sample barcode is a unique cellular barcode.

Embodiment 165. The method of any one of embodiments 161-164, wherein the enriching step comprises hybrid capture, capture via catalytically inactive endonucleases, or unique sample barcode-specific amplification.

Embodiment 166. The method of embodiment 165, wherein the unique sample barcode-specific amplification is unique sample barcode-targeting PCR amplification.

Embodiment 167. The method of any one of embodiments 161-164, wherein the depletion step comprises hybrid capture, capture via catalytically inactive endonucleases, CRISPR digestion, or cleavage by a complex comprising a ShCAST (Scytonema hofmanni CRISPR associated transposase) coupled to guide RNA (gRNA).

Embodiment 168. The method of embodiment 167, wherein the hybrid capture comprises hybridizing a hybrid capture oligonucleotide to the unique sample barcode.

Embodiment 169. The method of embodiment 168, wherein the hybrid capture oligonucleotide is bound directly or indirectly to a solid support.

Embodiment 170. The method of embodiment 169, wherein the hybrid capture oligonucleotide is bound to a solid support through a biotin-streptavidin interaction.

Embodiment 171. The method of embodiment 167, wherein the CRISPR digestion is cleavage via a catalytically active endonuclease.

Embodiment 172. The method of embodiment 171, wherein the endonuclease is Cas9.

Embodiment 173. The method of embodiment 172, wherein the Cas9 is a Streptococcus canis Cas9.

Embodiment 174. The method of embodiment 173, wherein the Streptococcus canis Cas9 has minimal sequence constraint.

Embodiment 175. The method of any one of embodiments 171-174, wherein the endonuclease is a higher-fidelity mutant.

Embodiment 176. The method of embodiment 171, comprising cleavage by a complex comprising a ShCAST coupled to gRNA.

Embodiment 177. The transposome complex of any one of embodiments 171-176, wherein the endonuclease is comprised in a fusion protein together with FokI nuclease.

Embodiment 178. The method of any one of embodiments 171-177, wherein the endonuclease is associated with a guide RNA that binds to one or more unique sample barcode.

Embodiment 179. The method of embodiment 178, wherein guide RNAs are directed against unique sample barcodes associated with nucleic acids of unwanted samples.

Embodiment 180. The method of embodiment 178, wherein guide RNAs are directed against unique sample barcodes associated with nucleic acids of desired samples.

Embodiment 181. The transposome complex of any one of embodiments 178-180, wherein the guide RNA is a single guide.

Embodiment 182. The transposome complex of embodiment 181, wherein the single guide RNA comprises less than 20 nucleotides.

Embodiment 183. The transposome complex of embodiment 182, wherein the single guide RNA sequence comprises 15, 16, 17, 18, or 19 nucleotides.

Embodiment 184. The transposome complex of any one of embodiments 178-183, wherein the single guide RNA comprises a hairpin secondary structure.

Embodiment 185. The method of any one of embodiments 171-184, wherein the endonuclease is bound directly or indirectly to a solid support.

Embodiment 186. The method of embodiment 185, wherein the endonuclease is bound to a solid support through a biotin-streptavidin interaction.

Embodiment 187. The method of any one of embodiments 161-186, wherein the desired sample is a rare sample that is present in less than or equal to 1%, 0.1%, 0.01%, 0.001%, 0.0001%, 0.00001%, 0.000001%, 0.0000001%, 0.00000001%, or 0.0000000010% of a mixed pool of samples.

Embodiment 188. The method of embodiment 161-186, wherein the desired sample is a desired cell that is present in less than or equal to 1%, 0.1%, 0.01%, 0.001%, 0.00010%, 0.000010%, 0.0000010%, 0.00000010%, 0.000000010%, or 0.000000001%% of a mixed pool of cells.

Embodiment 189. The method of any one of embodiments 161-188, wherein the method comprises an amplification step before resequencing.

Embodiment 190. The method of embodiment 189, wherein the amplification step uses universal primers.

Embodiment 191. The method of any one of embodiments 161-190, wherein the nucleic acid libraries are prepared by tagmentation.

Embodiment 192. The method of any one of embodiments 161-191, wherein the method comprises a step of spatially separating the nucleic acid samples before incorporating a unique sample barcode.

Embodiment 193. The method of any one of embodiments 161-192, wherein the method comprises tagmentation prior to sequencing a plurality of nucleic acid samples from the mixed pool of samples.

Embodiment 194. The method of any one of embodiments 161-193, wherein a unique sample barcode is incorporated into each nucleic acid sample.

Embodiment 195. The method of any one of embodiments 161-194, wherein i5 and i7 sequences are incorporated into each nucleic acid sample.

Embodiment 196. The method of any one of embodiments 161-195, wherein universal primers are incorporated into each nucleic acid sample.

Embodiment 197. The method of any one of embodiments 196, wherein the universal primers are P5 and/or P7 primers.

Embodiment 198. The method of any one of embodiments 161-197, wherein the unique sample barcode is a single contiguous barcode.

Embodiment 199. The method of any one of embodiments 198, wherein the unique sample barcode is multiple discontiguous barcodes.

Embodiment 200. The method of embodiment 199, wherein the multiple discontiguous barcodes are separated by fixed sequences.

Embodiment 201. The method of any one of embodiments 161-200, wherein the amplification and resequencing steps are repeated once.

Embodiment 202. The method of any one of embodiments 161-200, wherein the amplification and resequencing steps are repeated more than once.

Embodiment 203. The method of any one of embodiments 161-202, wherein the nucleic acid is DNA.

Embodiment 204. The method of any one of embodiments 161-202, wherein the nucleic acid is RNA.

Embodiment 205. The method of embodiment 204, wherein the nucleic acid is rRNA.

Embodiment 206. The method of embodiment 205, wherein the nucleic acid is 16s rRNA.

Embodiment 207. The method of embodiment 205, wherein the nucleic acid is 18s rRNA.

Embodiment 208. The method of embodiment 203, wherein the nucleic acid is rDNA.

Embodiment 209. The method of any one of embodiments 161-208, wherein the nucleic acid is internal transcribed spacer nucleic acid.

Embodiment 210. The method of any one of embodiments 161-209, wherein the initial sequencing step does not comprise whole genome sequencing and the resequencing step comprises whole genome sequencing.

Embodiment 211. The method of any one of embodiments 161-209, wherein the initial sequencing step comprises targeted sequencing and the resequencing step comprises whole genome sequencing.

Embodiment 212. The method of embodiment 211, wherein the initial sequencing step comprises targeted sequencing with one or more gene-specific primers.

Embodiment 213. The method of embodiment 212, wherein the gene-specific primer comprises a universal primer tail.

Embodiment 214. The method of any one of embodiments 161-210, wherein the initial sequencing step comprises ribosomal sequencing and the resequencing step comprises whole genome sequencing.

Embodiment 215. The method of embodiment 214, wherein the ribosomal sequencing comprises 16s, 18s, or internal transcribed spacer sequencing.

Embodiment 216. The method of any one of embodiments 161-215, wherein the desired sample is a cell or nucleus.

Embodiment 217. The method of embodiment 216, wherein the desired sample is a cell.

Embodiment 218. The method of any one of embodiments 161-217, wherein the desired sample is a nucleus from a cell.

Embodiment 219. The method of any one of embodiments 161-217, wherein the desired sample is a human cell or a nucleus from a human cell.

Embodiment 220. The method of any one of embodiments 161-217, wherein the desired sample is a cancer cell or a nucleus from a cancer cell.

Embodiment 221. The method of any one of embodiments 161-220, wherein the desired cell or nucleus is or is from a specific desired cell type.

Embodiment 222. The method of any one of embodiments 161-221, wherein the desired sample has a mutation relative to other sample in the pool.

Embodiment 223. The method of any one of embodiments 161-222, wherein the desired sample is or is from a cancer cell or an immune cell.

Embodiment 224. The method of embodiment 223, wherein the desired sample is or is from a cancer stem cell.

Embodiment 225. The method of embodiment 223, wherein the desired sample is or is from a cancer cell in a liquid or tumor biopsy sample.

Embodiment 226. The method of embodiment 220, wherein the desired sample is or is from a cancer cell resistant to drug treatment.

Embodiment 227. The method of embodiment 220, wherein the desired sample is or is from a cancer cell that has at least one mutation relative to other cancer cells in the pool of cells.

Embodiment 228. The method of any one of embodiments 161-227, wherein the method is used for tracking cancer evolution.

Embodiment 229. The method of any one of embodiments 161-228, wherein the desired sample is or is from a cell having a somatic driver mutation.

Embodiment 230. The method of any one of embodiments 161-218, wherein the method is used for metagenomics.

Embodiment 231. The method of embodiment 230, wherein the method is used to sequence a microbe from an environmental sample.

Embodiment 232. The method of embodiment 231, wherein the method does not comprise culturing the microbe from the environmental sample.

Embodiment 233. The method of any one of embodiments 230-232, wherein the microbe comprises bacteria, fungi, archaea, fungi, algae, protozoa, or virus.

Embodiment 234. The method of any one of embodiments 161-233, wherein the desired sample has a single nucleotide variant (SNV).

Embodiment 235. The method of any one of embodiments 161-234, wherein the desired sample has a copy number variation (CNV).

Embodiment 236. The method of any one of embodiments 161-235, wherein the desired sample has a desired methylation pattern.

Embodiment 237. The method of any one of embodiments 161-236, wherein the desired sample has a desired expression pattern.

Embodiment 238. The method of any one of embodiments 161-237, wherein the desired sample has a desired epigenetic pattern.

Embodiment 239. The method of any one of embodiments 161-229 or 234-238, wherein the desired sample has a desired immune gene recombination.

Embodiment 240. The method of any one of embodiments 161-229 or 234-239, wherein the method includes TCR repertoire characterization.

Embodiment 241. The method of any one of embodiments 161-240, wherein the desired sample has a specific species type.

Embodiment 242. The method of any one of embodiments 230-238, wherein the desired sample is a pathogen.

Embodiment 243. The method of embodiment 242, wherein the desired sample is or is from a bacteria, fungi, archaea, fungi, algae, protozoa, or virus.

Embodiment 244. The method of any one of embodiments 161-243, wherein the method does not employ cell sorting-based enrichment methods.

Embodiment 245. The method of embodiment 244, wherein the method does not employ FACS.

Embodiment 246. The method of embodiment 245, wherein the method does not employ FACS based on cell size, morphology, or surface protein expression.

Embodiment 247. The method of any one of embodiments 161-246, wherein the method does not employ microfluidics.

Embodiment 248. The method of any one of embodiments 161-247, wherein the method does not employ whole genome amplification.

Embodiment 249. The method of embodiment 176, wherein:
a. the ShCAST comprises Cas12K;
b. the transposase comprises Tn5 or a Tn7-like transposase; and/or
c. at least one of the gRNA and the transposase is biotinylated, wherein at least one of the gRNA and transposase that is biotinylated is capable of coupling to a streptavidin-coated bead.

Embodiment 250. The method of embodiment 176 or 249, wherein depleting nucleic acid samples from unwanted samples is performed in a fluid having a condition for limiting binding of the transposase comprised in the complex to double-stranded nucleic acid.

Embodiment 251. The method of embodiment 250, wherein the condition for limiting binding of the transposase comprised in the complex to double-stranded nucleic acid is a magnesium concentration of 15 mM or lower.

Embodiment 252. The method of embodiment 250 or 251, wherein the condition for limiting binding of the transposase comprised in the complex to double-stranded nucleic acid is a concentration of transposase of 50 nM or lower.

Embodiment 253. The method of embodiment 176 or 249, wherein depleting nucleic acid samples from unwanted samples comprises:
a. binding complexes to a double-stranded nucleic acid under conditions that inhibit binding of the nucleic acid by the transposase comprised in the complex; and
b. after the binding, promoting cleavage of the nucleic acid by the complex.

Embodiment 254. The method of embodiment 253, wherein (1) a transposase is absent during the binding and (2) promoting cleavage comprises adding a transposase.

Embodiment 255. The method of embodiment 253, wherein (1) a transposase is at low levels during the binding and (2) promoting cleavage comprises adding a transposase.

Embodiment 256. The method of any one of embodiments 252-255, wherein (1) a transposase is reversibly deactivated during the binding and (2) promoting cleavage comprises activating the transposase.

Embodiment 257. The method of embodiment 256, wherein (1) the transposase is reversibly deactivated due to lack of one or more transposon and (2) activating the transposase comprises providing one or more transposons.

Embodiment 258. A composition comprising (1) a target nucleic acid comprising one or more nucleic acid sequences of interest and (2) a plurality of targeted transposome complexes according to embodiment 59 each comprising an ShCAST coupled to gRNA, wherein the ShCAST has an amplification adapter coupled thereto, and wherein each of the targeted transposome complexes is hybridized to a nucleic acid sequence of interest.

Embodiment 259. The composition of embodiment 258, wherein the ShCAST comprises Cas12K, further comprising a fluid having a condition promoting hybridization of the Cas12K comprised in the complexes to the one or more nucleic acid sequences of interest and inhibiting binding of the transposases comprised in the complexes.

Embodiment 260. The composition of embodiment 259, wherein the condition of the fluid further comprises the absence of a sufficient amount of magnesium ions for activity of the transposases, optionally wherein the magnesium concentration is 15 mM or lower.

Embodiment 261. The composition of embodiment 258, comprising a fluid having a condition promoting activity of the transposases, and in which the transposases are capable of adding the amplification adapters to locations in the target nucleic acid.

Embodiment 262. The composition of embodiment 261, wherein the condition of the fluid comprises the presence of a sufficient amount of magnesium ions for activity of the transposases, optionally wherein the magnesium concentration is 15 mM or higher.

Embodiment 263. The composition of any one of embodiments 258-262, wherein the ShCAST comprises Cas12K.

Embodiment 264. The composition of any one of embodiments 258-263, wherein the transposase comprises Tn5 or a Tn7-like transposase.

Embodiment 265. The composition of any one of embodiments 258-264, wherein the adapter comprises at least one of a P5 adapter and a P7 adapter.

Embodiment 266. The composition of any one of embodiments 258-265, wherein the target nucleic acid comprises double-stranded DNA.

Embodiment 267. The composition of any one of embodiments 258-266, wherein at least one of the gRNA and the transposase is biotinylated, the composition further comprising a streptavidin-coated bead to which the at least one of the gRNA and transposase that is biotinylated is coupled.

Embodiment 268. The method of any one of embodiments 111-113, wherein the first and/or second targeted transposome complex comprise the targeted transposome complex of embodiment 59.

Embodiment 269. The method of embodiment 268, wherein the method is performed in a fluid having a condition for limiting binding of the transposase comprised in the complex.

Embodiment 270. The method of embodiment 269, wherein the condition for limiting binding of the transposase comprised in the complex is a magnesium concentration of 15 mM or lower.

Embodiment 271. The method of embodiment 269 or 270, wherein the condition for limiting binding of the transposase comprised in the complex is a concentration of transposase of 50 nM or lower.

Embodiments 272. The method of embodiment 268, wherein the method comprises:
a. binding the complex to a double-stranded nucleic acid under conditions that inhibit binding of the double-stranded nucleic acid by the transposase comprised in the complex; and
b. after the binding, promoting cleavage of the double-stranded nucleic acid by the complex.

Embodiment 273. The method of embodiment 272, wherein the (1) a transposase is absent during the binding and (2) promoting cleavage comprises adding a transposase.

Embodiment 274. The method of any one of embodiments 271-273, wherein (1) a transposase is at low levels during the binding and (2) promoting cleavage comprises adding a transposase.

Embodiment 275. The method of any one of embodiments 271-274, wherein (1) a transposase is reversibly deactivated during the binding and (2) promoting cleavage comprises activating the transposase.

Embodiment 276. The method of embodiment 275, wherein (1) the transposase is reversibly deactivated due to lack of one or more transposon and (2) activating the transposase comprises providing one or more transposons.

Embodiment 277, The method of any one of embodiments 268-276, wherein the transposases add the amplification adapters to locations in the double-stranded nucleic acid.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides exemplary populations of samples that may be used with the present method. In metagenomics samples, rare samples of interest might be bacteria that express a certain plasmid (shaded inset) or the presence of a rare virus (black inset) within the sample. In oncology samples, a rare sample of interest may be cells that express a somatic driver mutation (insets). Generally, data from these rare samples might be difficult to evaluate, since data from abundant samples would overwhelm sequencing results.

FIG. 2 shows a representative method for metagenomics uses. A single cell library (sc-library) is generated, comprising a plurality of libraries from single cells. Using the present methods fragments in each library from a single cell are uniquely tagged, such as with a unique cell barcode (UBC). After an initial sequencing to identify UBCs associated with the desired samples (such as those from rare cells of interest), selection and resequencing of desired samples is performed. This method avoids data from cells of interest being lost or overwhelmed by the large amount of sequencing data generated from abundant samples. In the absence of the present quality control methods, rare samples of interest may be lost from bioinformatic analysis.

FIG. 3 shows a representative method of sequencing-based sorting and selection of libraries from rare single cells. After a library is constructed, an initial sequencing can be performed (such as 16s sequencing) to determine desired samples. These desired samples may be libraries generated from rare cells within the total population of single cells. Selection of desired samples is then performed, by either enrichment or depletion, based on UBCs associated with the library fragments from single cells of interest. Selection can be performed via a number of different means, such as by using unique sample barcode-specific PCR, hybrid capture, or capture by a catalytically inactive Cas9. After selection of desired samples, comprehensive sequencing can be performed to better understand the characteristics of the rare cells of interest.

FIG. 4 shows methods of selection for use with a library generated from a mixed population via a Sci-RNA3 method. Similar methods could be used with libraries generated by other means.

FIG. 5 shows a method of generating a library using a modified SCI-seq method to yield contiguous barcodes.

FIG. 6 shows a method for generating a library using a synthetic linked DNA library constructed with physically addressable barcodes.

FIG. 7 shows a method of performing initial targeted sequencing.

FIG. 8 shows a variety of means to increase the specificity of an endonuclease (such as Cas9) that may be used for selection.

FIG. 9 provides an overview of recombinase-mediated targeted transposition. Recombinase (Rec)-coated targeting oligonucleotides (oligos) can bind to a genomic DNA to be targeted. The recombinase mediates strand invasion to localize transposomes to regions of interest. Subsequent transposition can insert P5/P7 sequences into the genomic DNA, after which fragments of the region of interest can be generated.

FIG. 10 shows an overview of targeted transposition based on targeted oligonucleotides. Single-stranded genomic target DNA can be denatured, after which targeted oligonucleotides can hybridize (hyb) one or more nucleic acid sequence of interest within the single-stranded DNA (ssDNA). Transposases and transposons can then be added. As transposases bind to regions of double-stranded nucleic acid, transposition is targeted to regions where the targeted oligonucleotides have bound. In contrast, transposases would not bind to other regions of the ssDNA. Transposition can insert P5/P7 sequences into the genomic DNA, after which fragments of the region of interest can be generated.

FIG. 11 shows a method of generating a library using a targeted transposome complex comprising a fusion protein of a catalytically inactive endonuclease (deactivated or dCas9 in this embodiment) linked to a transposase (Tn5 in this embodiment). The single guide RNA (sgRNA) associated with the dCas9 targets the fusion protein to bind specific nucleotide sequences within the target nucleic acid. This binding can be done under conditions wherein dCas9 binding is active, but the transposase is inactive (for example, in the presence of Ca²⁺ and/or Mn²⁺). After binding of the fusion protein, tagmentation via the transposase can be activated with Mg²⁺ to allow generation of tagged library fragments using a protocol similar to that for Nextera preparations. The resulting fragments can then be sequenced.

FIGS. 12A-12D presents a variety of means to produce a targeted transposome complex comprising a catalytically inactive endonuclease and a transposase. The targeted transposome complex may comprise a fusion protein, wherein the endonuclease and the transposase are expressed as one protein (A). This fusion protein may comprise a linker between the endonuclease and the transposase. Alternatively, a binding pair (such as streptavidin and biotin) may be used to associate the transposase and endonuclease (B). In any embodiment described herein, the guide RNA may be truncated (e.g., comprise less than 20 nucleotides), such as comprising 17 nucleotides, as truncated guide RNAs can increase the specificity for one or more sequence of interest in a target nucleic acid. A single guide RNA (sgRNA) may associate with a transposon, such as a sgRNA associating with a transposon comprising a transposon end sequence and Tn5 adaptors, such as A14 and B15 (C). The association of the sgRNA and transposon may be mediated by a region of complementary sequence. Further, a contiguous sgRNA-transferred strand oligonucleotide (single oligonucleotide) may be used (D).

FIG. 13 shows a variety of embodiments that can increase specificity of targeted transposome complexes comprising a catalytically inactive endonuclease. Truncated guide RNAs can increase specificity for specific sequences of interest in a target nucleic acid, and endonucleases with minimal sequence constraint for a specific protospacer adjacent motif (PAM) can allow greater target design space. Hairpin secondary structures, such as toehold-blocked guide RNA, can also be used to increase specificity.

FIGS. 14A-14C show how a targeted transposome complex comprising a fusion protein of a dCas9 and a transposase can be used to mediate fragmentation of an enrichment target region. The fusion protein would scan the target nucleic acid (such as DNA) looking for a sequence of interest that binds to the guide RNA of the dCas9 in close proximity to a PAM (A). Once it finds the sequence of interest, high-specificity binding of the dCas9 can be achieved with tagmentation (such as initially contacting without divalent ions or with Ca²⁺ or Mn²⁺ to allow binding and conformation change of an sgRNA-Cas9 without allowing tagmentation by a transposase). After allowing for binding of the dCas9, tagmentation via the transposase (such as Tn5) is initiated by adding Mg²⁺. Exonuclease treatment before adding Mg²⁺ may allow extra specificity by removing non-Cas9 protected regions of the target DNA. After cleavage, the DNA fragments can be released by Proteinase K and/or SDS. These methods can lead to a high percentage of fragments in a library comprising the enrichment target region. After release of the DNA, extension and gap-fill ligation can be performed (C).

FIG. 15 shows use of zinc finger nuclease (ZNF)-associated transposomes for generating a targeted library from cell-free DNA (cfDNA) in plasma. The zinc finger DNA-binding domain or ZNF can target the transposome complex to sites within cfDNA, even when the cfDNA is associated with histones.

FIGS. 16A and 16B schematically illustrate example compositions (A) and operations in a process flow (B) for ShCAST (Scytonema hofmanni CRISPR associated transposase) targeted library preparation and enrichment.

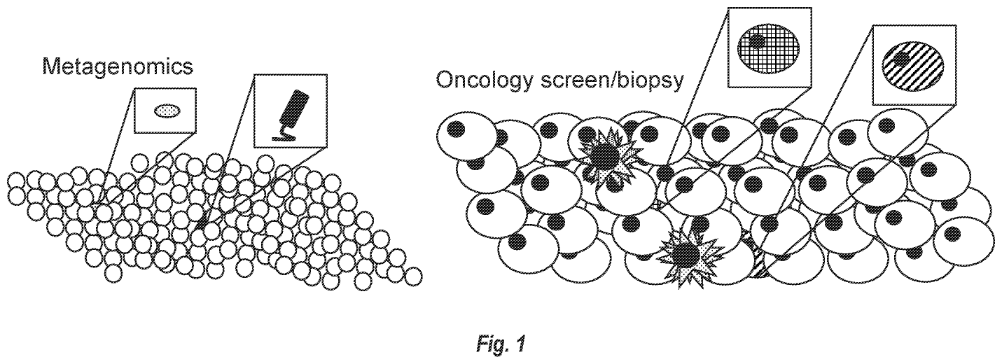

Table 2 below provides descriptions for the labeled components.

TABLE 2
Description of labels

| Label | Description |
|---|---|
| 6000 | ShCAST comprising Cas12k and a transposase |
| 6001 | Cas12k |
| 6002 | Transposase (e.g., Tn7-like transposase or Tn5) |
| 6003 | DNA for inserting (e.g. transposon comprising one or more |
| | adapter sequence to enable tagmentation) |
| 6004 | guide RNA (gRNA) |
| 6005 | Tag (e.g., biotin) |
| 6010 | Process flow using ShCAST comprising Cas12k and a |
| | transposase |
| 6011 | Target nucleic acid (e.g., genomic DNA) |
| 6012 | Solid support for binding tag (e.g., streptavidin beads) |

### DESCRIPTION OF THE SEQUENCES

Table 1 provides a listing of certain sequences referenced herein.

TABLE 1
Description of the Sequences

| | | SEQ |
|---|---|---|
| | | ID |
| Description | Sequences | NO |
| 40-mer_A14_ME | GCCTTTTGTA | 1 |
| | ATAATTACCG | |
| | CAGCTCGCAG | |
| | GCCAATTTCG | |
| | TCGTCGGCAG | |
| | CGTCAGATGT | |
| | GTATAAGAGA | |
| | CAG | |
| ME′ (3′ to 5′) | TCTACACATA | 2 |
| | TTCTCTGTC | |
| 40-mer_B15_ME | TCAACTTTAC | 3 |
| | CATTATTCTG | |
| | CTGGTTAGAC | |
| | TGGTCGTTCC | |
| | TTCGGTTCTA | |
| | GTCTCGTGGG | |
| | CTCGGAGATG | |
| | TGTATAAGA | |
| | GACAG | |
| ME′ (5′ to 3′) | CTGTCTCTTA | 4 |
| | TACACATCT | |
| Single- | GCCTTTTGTA | 5 |
| stranded 30- | ATAATTACCG | |
| mer DNA | CAGCTCGCAG | |
| primer | | |
| targeting | | |
| PhiX DNA | | |
| Single- | GGCAGAAAGA | 6 |
| stranded 30- | GGTAACGCAG | |
| mer DNA | CACCGGAACG | |
| primer | | |
| targeting | | |
| PhiX DNA | | |
| P5 | AATGATACGG | 7 |
| | CGACCACCGA | |
| | GAUCTACAC | |
| P7 | CAAGCAGAAG | 8 |
| | ACGGCATACG | |
| | AG*AT | |

### DESCRIPTION OF THE EMBODIMENTS

Described herein are a variety of targeted transposome complexes. As used herein, a “targeted transposome complex” refers to a transposome complex that is targeted to one or more nucleic acid sequences of interest in a target nucleic acid.

### I. Targeted Transposome Complexes

This application describes a number of different targeted transposome complexes, wherein the transposomes are targeted to nucleic acid sequences of interest in a target nucleic acid. In some embodiments, a targeted transposome complexes comprises a component that can bind to one or more nucleic acid sequences of interest in a target nucleic acid. Based on this binding, a targeted transposome complexes can mediate transposition at a region of interest in a target nucleic acid.

A targeted transposome complex can be any transposome complex that has non-random binding to a target nucleic acid. Thus, a targeted transposome complex may differ from a non-targeted transposome complex that randomly binds to sequences in the target nucleic acid. For example, a targeted transposome complex may comprise a component that binds to one or more nucleic acid sequences of interest in a target nucleic acid. Methods using these targeted transposome complexes can be used to generate targeted libraries, wherein fragments comprise regions of interest in a target nucleic acid.

A number of different types of targeted transposome complexes are described herein.

A. Transposome Complexes

Generally, the present transposon complexes comprise a transposase and a first and second transposon, along with one or more component that mediates targeting to one or more nucleic acid sequence of interest.

A “transposome complex,” as used herein, is comprised of at least one transposase (or other enzyme as described herein) and a transposon recognition sequence. In some such systems, the transposase binds to a transposon recognition sequence to form a functional complex that is capable of catalyzing a transposition reaction. In some aspects, the transposon recognition sequence is a double-stranded transposon end sequence. The transposase binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting in a cleavage event. Exemplary transposition procedures and systems that can be readily adapted for use with the transposases.

A “transposase” means an enzyme that is capable of forming a functional complex with a transposon end-containing composition (e.g., transposons, transposon ends, transposon end compositions) and catalyzing insertion or transposition of the transposon end-containing composition into a double-stranded target nucleic acid. A transposase as presented herein can also include integrases from retrotransposons and retroviruses.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. More examples include IS5, Tn10, Tn903, IS911, and engineered versions of transposase family enzymes. The methods described herein could also include combinations of transposases, and not just a single transposase.

In some embodiments, the transposase is a Tn5, Tn7, MuA, or Vibrio harveyi transposase, or an active mutant thereof. In other embodiments, the transposase is a Tn5 transposase or a mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, which is incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998). In one embodiment, a transposase recognition site that forms a complex with a hyperactive Tn5 transposase is used (e.g., EZ-Tn5TM Transposase, Epicentre Biotechnologies, Madison, Wis.). In some embodiments, the Tn5 transposase is a wild-type Tn5 transposase.

As used throughout, the term transposase refers to an enzyme that is capable of forming a functional complex with a transposon-containing composition (e.g., transposons, transposon compositions) and catalyzing insertion or transposition of the transposon-containing composition into the double-stranded target nucleic acid with which it is incubated in an in vitro transposition reaction. A transposase of the provided methods also includes integrases from retrotransposons and retroviruses. Exemplary transposases that can be used in the provided methods include wild-type or mutant forms of Tn5 transposase and MuA transposase.

A “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The method of this disclosure is exemplified by employing a transposition complex formed by a hyperactive Tn5 transposase and a Tn5-type transposon end or by a MuA or HYPERMu transposase and a Mu transposon end comprising R1 and R2 end sequences (See e.g., Goryshin, I. and Reznikoff, W. S., J. Biol. Chem., 273: 7367, 1998; and Mizuuchi, Cell, 35: 785, 1983; Savilahti, H, et al., EMBO J., 14: 4893, 1995; which are incorporated by reference herein in their entireties). However, any transposition system that is capable of inserting a transposon end in a random or in an almost random manner with sufficient efficiency to tag target nucleic acids for its intended purpose can be used in the provided methods. Other examples of known transposition systems that could be used in the provided methods include but are not limited to Staphylococcus aureus Tn552, Ty1, Transposon Tn7, Tn/O and IS 10, Mariner transposase, Tel, P Element, Tn3, bacterial insertion sequences, retroviruses, and retrotransposon of yeast (See, e.g., Colegio O R et al, J. Bacteriol., 183: 2384-8, 2001; Kirby C et al, Mol. Microbiol., 43: 173-86, 2002; Devine S E, and Boeke J D., Nucleic Acids Res., 22: 3765-72, 1994; International Patent Application No. WO 95/23875; Craig, N L, Science. 271: 1512, 1996; Craig, N L, Review in: Curr Top Microbiol Immunol., 204: 27-48, 1996; Kleckner N, et al., Curr Top Microbiol Immunol., 204: 49-82, 1996; Lampe D J, et al., EMBO J., 15: 5470-9, 1996; Plasterk R H, Curr Top Microbiol Immunol, 204: 125-43, 1996; Gloor, G B, Methods Mol. Biol, 260: 97-1 14, 2004; Ichikawa H, and Ohtsubo E., J Biol. Chem. 265: 18829-32, 1990; Ohtsubo, F and Sekine, Y, Curr. Top. Microbiol. Immunol. 204: 1-26, 1996; Brown P O, et al, Proc Natl Acad Sci USA, 86: 2525-9, 1989; Boeke J D and Corces V G, Annu Rev Microbiol. 43: 403-34, 1989; which are incorporated herein by reference in their entireties).

The method for inserting a transposon into a target sequence can be carried out in vitro using any suitable transposon system for which a suitable in vitro transposition system is available or can be developed based on knowledge in the art. In general, a suitable in vitro transposition system for use in the methods of the present disclosure requires, at a minimum, a transposase enzyme of sufficient purity, sufficient concentration, and sufficient in vitro transposition activity and a transposon with which the transposase forms a functional complex with the respective transposase that is capable of catalyzing the transposition reaction. Suitable transposase transposon end sequences that can be used include but are not limited to wild-type, derivative or mutant transposon end sequences that form a complex with a transposase chosen from among a wild-type, derivative or mutant form of the transposase.

In some embodiments, the transposase comprises a Tn5 transposase. In some embodiments, the Tn5 transposase is hyperactive Tn5 transposase.

In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adaptor sequence in each monomer and the second population has a different adaptor sequence in each monomer.

The term “transposon end” refers to a double-stranded nucleic acid DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

The term “transferred strand” refers to the transferred portion of both transposon ends. Similarly, the term “non-transferred strand” refers to the non-transferred portion of both “transposon ends.” The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure. Additional examples of transposome structure and methods of preparing and using transposomes can be found in the disclosure of US 2010/0120098, the content of which is incorporated herein by reference in its entirety.

In some embodiments, the transposome complexes comprise a first transposon comprising a 3′ transposon end sequence and a 5′ adaptor sequence. In some embodiments, the transposome complexes comprise a second transposon comprising a 5′ transposon end sequence, wherein the 5′ transposon end sequence is complementary to the 3′ transposon end sequence.

Thus, in some embodiments, the transposon composition comprises a transferred strand with one or more other nucleotide sequences 5′ of the transferred transposon sequence, e.g., an adaptor sequence. In some embodiments, the adapter sequence is a tag sequence. In addition to the transferred transposon sequence, the tag can have one or more other tag portions or tag domains.

“Tagmentation,” as used herein, refers to the use of transposase to fragment and tag nucleic acids. Tagmentation includes the modification of DNA by a transposome complex comprising transposase enzyme complexed with one or more tag (such as adaptor sequences) comprising transposon end sequences (referred to herein as transposons). Tagmentation thus can result in the simultaneous fragmentation of the DNA and ligation of the adaptors to the 5′ ends of both strands of duplex fragments.

While a number of targeted transposome complexes are described in this application, it is understood that some methods may employ both targeted transposome complexes and non-targeted transposome complexes.

B. Immobilized Transposome Complexes

In some embodiments, a transposome complex is immobilized to a solid support.

In some embodiments, the transposome complexes are present on the solid support at a density of at least 10³, 10⁴, 10⁵, or 10⁶ complexes per mm².

In some embodiments, the lengths of the double-stranded fragments in the immobilized library are adjusted by increasing or decreasing the density of transposome complexes on the solid support.

A number of different types of immobilized transposomes can be used in these methods, as described in U.S. Pat. No. 9,683,230, which is incorporated herein in its entirety.

In the methods and compositions presented herein, transposome complexes are immobilized to the solid support. In some embodiments, the transposome complexes and/or capture oligonucleotides are immobilized to the support via one or more polynucleotides, such as a polynucleotide comprising a transposon end sequence. In some embodiments, the transposome complex may be immobilized via a linker molecule coupling the transposase enzyme to the solid support. In some embodiments, both the transposase enzyme and the polynucleotide are immobilized to the solid support. When referring to immobilization of molecules (e.g. nucleic acids) to a solid support, the terms “immobilized” and “attached” are used interchangeably herein and both terms are intended to encompass direct or indirect, covalent or non-covalent attachment, unless indicated otherwise, either explicitly or by context. In some embodiments, covalent attachment may be used, but generally all that is required is that the molecules (e.g. nucleic acids) remain immobilized or attached to the support under the conditions in which it is intended to use the support, for example in applications requiring nucleic acid amplification and/or sequencing.

Certain embodiments may make use of solid supports comprised of an inert substrate or matrix (e.g. glass slides, polymer beads etc.) which has been functionalized, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass, particularly polyacrylamide hydrogels as described in WO 2005/065814 and US 2008/0280773, the contents of which are incorporated herein in their entirety by reference. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

The terms “solid surface,” “solid support” and other grammatical equivalents herein refer to any material that is appropriate for or can be modified to be appropriate for the attachment of the transposome complexes. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. Particularly useful solid supports and solid surfaces for some embodiments are located within a flow cell apparatus. Exemplary flow cells are set forth in further detail below.

In some embodiments, the solid support comprises a patterned surface suitable for immobilization of transposome complexes in an ordered pattern. A “patterned surface” refers to an arrangement of different regions in or on an exposed layer of a solid support. For example, one or more of the regions can be features where one or more transposome complexes are present. The features can be separated by interstitial regions where transposome complexes are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. In some embodiments, the transposome complexes are randomly distributed upon the solid support. In some embodiments, the transposome complexes are distributed on a patterned surface. Exemplary patterned surfaces that can be used in the methods and compositions set forth herein are described in U.S. application Ser. No. 13/661,524 or US Pat. App. Publ. No. 2012/0316086 A1, each of which is incorporated herein by reference.

In some embodiments, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The composition and geometry of the solid support can vary with its use. In some embodiments, the solid support is a planar structure such as a slide, chip, microchip and/or array. As such, the surface of a substrate can be in the form of a planar layer. In some embodiments, the solid support comprises one or more surfaces of a flow cell. The term “flow cell” as used herein refers to a chamber comprising a solid surface across which one or more fluid reagents can be flowed. Examples of flow cells and related fluidic systems and detection platforms that can be readily used in the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support or its surface is non-planar, such as the inner or outer surface of a tube or vessel. In some embodiments, the solid support comprises microspheres or beads. By “microspheres” or “beads” or “particles” or grammatical equivalents herein is meant small discrete particles. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and teflon, as well as any other materials outlined herein for solid supports may all be used. “Microsphere Selection Guide” from Bangs Laboratories, Fishers Ind. is a helpful guide. In certain embodiments, the microspheres are magnetic microspheres or beads.

The beads need not be spherical; irregular particles may be used. Alternatively or additionally, the beads may be porous. The bead sizes range from nanometers, i.e. 100 nm, to millimeters, i.e. 1 mm, with beads from 0.2 micron to 200 microns, or from 0.5 to 5 microns, although in some embodiments smaller or larger beads may be used.

The density of these surface bound transposomes can be modulated by varying the density of the first polynucleotide or by the amount of transposase added to the solid support. For example, in some embodiments, the transposome complexes are present on the solid support at a density of at least 103, 104, 105, or 106 complexes per mm2.

Attachment of a nucleic acid to a support, whether rigid or semi-rigid, can occur via covalent or non-covalent linkage(s). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference. In some embodiments, a nucleic acid or other reaction component can be attached to a gel or other semisolid support that is in turn attached or adhered to a solid-phase support. In such embodiments, the nucleic acid or other reaction component will be understood to be solid-phase.

In some embodiments, the solid support comprises microparticles, beads, a planar support, a patterned surface, or wells. In some embodiments, the planar support is an inner or outer surface of a tube.

In some embodiments, a solid support has a library of tagged DNA fragments immobilized thereon prepared.

In some embodiments, solid support comprises capture oligonucleotides and a first polynucleotide immobilized thereon, wherein the first polynucleotide comprises a 3′ portion comprising a transposon end sequence and a first tag.

In some embodiments, the solid support further comprises a transposase bound to the first polynucleotide to form a transposome complex.

In some embodiments, a solid support comprises capture oligonucleotides and a second polynucleotide immobilized thereon, wherein the second polynucleotide comprises a 3′ portion comprising a transposon end sequence and a second tag.

In some embodiments, the solid support further comprises a transposase bound to the second polynucleotide to form a transposome complex.

In some embodiments, a kit comprises a solid support as described herein. In some embodiments, a kit further comprises a transposase. In some embodiments, a kit further comprises a reverse transcriptase polymerase. In some embodiments, a kit further comprises a second solid support for immobilizing DNA.

A wide variety of different means of immobilizing transposome complexes have been described, such as those described in WO 2018/156519, which is incorporated herein in its entirety. In some embodiments, the first transposon comprised in the targeted transposome complex comprises an affinity element. In some embodiments, the affinity element is attached to the 5′ end of the first transposon. In some embodiments, the first transposon comprises a linker. In some embodiments, the linker has a first end attached to the 5′ end of the first transposon and a second end attached to an affinity element.

In some embodiments, the targeted transposome complex further comprises a second transposon complementary to at least a portion of the first transposon end sequence. In some embodiments, the second transposon comprises an affinity element. In some embodiments, the affinity element is attached to the 3′ end of the second transposon. In some embodiments, the second transposon comprises a linker. In some embodiments, the linker has a first end attached to the 3′ end of the second transposon and a second end attached to an affinity element.

In some embodiments, the affinity element is biotin.

C. Solution-Phase Transposome Complexes

Targeted transposome complexes may be solution-phase transposome complexes. These solution-phase transposome complexes may be mobile and not immobilized to a solid support. In some embodiments, solution-phase targeted transposome complexes are used to generate tagged fragments in solution.

Further, present methods may comprise steps involving solution-phase transposome complexes. For example, a method presented herein can further comprise a step of providing transposome complexes in solution and contacting the solution-phase transposome complexes with the immobilized fragments under conditions whereby the DNA is fragmented by the transposome complexes solution; thereby obtaining immobilized nucleic acid fragments having one end in solution. In some embodiments, the transposome complexes in solution can comprise a second tag, such that the method generates immobilized nucleic acid fragments having a second tag, the second tag in solution. The first and second tags can be different or the same.

In some embodiments, the method further comprises contacting solution-phase transposome complexes with immobilized DNA fragments under conditions whereby the DNA fragments are further fragmented by the solution-phase transposome complexes; thereby obtaining immobilized nucleic acid fragments having one end in solution.

In some embodiments, the solution-phase transposome complexes comprise a second tag, thereby generating immobilized nucleic acid fragments having a second tag in solution. In some embodiments, the first and second tags are different. In some embodiments, at least 50%, 55%, 60%, 65%, 70%, 75%, 80%, 85%, 90%, 95%, 96%, 97%, 98%, or 99% of the solution-phase transposome complexes comprise a second tag.

In some embodiments, one form of surface bound transposome is predominantly present on the solid support. For example, in some embodiments, at least 50%, 55%, 60%, 65%, 70%, 75%, 80%, 85%, 90%, 95%, 96%, 97%, 98%, or 99% of the tags present on said solid support comprise the same tag domain. In such embodiments, after an initial tagmentation reaction with surface bound transposomes, at least 50%, 55%, 60%, 65%, 70%, 75%, 80%, 85%, 90%, 95%, 96%, 97%, 98%, or 99% of the bridge structures comprise the same tag domain at each end of the bridge. A second tagmentation reaction can be performed by adding transposomes from solution that further fragment the bridges. In some embodiments, most or all of the solution phase transposomes comprise a tag domain that differs from the tag domain present on the bridge structures generated in a first tagmentation reaction. For example, in some embodiments, at least 50%, 55%, 60%, 65%, 70%, 75%, 80%, 85%, 90%, 95%, 96%, 97%, 98%, or 99% of the tags present in the solution phase transposomes comprise a tag domain that differs from the tag domain present on the bridge structures generated in the first tagmentation reaction.

In some embodiments, the length of the templates is longer than what can be suitably amplified using standard cluster chemistry. For example, in some embodiments, the length of templates is at least 100 bp, 200 bp, 300 bp, 400 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2100 bp, 2200 bp, 2300 bp, 2400 bp, 2500 bp, 2600 bp, 2700 bp, 2800 bp, 2900 bp, 3000 bp, 3100 bp, 3200 bp, 3300 bp, 3400 bp, 3500 bp, 3600 bp, 3700 bp, 3800 bp, 3900 bp, 4000 bp, 4100 bp, 4200 bp, 4300 bp, 4400 bp, 4500 bp, 4600 bp, 4700 bp, 4800 bp, 4900 bp, 5000 bp, 10000 bp, 30000 bp or 100,000 bp. In such embodiments, then a second tagmentation reaction can be performed by adding transposomes from solution that further fragment the bridges, as described in U.S. Pat. No. 9,683,230, which is incorporated herein in its entirety. The second tagmentation reaction can thus remove the internal span of the bridges, leaving short stumps anchored to the surface that can converted into clusters ready for further sequencing steps. In particular embodiments, the length of the template can be within a range defined by an upper and lower limit selected from those exemplified above.

D. Adaptors and Tags

In some embodiments, a first transposon comprises a 3′ transposon end sequence and a 5′ adaptor sequence. In some embodiments, the 5′ adaptor sequence is a tag sequence. Fragmentation mediated by transposome complexes comprising a first transposon comprising a 3′ transposon end sequence and a 5′ tag can be used in methods to generate a library of tagged fragments.

In some embodiments, the adaptor sequence comprises a primer sequence, an index tag sequence, a capture sequence, a barcode sequence, a cleavage sequence, or a sequencing-related sequence, or a combination thereof. As used herein, a sequencing-related sequence may be any sequence related to a later sequencing step. A sequencing-related sequence may work to simplify downstream sequencing steps. For example, a sequencing-related sequence may be a sequence that would otherwise be incorporated via a step of ligating an adaptor to nucleic acid fragments. In some embodiments, the adaptor sequence comprises a P5 or P7 sequence (or their complement) to facilitate binding to a flow cell in certain sequencing methods.

The terms “tag” as used herein refers to a portion or domain of a polynucleotide that exhibits a sequence for a desired intended purpose or application. Tag domains can comprise any sequence provided for any desired purpose. For example, in some embodiments, a tag domain comprises one or more restriction endonuclease recognition sites. In some embodiments, a tag domain comprises one or more regions suitable for hybridization with a primer for a cluster amplification reaction. In some embodiments, a tag domain comprises one or more regions suitable for hybridization with a primer for a sequencing reaction. It will be appreciated that any other suitable feature can be incorporated into a tag domain. In some embodiments, the tag domain comprises a sequence having a length from 5 bp to 200 bp. In some embodiments, the tag domain comprises a sequence having a length from 10 bp to 100 bp. In some embodiments, the tag domain comprises a sequence having a length from 20 bp to 50 bp. In some embodiments, the tag domain comprises a sequence having a length of 5, 6, 7, 8, 9, 10, 20, 30, 40, 50, 60, 70, 80, 90, 100, 150 or 200 bp.

The tag can include one or more functional sequences or components (e.g., primer sequences, anchor sequences, universal sequences, spacer regions, or index tag sequences) as needed or desired.

In some embodiments, the tag comprises a region for cluster amplification. In some embodiments, the tag comprises a region for priming a sequencing reaction.

In some embodiments, the method further comprises amplifying the fragments on the solid support by reacting a polymerase and an amplification primer corresponding to a portion of the first transposon. In some embodiments, a portion of the first transposon comprises an amplification primer. In some embodiments, the tag of the first transposon comprises an amplification primer.

In some embodiments a tag comprises an A14 primer sequence. In some embodiments, a tag comprises a B15 primer sequence.

In some embodiments, transposomes on an individual bead carry a unique index, and if a multitude of such indexed beads are employed, phased transcripts will result.

E. Targeted Transposome Complexes Comprising a Targeting Oligonucleotide Coated with a Recombinase

In some embodiments, a targeted transposome complex comprises a targeting oligonucleotide. As used herein, a “targeting oligonucleotide” is an oligonucleotide that can bind to one or more nucleic acid sequences of interest. In some embodiments, the targeting oligonucleotide is coated with a recombinase. The targeting oligonucleotide may be used to direct binding of the transposome complex to one or more nucleic acid sequences of interest within the target nucleic acid.

In some embodiments, a targeted transposome complex comprises a transposase, a first transposon comprising a 3′ transposon end sequence, a 5′ adaptor sequence, and a targeting oligonucleotide coated with a recombinase, wherein the targeting oligonucleotide can bind to one or more nucleic acid sequences of interest; and a second transposon comprising a 5′ transposon end sequence, wherein the 5′ transposon end sequence is complementary to the 3′ transposon end sequence.
1. Targeting Oligonucleotides

A targeting oligonucleotide can be any type of nucleic that has affinity for one or more nucleic acid sequences of interest in a target nucleic acid. In some embodiments, a targeting oligonucleotide can hybridize to a target nucleic acid based on complementary sequences to those comprised in the target nucleic acid.

In some embodiments, a targeting oligonucleotide comprises a nucleic acid sequence that is fully or partially complementary to one or more sequence comprised in a target nucleic acid. In some embodiments, the sequence of the targeting oligonucleotide is fully or partially complementary with the one or more nucleic acid sequences of interest.

In some embodiments, a targeting oligonucleotide is 80%, 85%, 90%, 95%, 97%, 99%, or 100% complementary to sequence comprised in a target nucleic acid.

One skilled in the art could use any number of databases of sequences to develop a targeting oligonucleotide to bind to nucleic acid sequence of interest in a target nucleic acid. For example, one skilled in the art could choose a nucleic acid sequence of interest in a given gene and develop a targeting oligonucleotide complementary to the sequence of interest. In this way, the transposome complex would be targeted to the given gene.

In some embodiments, one or more targeting oligonucleotide are linked to the 5′ end of the adaptor sequence. In some embodiments, one or more targeting oligonucleotide are linked directly to the 5′ end of the adaptor sequence. In some embodiments, one or more targeting oligonucleotide are linked via a linker to the 5′ end of the adaptor sequence. In some embodiments, the linker is an oligonucleotide linker. In some embodiments, the linker is a non-oligonucleotide linker. In some embodiments, the 5′ end of the adaptor sequence and the targeting oligonucleotide are both biotinylated and linked via streptavidin.
2. Recombinases

Recombinases can mediate strand invasion of a nucleic acid. This strand invasion may be invasion of a recombinase into a double-stranded nucleic acid, such as double-stranded target DNA.

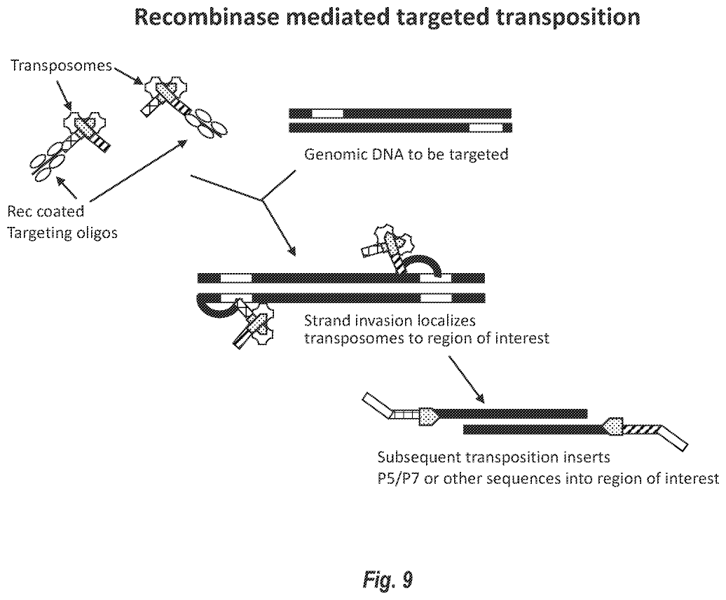

By coating a targeting oligonucleotide with a recombinase, these coated oligonucleotides can mediate strand invasion of the double-stranded nucleic acid followed by binding of the targeting oligonucleotide to one or more nucleic acid sequences of interest. Recombinase-mediated insertion of an oligonucleotide into a double-stranded target nucleic acid has been documented in Strand Invasion Based Amplification (SIBA, See, for example, Hoser et al. PLoS ONE 9(11): e112656). The recombinase can dissociate duplex regions of the double-stranded nucleic acid to allow binding of the targeting oligonucleotide to a single-stranded region of the target nucleic acid. As shown in FIG. 9, binding of recombinase-coated targeting oligonucleotides can localize transposomes to a region of interest in the target nucleic acid.

In some embodiments, the recombinase is UVSX, Rec233, or RecA.

F. Targeted Transposome Complexes Comprising a Catalytically Inactive Endonuclease

Described herein are targeted transposome complexes, wherein the complexes comprise a catalytically inactive endonuclease. In some embodiments, the catalytically inactive endonuclease serves to target the transposome complex

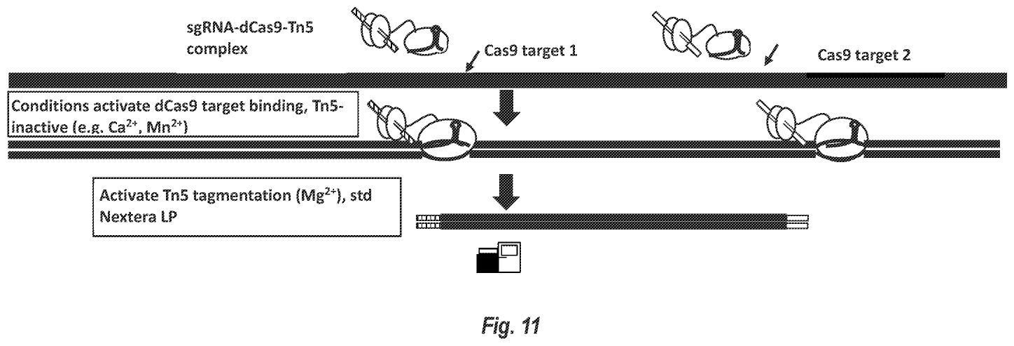

In some embodiments, a targeted transposome complex comprises a catalytically inactive endonuclease. As used herein, “catalytically inactive endonucleases” are endonucleases that can bind nucleic acid but do not mediate cleavage (this can mean that the endonuclease does not have any cleavage activity or it may mean that the endonuclease has only minimal cleavage activity such that the amount of nucleic acid lost to cleavage does not substantially interfere with the tagmentation). A catalytically inactive endonuclease may also be referred to as a deactivated endonuclease (such as a “dCas” protein). An exemplary catalytically inactive endonuclease is dCas9, as shown in FIG. 11. Normally, an endonuclease can bind to a nucleic acid and that mediate cleavage. Thus, a catalytically inactive endonuclease is one that retains nucleic acid binding function, without having cleavage activity. Catalytically inactive endonucleases can be used to target transposome complexes to one or more nucleic acid sequences of interest in a target nucleic acid. Representative catalytically inactive Cas9 proteins include those disclosed in U.S. Ser. No. 10/457,969, which is incorporated herein in its entirety.

In some embodiments, a targeted transposome complex comprises a transposase; a first transposon comprising a 3′ transposon end sequence, a 5′ adaptor sequence, and a catalytically inactive endonuclease associated with a guide RNA, wherein the guide RNA can direct endonuclease binding to one or more nucleic acid sequences of interest; and a second transposon comprising the complement of the transposon end sequence.

As used herein, a “guide RNA” is an RNA sequence that confers specificity to an endonuclease binding to a target nucleic acid. A catalytically inactive endonuclease can be targeted to one or more nucleic acid sequence of interest by a guide RNA.

A range of guide RNAs can be used with a catalytically inactive endonuclease. In some embodiments, a guide RNA comprises a trans-activating CRISPR RNA (tracrRNA) and a CRISPR RNA (crRNA). In some embodiments, a guide RNA only comprises a tracrRNA. In some embodiments, the guide RNA is a single guide RNA (or sgRNA) comprising both a tracrRNA and a crRNA.

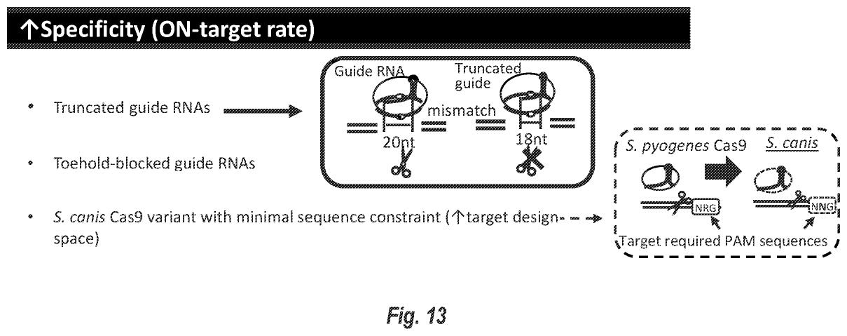

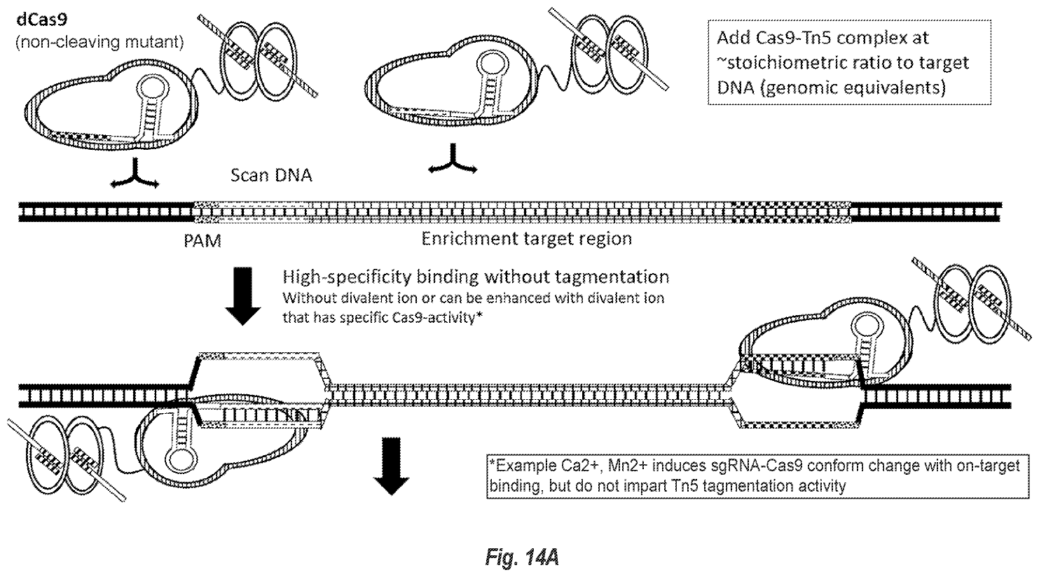

One skilled in the art can develop guide RNAs with specificity to bind to one or more sequences of interest using one of the numerous design tools available (such as those available from Synthego or Benchling). Selection of a guide RNA is also based on the presence of protospacer adjacent motifs (PAMs) within the target nucleic acid; however, endonucleases with minimal PAM specificity have been described (as shown in FIG. 13) that allow greater flexibility in designed guide RNAs.

As described herein, a single guide RNA sequence may be comprised in an oligonucleotide also comprising a transposon. Development of such oligonucleotides could be performed using standard molecular biology techniques.

In some embodiments, the catalytically inactive endonuclease is associated with the transposase. In some embodiments, the catalytically inactive endonuclease is linked to the transposase. In some embodiments, the catalytically inactive endonuclease is linked directly or indirectly to the transposase.

In some embodiments, the transposase and the catalytically inactive endonuclease are comprised in a CRISPR-associated transposase. As used herein, a “CRISPR-associated transposase” refers to a multi-protein complex comprising an endonuclease and a transposase.

Other systems wherein Tn7-like transposons have co-opted nuclease deficient CRISPR-Cas systems to generate a CRISPR-associated transposase have also been described (See Klompe et al., Nature 571:219-225 (2019)). A targeted transposome described herein may comprise any type of CRISPR-Cas system.

A catalytically inactive endonuclease can also be linked to a transposase in a number of different ways. In some embodiments, the catalytically inactive endonuclease is linked to the 5′ end of the transposase. In some embodiments, the catalytically inactive endonuclease is linked to the 3′ end of the transposase. In some embodiments, the transposase is linked to the 5′ end of the catalytically inactive endonuclease. In some embodiments, the transposase is linked to the 3′ end of the catalytically inactive endonuclease.

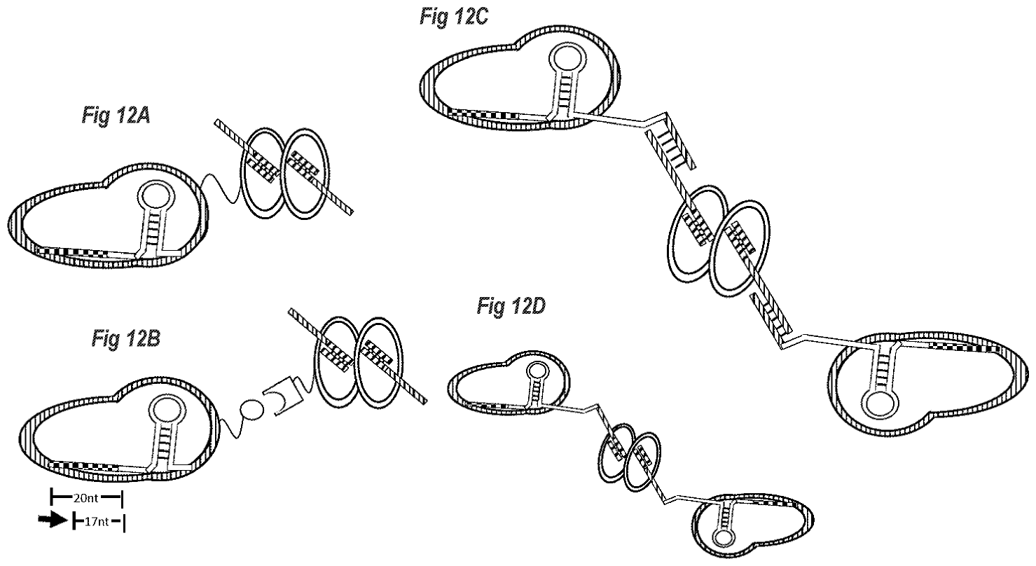

In some embodiments, the catalytically inactive endonuclease and transposase are comprised in a fusion protein, as shown in FIG. 12A. By a fusion protein, it is meant that the catalytically inactive endonuclease and transposase are comprised in a single protein. In some embodiments, the fusion protein comprising the catalytically inactive endonuclease and transposase are expressed as a single protein using a nucleic acid construct is expressed by a host cell.

In some embodiments, the catalytically inactive and transposase are directly linked. In some embodiments, the catalytically inactive and transposase are linked via a linker.

In some embodiments, the catalytically inactive endonuclease and transposase are comprised in separate proteins. In some embodiments, the catalytically inactive endonuclease and transposase are expressed as separate proteins in a host cell.

In some embodiments, the separate catalytically inactive endonuclease and transposase can associate together via pairing of binding partners, wherein a first binding partner is bound to the catalytically inactive endonuclease and a second binding partner is bound to the transposase. In some embodiments, the binding partners are biotin and streptavidin/avidin, as shown in FIG. 12B.

In some embodiments, the sgRNA is comprised in an oligonucleotide comprising the first and/or second transposon. In some embodiments, the oligonucleotide comprises a 5′ single guide RNA and a 3′ first and/or second transposon. In some embodiments, the sgRNA and the first and/or second transposon are associated with each other via pairing of complementary sequences (FIG. 12C). In some embodiments, the sgRNA and the first and/or second transposon are comprised in separate oligonucleotides. In some embodiments, the sgRNA is comprised in a contiguous sgRNA-transferred strand oligonucleotide (FIG. 12D)

A number of different means to increase the specificity of the catalytically inactive endonuclease are shown in FIGS. 12A-12D and FIG. 13. Any means of increasing the specificity of catalytically inactive endonucleases can also be used to increase the specificity of catalytically active endonucleases.

In some embodiments, the single guide RNA comprises less than 20 nucleotides (such as the embodiment with 17 nucleotides in FIG. 12B or embodiment with 18 nucleotides in FIG. 13). Such a single guide RNA comprising less than 20 nucleotides may be referred to as a truncated guide RNA. In some embodiments, the single guide RNA sequence comprises 15, 16, 17, 18, or 19 nucleotides. Shorter single guide RNAs reduce the possibility of single guide RNA binding to sequences in the target nucleic acid that are not fully or highly complementary to the sequence of sgRNA.

In some embodiments, the single guide RNA comprises a hairpin secondary structure (Kocak et al., Nat Biotechnol. 37(6): 657-666 (2019)). In some embodiments, a hairpin secondary structure is used to block binding to a target nucleic acid in the absence of a trigger strand, such as a toehold-blocked guide RNA (Siu et al. Nat Chem Biol 15(3):217-220 (2019)).

In some embodiments, the catalytically inactive endonuclease is a Cas9 protein (which may be referred to as a deactivated Cas9 or dCas9). A wide variety of different Cas9 proteins may be comprised in targeted transposome complexes described herein. Further, one skilled in the art would be aware of catalytic domains of endonucleases and could design a mutation to generate catalytically inactive endonuclease from a wildtype endonuclease (See Maeder et al., Nat Methods 10(10): 977-979 (2013)). Such a designed catalytically inactive endonuclease could be tested to confirm its lack of cleavage activity.

In some embodiments, the Cas9 protein is a Streptococcus canis Cas9, as shown in FIG. 13. In some embodiments, the Streptococcus canis Cas9 has minimal sequence constraint (See Chatterjee et al., Sci. Adv. 4:eaau0766 (2018)). In some embodiments, the Streptococcus canis Cas9 has reduced requirement for a specific protospacer adjacent motif (PAM) in proximity to a sequence in the target nucleic acid that can bind to the guide RNA. For example, a Streptococcus canis Cas9 may require a NNG PAM sequence, in lieu of a NRG PAM sequence (as shown in FIG. 13), which reduces requirement for a specific PAM and increases the ability to choose sequences of interest for binding to the guide RNA. The lower sequence constraint of an endonuclease with minimal sequence constraint can allow for improved target design space, since it lowers the requirement for a specific PAM sequence in proximity to the sequence of interest in a target nucleic acid.

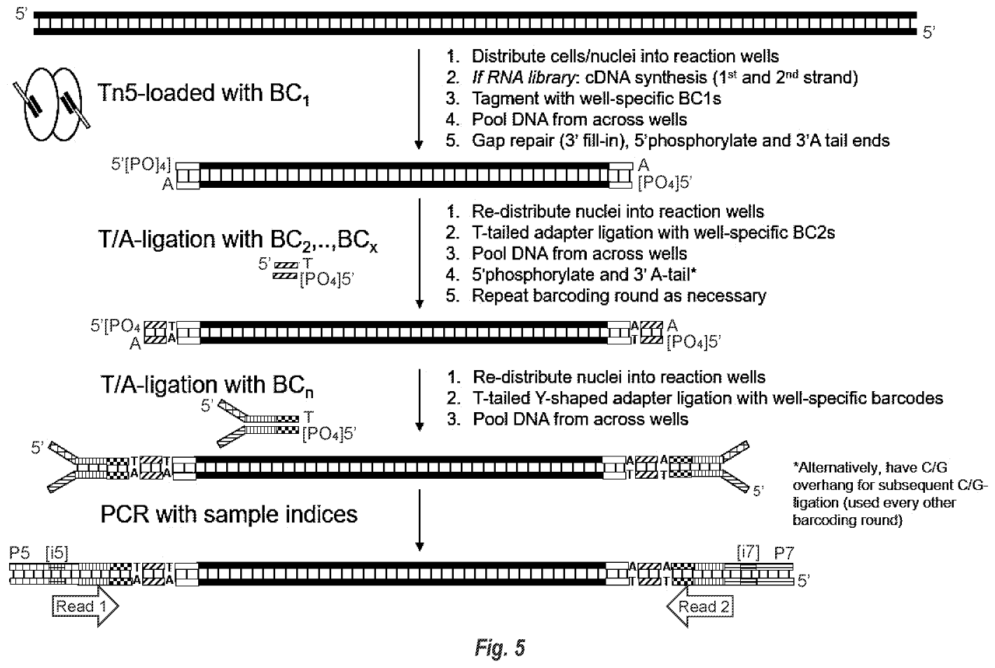

In some embodiments, the CRISPR-associated transposase is from cyanobacteria Scytonema hofmanni (ShCAST). ShCAST is a 4-protein system for RNA-directed (sgRNA) DNA-transposition mediated by Tn7-like transposase subunits and the type V-K CRISPR effector (Cas12k) (See Strecker et al., Science. 365(6448): 48-53 (2019), including the embodiment shown in FIG. 5 of Strecker, all of which are incorporated by reference for the teachings regarding ShCAST). It has been suggested that these systems comprising Tn7-like transposons and CRISPR-Cas systems might have hijacked CRISPR effectors to generate R-loops in target sites and to facilitate the spread of transposons via plasmids and phages. ShCAST can lead to insertion into unique sites in a target nucleic acid via RNA-guided Tn7-like transposons. Thus, in some embodiments, a targeted transposome complex comprises a catalytically inactive endonuclease and a transposase within a ShCAST to enable targeted transposition.
1. Targeted Transposome Complexes Comprising a Cas Endonuclease

In some embodiments, a targeted transposome complex comprises a Cas endonuclease.

As used herein, terms such as “CRISPR-Cas system,” “Cas-gRNA ribonucleoprotein,” and Cas-gRNA RNP refer to an enzyme system including a guide RNA (gRNA) sequence that includes an oligonucleotide sequence that is complementary or substantially complementary to a sequence within a target nucleic acid, and a Cas protein. CRISPR-Cas systems may generally be categorized into three major types which are further subdivided into ten subtypes, based on core element content and sequences; see, e.g., Makarova et al., “Evolution and classification of the CRISPR-Cas systems,” Nat Rev Microbiol. 9(6): 467-477 (2011). Cas proteins may have various activities, e.g., nuclease activity. Thus, CRISPR-Cas systems provide mechanisms for targeting a specific sequence (e.g., via the gRNA) as well as certain enzyme activities upon the sequence (e.g., via the Cas protein).

A Type I CRISPR-Cas system may include Cas3 protein with separate helicase and DNase activities. For example, in the Type 1-E system, crRNAs are incorporated into a multisubunit effector complex called Cascade (CRISPR-associated complex for antiviral defense), which binds to the target DNA and triggers degradation by the Cas3 protein; see, e.g., Brouns et al., “Small CRISPR RNAs guide antiviral defense in prokaryotes,” Science 321(5891): 960-964 (2008); Sinkunas et al., “Cas3 is a single-stranded DNA nuclease and ATP-dependent helicase in the CRISPR-Cas immune system,” EMBO J 30:1335-1342 (2011); and Beloglazova et al., “Structure and activity of the Cas3 HD nuclease MJ0384, an effector enzyme of the CRISPR interference, EMBO J 30:4616-4627 (2011). Type II CRISPR-Cas systems include the signature Cas9 protein, a single protein (about 160 KDa) capable of generating crRNA and cleaving the target DNA. The Cas9 protein typically includes two nuclease domains, a RuvC-like nuclease domain near the amino terminus and the HNH (or McrA-like) nuclease domain near the middle of the protein. Each nuclease domain of the Cas9 protein is specialized for cutting one strand of the double helix; see, e.g., Jinek et al., “A programmable dual-RNA-guided DNA endonuclease in adaptive bacterial immunity, Science 337(6096): 816-821 (2012). Type III CRISPR-Cas systems include polymerase and RAMP modules. Type III systems can be further divided into sub-types III-A and III-B. Type III-A CRISPR-Cas systems have been shown to target plasmids, and the polymerase-like proteins of Type III-A systems are involved in the cleavage of target DNA; see, e.g., Marraffini et al., “CRISPR interference limits horizontal gene transfer in Staphylococci by targeting DNA,” Science 322(5909):1843-1845 (2008). Type III-B CRISPR-Cas systems have also been shown to target RNA; see, e.g., Hale et al., “RNA-guided RNA cleavage by a CRISPR-RNA-Cas protein complex,” Cell 139(5): 945-956 (2009). CRISPR-Cas systems include engineered and/or programmed nuclease systems derived from naturally accruing CRISPR-Cas systems. CRISPR-Cas systems may include engineered and/or mutated Cas proteins. CRISPR-Cas systems may include engineered and/or programmed guide RNA.

In some embodiments, the Cas protein in one of the present Cas-gRNA RNPs may include Cas9 or other suitable Cas that may cut the target nucleic acid at the sequence to which the gRNA is complementary, in a manner such as described in the following references, the entire contents of each of which are incorporated by reference herein: Nachmanson et al., “Targeted genome fragmentation with CRISPR/Cas9 enables fast and efficient enrichment of small genomic regions and ultra-accurate sequencing with low DNA input (CRISPR-DS),” Genome Res. 28(10): 1589-1599 (2018); Vakulskas et al., “A high-fidelity Cas9 mutant delivered as a ribonucleoprotein complex enables efficient gene editing in human hematopoietic stem and progenitor cells,” Nature Medicine 24: 1216-1224 (2018); Chatterjee et al., “Minimal PAM specificity of a highly similar SpCas9 ortholog,” Science Advances 4(10): eaau0766, 1-10 (2018); Lee et al., “CRISPR-Cap: multiplexed double-stranded DNA enrichment based on the CRISPR system,” Nucleic Acids Research 47(1): 1-13 (2019). Isolated Cas9-crRNA complex from the S. thermophilus CRISPR-Cas system as well as complex assembled in vitro from separate components demonstrate that it binds to both synthetic oligodeoxynucleotide and plasmid DNA bearing a nucleotide sequence complementary to the crRNA. It has been shown that Cas9 has two nuclease domains-RuvC- and HNH-active sites/nuclease domains, and these two nuclease domains are responsible for the cleavage of opposite DNA strands. In some examples, the Cas9 protein is derived from Cas9 protein of S. thermophilus CRISPR-Cas system. In some examples, the Cas9 protein is a multi-domain protein having about 1,409 amino acids residues.

In other embodiments, the Cas may be engineered so as not to cut the target nucleic acid at the sequence to which the gRNA is complementary to prepared a deactivated Cas (dCas), e.g., in a manner such as described in the following references, the entire contents of each of which are incorporated by reference herein: Guilinger et al., “Fusion of catalytically inactive Cas9 to Fok1 nuclease improves the specificity of genome modification,” Nature Biotechnology 32: 577-582 (2014); Bhatt et al., “Targeted DNA transposition using a dCas9-transposase fusion protein,” https://doi.org/10.1101/571653, pages 1-89 (2019); Xu et al., “CRISPR-assisted targeted enrichment-sequencing (CATE-seq),” available at URL www.biorxiv.org/content/10.1101/672816v1, 1-30 (2019); and Tijan et al., “dCas9-targeted locus-specific protein isolation method identifies histone gene regulators,” PNAS 115(12): E2734-E2741 (2018). Cas that lacks nuclease activity may be referred to as deactivated Cas (dCas). In some embodiments, the dCas may include a nuclease-null variant of the Cas9 protein, in which both RuvC- and HNH-active sites/nuclease domains are mutated. A nuclease-null variant of the Cas9 protein (dCas9) binds to double-stranded DNA, but does not cleave the DNA. Another variant of the Cas9 protein has two inactivated nuclease domains with a first mutation in the domain that cleaves the strand complementary to the crRNA and a second mutation in the domain that cleaves the strand non-complementary to the crRNA. In some embodiments, the Cas9 protein has a first mutation D10A and a second mutation H840A.

In some embodiments, the Cas protein comprises a Cascade protein. Cascade complex in E. coli recognizes double-stranded DNA (dsDNA) targets in a sequence-specific manner. E. coli Cascade complex is a 405-kDa complex including five functionally essential CRISPR-associated (Cas) proteins (CasA1B2C6D1E1, also called Cascade protein) and a 61-nucleotide crRNA. The crRNA guides Cascade complex to dsDNA target sequences by forming base pairs with the complementary DNA strand while displacing the noncomplementary strand to form an R-loop. Cascade recognizes target DNA without consuming ATP, which suggests that continuous invader DNA surveillance takes place without energy investment; see, e.g., Matthijs et al., “Structural basis for CRISPR RNA-guided DNA recognition by Cascade,” Nature Structural & Molecular Biology 18(5): 529-536 (2011). In some embodiments, the Cas protein includes a Cas3 protein. Illustratively, E. coli Cas3 may catalyze ATP-independent annealing of RNA with DNA forming R-loops, and hybrid of RNA base-paired into duplex DNA. Cas3 protein may use gRNA that is longer than that for Cas9; see, e.g., Howard et al., “Helicase disassociation and annealing of RNA-DNA hybrids by Escherichia coli Cas3 protein,” Biochem J. 439(1): 85-95 (2011). Such longer gRNA may permit easier access of other elements to the target DNA, e.g., access of a primer to be extended by polymerase. Another feature provided by Cas3 protein is that Cas3 protein does not require a PAM sequence as may Cas9, and thus provides more flexibility for targeting desired sequence. R-loop formation by Cas3 may utilize magnesium as a co-factor; see, e.g., Howard et al., “Helicase disassociation and annealing of RNA-DNA hybrids by Escherichia coli Cas3 protein,” Biochem J. 439(1): 85-95 (2011). It will be appreciated that any suitable cofactors, such as cations, may be used together with the Cas proteins used in the present compositions and methods.

It also should be appreciated that any CRISPR-Cas systems capable of disrupting the double stranded polynucleotide and creating a loop structure may be used. For example, the Cas proteins may include, but not limited to, Cas proteins such as described in the following references, the entire contents of each of which are incorporated by reference herein: Haft et al., “A guild of 45 CRISPR-associated (Cas) protein families and multiple CRISPR/Cas subtypes exist in prokaryotic genomes,” PLoS Comput Biol. 1(6): e60, 1-10 (2005); Zhang et al., “Expanding the catalog of cas genes with metagenomes,” Nucl. Acids Res. 42(4): 2448-2459 (2013); and Strecker et al., “RNA-guided DNA insertion with CRISPR-associated transposases,” Science 365(6448): 48-53 (2019) in which the Cas protein may include Cas12k. Some these CRISPR-Cas systems may utilize a specific sequence to recognize and bind to the target sequence. For example, Cas9 may utilize the presence of a 5′-NGG protospacer-adjacent motif (PAM).

CRISPR-Cas systems may also include engineered and/or programmed guide RNA (gRNA). As used herein, the terms “guide RNA” and “gRNA” (and sometimes referred to in the art as single guide RNA, or sgRNA) is intended to mean RNA including a sequence that is complementary or substantially complementary to a region of a target DNA sequence and that guides a Cas protein to that region. A guide RNA may include nucleotide sequences in addition to that which is complementary or substantially complementary to the region of a target DNA sequence. Methods for designing gRNA are well known in the art, and nonlimiting examples are provided in the following references, the entire contents of each of which are incorporated by reference herein: Stevens et al., “A novel CRISPR/Cas9 associated technology for sequence-specific nucleic acid enrichment,” PLoS ONE 14(4): e0215441, pages 1-7 (2019); Fu et al., “Improving CRISPR-Cas nuclease specificity using truncated guide RNAs, Nature Biotechnology 32(3): 279-284 (2014); Kocak et al., “Increasing the specificity of CRISPR systems with engineered RNA secondary structures,” Nature Biotechnology 37: 657-666 (2019); Lee et al., “CRISPR-Cap: multiplexed double-stranded DNA enrichment based on the CRISPR system,” Nucleic Acids Research 47(1): el, 1-13 (2019); Quan et al., “FLASH: a next-generation CRISPR diagnostic for multiplexed detection of antimicrobial resistance sequences,” Nucleic Acids Research 47(14): e83, 1-9 (2019); and Xu et al., “CRISPR-assisted targeted enrichment-sequencing (CATE-seq),” https://doi.org/10.1101/672816, 1-30 (2019).

In some embodiments, gRNA includes a chimera, e.g., CRISPR RNA (crRNA) fused to trans-activating CRISPR RNA (tracrRNA). Such a chimeric single-guided RNA (sgRNA) is described in Jinek et al., “A programmable dual-RNA-guided endonuclease in adaptive bacterial immunity,” Science 337 (6096): 816-821 (2012). The Cas protein may be directed by a chimeric sgRNA to any genomic locus followed by a 5′-NGG protospacer-adjacent motif (PAM). In one nonlimiting example, crRNA and tracrRNA may be synthesized by in vitro transcription, using a synthetic double-stranded DNA template including the T7 promoter. The tracrRNA may have a fixed sequence, whereas the target sequence may dictate part of the crRNA's sequence. Equal molarities of crRNA and tracrRNA may be mixed and heated at 55° C. for 30 seconds. Cas9 may be added at the same molarity at 37° C. and incubated for 10 minutes with the RNA mix. A 10- to 20-fold molar excess of the resulting Cas9-gRNA RNP then may be added to the target DNA. The binding reaction may occur within 15 minutes. Other suitable reaction conditions readily may be used.
2. Targeted Transposome Complexes Comprising ShCAST

In some embodiments, a targeted transposome complex is comprised in a ShCAST.

Some examples herein provide a composition that includes a target nucleic acid (such as a double-stranded nucleic acid) comprising one or more sequence of interest. The composition may include a plurality of complexes each including an ShCAST (Scytonema hofmanni CRISPR associated transposase) coupled to guide RNA (gRNA). The ShCAST may have an amplification adapter coupled thereto. Each of the complexes may be hybridized to a corresponding one of the subsequences in the target nucleic acid (such as one or more nucleic acid sequences of interest). Such complexes are disclosed in U.S. Provisional Application Nos. U.S. 63/162,775 and U.S. 63/163,381, each of which are incorporated by reference in their entirety herein.

In some embodiments, a composition comprises (1) a target nucleic acid comprising one or more nucleic acid sequences of interest and (2) a plurality of targeted transposome complexes described herein each comprising an ShCAST coupled to gRNA, wherein the ShCAST has an amplification adapter coupled thereto, and wherein each of the targeted transposome complexes is hybridized to a nucleic acid sequence of interest.

In some embodiments, ShCAST comprises a catalytically inactive endonuclease (such as Cas12K) and a transposase (such as Tn5). In some aspects, cleavage of a nucleic acid by ShCAST may be considered a two-step process, with 1) binding to a nucleic acid based on association of the catalytically inactive endonuclease to a gRNA bound to one or more sequences of interest and 2) cleavage by the transposase. In some embodiments, limiting non-specific binding of the transposase to the nucleic acid increases the frequency of preparation of targeted fragments (i.e., fragments generated from cleavage after association of the catalytically inactive endonuclease with the gRNA).

In some embodiments, the composition further includes a fluid having a condition promoting hybridization of the complexes to the subsequences and inhibiting binding of the transposases. In some examples, the condition of the fluid comprises absence of a sufficient amount of magnesium ions for activity of the transposases.

By inhibiting binding by the transposase, cleavage by the ShCAST is limited to sites where the Cas12K comprised in the ShCAST has associated with a gRNA bound to sequences of interest in a nucleic acid. In this way, non-specific cleavage (due to non-specific binding of the transposase to the nucleic acid) is limited, and most cleavage of the nucleic acid is at sites within or near the sequence of interest.

In some embodiments, a condition for limiting binding of the transposase comprised in the complex is a magnesium concentration of 15 mM or lower and/or with a concentration of transposase of 50 nM or lower. Such compositions that inhibit binding of transposases may serve to inhibit non-specific cleavage by transposases comprised in the ShCAST, with most cleavage occurring based on binding of the CasK12 to gRNAs bound to sequences of interest in the nucleic acid.

In some examples, the composition further includes a fluid having a condition promoting activity of the transposases, and in which the transposases add the amplification adapters to locations in the target nucleic acid. In some examples, the condition of the fluid comprises the presence of a sufficient amount of magnesium ions for activity of the transposases. Such embodiments that promote activity of transposases may be those for preparing fragments at or near sequences of interest bound by gRNAs, such as by tagmentation. Such conditions could be a magnesium concentration of 15 mM or higher.

In some embodiments, the ShCAST comprises Cas12K. In some examples, the transposase comprises Tn5 or a Tn7-like transposase. In some embodiments, the adapter comprises at least one of a P5 adapter and a P7 adapter. In some embodiments, the target nucleic acid comprises double-stranded DNA.

In some examples, at least one of the gRNA and the transposase is biotinylated. The composition further may include a streptavidin-coated bead to which the at least one of the gRNA and transposase that is biotinylated is coupled.

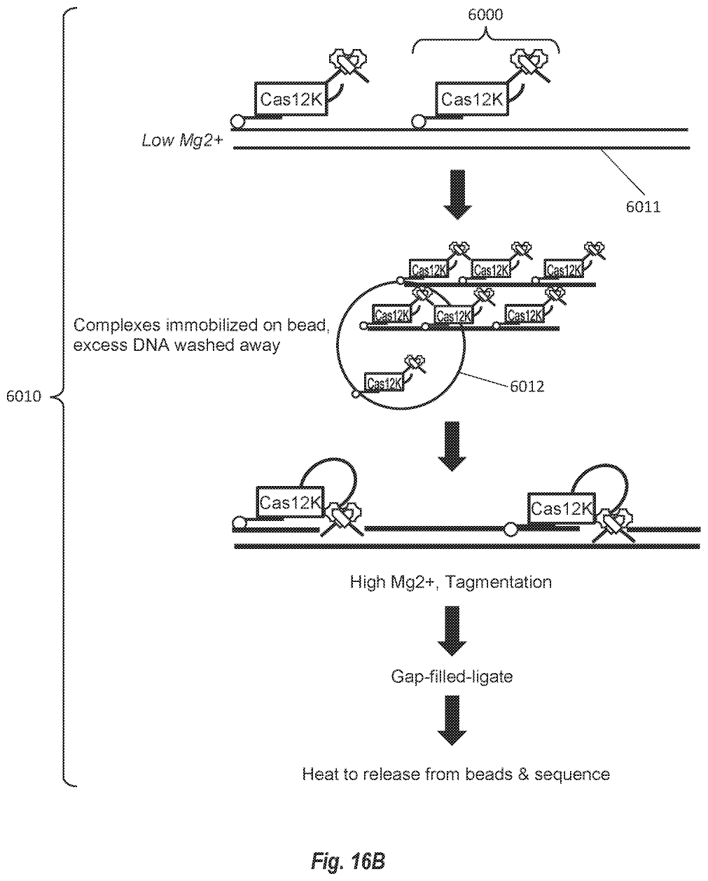

For example, FIGS. 16A and 16B schematically illustrate example compositions and operations in a process for ShCAST (Scytonema hofmanni CRISPR associated transposase) targeted library preparation and enrichment. ShCAST 6000 includes Cas12k 6001 and a Tn7-like transposase 6002 that is capable of inserting DNA 6003 into specific sites in the E. coli genome using RNA guides 6004. Some examples provided herein utilize ShCAST or a modified version of ShCAST incorporating a Tn5 transposase (ShCAST-Tn5) for targeted amplification of specific genes. As such, library preparation and enrichment steps are combined, thus simplifying and improving the efficiency of the target library sequencing workflow, and facilitating automation.

Illustratively, gRNA 6004 may be designed to target specific genes (sequences), and the spacing of the gRNAs may control the insert size. In some examples, the gRNA 6004 and/or the ShCAST/ShCAST-Tn5 6002 may be coupled to a tag 6005, e.g., may be biotinylated. In a manner such as illustrated in FIG. 16A, gRNAs 6004 and transposable elements with adapters 6003 (e.g., Illumina adapters) may be loaded onto the transposase 6002 of ShCAST, resulting in complex 6000. In a manner such as illustrated in process flow 6010 of FIG. 16B, the resulting ShCAST/ShCAST-Tn5 complexes 6000 may be mixed with genomic DNA (target nucleic acid) 6011 under fluidic conditions (e.g., low or no magnesium) that inhibit tagmentation, while allowing the complexes to bind to respective sequences in the target DNA The complexes then may be isolated using substrates coupled to tag partners, such as streptavidin beads 6012 to which the tagged (e.g., biotinylated) gRNA and/or ShCAST/ShCAST-Tn5 becomes coupled. Any unbound DNA may be washed away, e.g., to reduce or minimize off-target tagmentation. Then the fluidic conditions may be altered (e.g., sufficiently increasing magnesium) to promote tagmentation. A gap-fill-ligation step followed by heat dissociation may be used to release the library from beads in preparation for sequencing.

Note that in compositions and operations such as illustrated in FIGS. 16A and 16B, the transposase portion 6002 of the complex 6000 may be able to randomly insert into the DNA. Such insertion may be inhibited or minimized by mixing the ShCAST/ShCAST-Tn5 complexes with the genomic DNA under fluidic conditions (e.g., low or no magnesium) that inhibit tagmentation, thus allowing targets to be bound.

In some embodiments, methods are designed to limit off-target tagmentation. In some embodiments, low concentrations of Tn5 during a method of targeted transposition with ShCAST limits off-target tagmentation. In some embodiments, low concentrations of Tn5 limit how much ShCAST is bound non-specifically to nucleic acid.

In some embodiments, a gRNA targets binding of the ShCAST (and therefore the transposase) at one or more loci of interest within the target nucleic acid, which enables the user to generate amplifiable PCR products with both forward and reverse primers. In some embodiments, different gRNA bind to different sequences at a locus of interest, i.e., different gRNA bind to more than one sequence of interest within a locus of interest. Such a loci of interest may be sequences within or in close proximity to a gene of interest, for example.

Fragments generated using the present methods require tagmentation by two transposome complexes to all for preparation of fragments with appropriate adapters at both ends. If a fragment is generated using one targeted transposome complex that is targeted to a locus of interest (by a gRNA) and the other transposome complex binds randomly, the fragment is likely to be too large to be amplified properly using the present methods. In some embodiments, when the transposase concentration is very low, the chances of it binding randomly across the genome next to another Tn5 in close enough proximity to generate a amplifiable/sequenceable fragment is low. Alternatively, binding and cleavage by ShCAST may be performed at a low temperature (such as below 37° C.). Accordingly, a fragment generated via off-target binding and tagmentation with a ShCAST will likely not be an amplifiable PCR product. Only when transposases are clustered in relatively close proximity (as with ShCAST complexes targeted using gRNAs designed to target a loci of interest) will fragments be generated that can undergo PCR enrichment.

For further details regarding ShCAST, including the Cas12k and Tn7 therein, see Strecker et al., Science. 365(6448): 48-53 (2019), which is incorporated by reference herein in its entirety.

G. Targeted Transposomes Comprising Zinc Finger DNA-Binding Domains

In some embodiments, a targeted transposome complex comprises a zinc finger DNA-binding domain. This zinc finger DNA-binding domain may serve to target the transposome complex to a sequence of interest in a target nucleic acid.

In some embodiments, a zinc finger DNA-binding domain is designed to bind to one or more sequences of interest in a target nucleic acid. Means of designing zinc finger DNA-binding domains to bind particular sequences are well-known in the field (See Wei et al., BMC Biotechnology 8:28 (2008)).

In some embodiments, a targeted transposome complex comprises a transposase, a first transposon comprising a 3′ transposon end sequence; a 5′ adaptor sequence; and a zinc finger DNA-binding domain, wherein the zinc finger DNA-binding domain can bind to one or more nucleic acid sequences of interest; and a second transposon comprising the complement of the transposon end sequence.

In some embodiments, the complex comprises a zinc finger DNA-binding domain array. As used herein, a “zinc finger DNA-binding array” is a domain comprises more than one zinc finger DNA-binding domain.

In some embodiments, the zinc finger DNA-binding domain is associated with the transposase. In some embodiments, the zinc finger DNA-binding domain is linked to the transposase.

In some embodiments, the zinc finger DNA-binding domain is linked to the 5′ end of the transposase. In some embodiments, the zinc finger DNA-binding domain is linked to the 3′ end of the transposase. In some embodiments, the transposase is linked to the 5′ end of the zinc finger DNA-binding domain. In some embodiments, the transposase is linked to the 3′ end of the zinc finger DNA-binding domain. In some embodiments, the zinc finger DNA-binding domain and transposase are comprised in a fusion protein.

In some embodiments, the zinc finger DNA-binding domain and transposase are linked via a linker.

In some embodiments, the zinc finger DNA-binding domain and transposase are comprised in separate proteins. In some embodiments, the separate zinc finger DNA-binding domain and transposase can associate together via pairing of binding partners, wherein a first binding partner is bound to the catalytically inactive endonuclease and a second binding partner is bound to the transposase.

### II. Kits or Compositions Comprising Targeted Transposome

A variety of kits or compositions may comprise targeted transposome complexes.

In some embodiments, a kit or composition comprises a first transposome complex that is a targeted transposome complex and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence.

In some embodiments, a first transposome complex that is a targeted transposome complex comprises a targeting oligonucleotide coated with a recombinase. In some embodiments, a kit or composition comprises two transposome complexes that are each a targeted transposome complex, wherein the two targeted transposome complexes comprises different targeting oligonucleotides.

In some embodiments, a kit or composition comprises two transposome complexes that are each a targeted transposome complex, wherein the two targeted transposome complexes comprises different guide RNAs.

In some embodiments, a kit or composition comprises two transposome complexes that are each a targeted transposome complex, wherein the two targeted transposome complexes comprise different zinc finger DNA-binding domains.

### III. Methods Using Targeted Transposome Complexes for Targeted Transposition

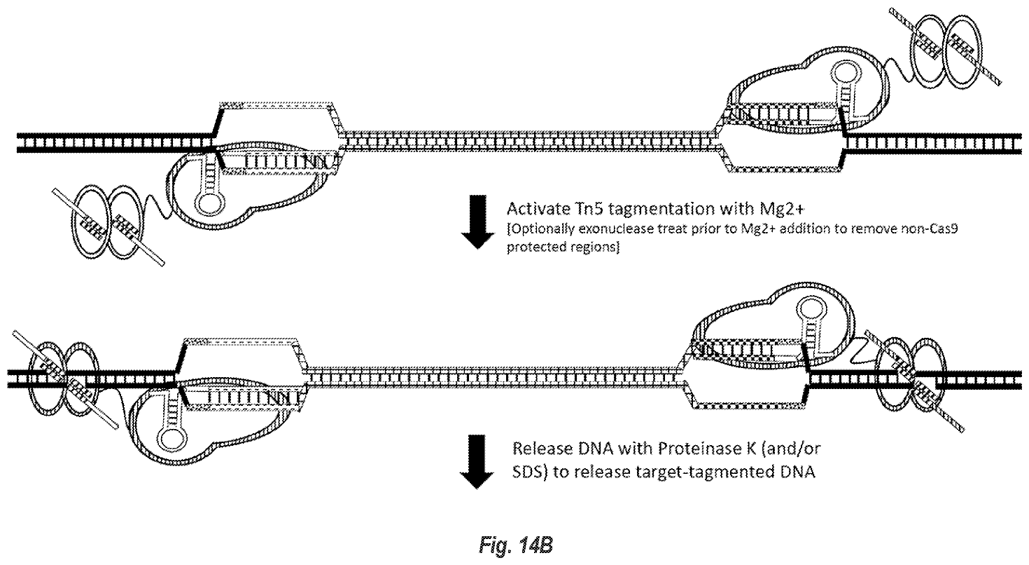

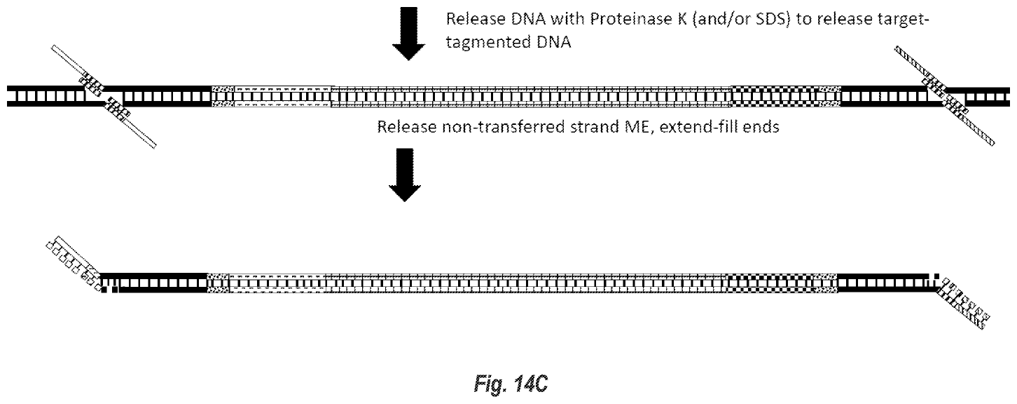

Methods using targeted transposome complexes can mediate transposition within a region of a target nucleic acid in close proximity to where the targeted transposome complex is bound to the target nucleic. In other words, targeted transposome complexes can mediate sequence-specific targeted transposition of nucleic acids. Sequence-specific transposition can be used for fragmenting a target nucleic acid and generating tagged fragments comprising a specific portion of a target nucleic acid. A representative method using targeted transposome complexes is shown in FIGS. 14A-14C, wherein the targeted transposome complex comprises a non-cleaving endonuclease mutant, such as dCas9.

Generally, transposome complexes mediate transposition by randomly binding double-stranded nucleic acids. However, for some uses, one skilled in the art may prefer to prepare libraries comprising fragments comprising a desired portion of a target nucleic acid. This desired portion may be termed an enrichment target region as shown in FIG. 14A.

A library generated via a method that increases the probability of the library comprising fragments comprising a certain portion of a target nucleic acid may be termed a “targeted library.” The present methods using targeted transposome complexes can be used to generate a targeted library. As used herein, a “non-targeted library” refers to a library comprising random fragments of the target nucleic acid (for example, a library generated with random fragments such as by standard tagmentation methods).

In some embodiments, there is higher frequency of transposition around desired sites in the target nucleic acid when using a targeted transposome. In some embodiments, a targeted library generated via the present methods may also comprise fragments comprising other portions of the target nucleic acid. In other words, a targeted library may also comprise fragments comprising other portions of the target nucleic acid.

In some embodiments, 10%, 20%, 30%, 40%, 50%, 60%, 70%, 80%, 90%, 95%, 99%, or 100% of tagged fragments comprised in a library of fragments generated via the present methods comprises fragments of the desired portions of the target nucleic acid.

In some embodiments, a library of fragments generated via the present methods using targeted transposome complexes comprise 2×, 5×, 10×, 20×, 50×, 100×, or 1000× more tagged fragments comprising the desired portions of the target nucleic acid compared a library that was not generated via targeted transposome complexes or other enrichment methods (i.e., a non-targeted or non-enriched library). In some embodiments, a non-targeted or non-enriched library may have been generated via a method using transposome complexes that randomly bind to and fragment target nucleic acid.

In some embodiments, a library of fragments generated via the present methods is enriched 2×, 5×, 10×, 20×, 50×, 100×, or 1000× for tagged fragments comprising the desired portions of the target nucleic acid. In other words, a library of fragments generated via the present methods using targeted transposome complexes may have a higher frequency of tagged fragments comprising the desired portions of the target nucleic acid, as compared to the frequency of these fragments in a non-targeted or non-enriched library.

Targeted libraries have a number of important advantages. Targeted libraries focus on regions of interest in the target nucleic acid to generate a smaller, more manageable data sets in down-stream applications, such as sequencing. Methods using targeted libraries can also reduce sequencing costs and data analysis burdens, as well as reduce turnaround time compared to methods using non-targeted libraries.

Libraries comprising selected regions of a target nucleic (“targeted libraries”) may be important for a range of applications. Generally, methods for targeted analysis of specific genes of interest (i.e., custom content), targets within genes, or mitochondrial DNA may also be amenable to the present methods for generating targeted libraries. Targeted libraries may be desired where platform outputs are limiting or when very high coverage is required. For example, targeted libraries can enable deep sequencing at high coverage levels for rare variant identification.

In some embodiments, methods using targeted transposome complexes allow use of lower concentrations of transposome complexes in relation to the amount of target nucleic acid compared to non-targeted transposome complexes. In some embodiments, the targeted transposome complexes are used at an approximately equal stoichiometry to the target DNA.

In other words, a molar excess of targeted transposome complexes may not be needed to generate a library with sufficient fragments comprising a region of interest from the target nucleic acid. In comparison, to obtain sufficient fragments in a non-targeted library (i.e., a library generation method that does not target transposome complexes to one or more nucleic acid sequences of interest) many more transposome complexes may be needed, as the fragments generated with a non-targeted library are produced randomly. Thus, with a targeted transposome, many more fragments in a library may contain a sequence of interest, which allows use of lower amounts of the targeted transposome complex and lower amounts of the target nucleic acid.

The targeted transposome complexes described herein may be used together with a non-targeted transposome complex. In some embodiments, a method of generating a library of tagged nucleic acid fragments comprises combining a sample comprising a double-stranded nucleic acid, a first transposome complex that is a targeted transposome complex, and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

Methods may also use two targeted transposome complexes.

In some embodiments, a method of generating a library of tagged nucleic acid fragments comprises combining a sample comprising a double-stranded nucleic acid, a first transposome complex that is a targeted transposome complex, and a second transposome complex that is a targeted transposome complex; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

The targeted transposome used in a method may be any of those described herein, such as those comprising a catalytically inactive endonuclease or comprising a zinc finger DNA-binding domain.

A method described herein may be designed to promote combining of a targeted transposome complex with a target nucleic before fragmenting. In some embodiments, an agent that promotes fragmenting activity of a transposase is absent or at low levels during a combining step. In some embodiments, divalent cations are absent during the combining. In some embodiments, Ca²⁺ and/or Mn²⁺ are present during the combining. In some embodiments, Ca²⁺ and/or Mn²⁺ are present during the combining, but Mg²⁺ is absent.

In some embodiments, a method further comprises adding one or more divalent cations to the sample after the combining and before the fragmenting. In some embodiments, the divalent cation is Mg²⁺.

In some embodiments, a method further comprises comprising treating the sample with an exonuclease after the combining and before the fragmenting. An exonuclease may promote degradation of single-stranded DNA. In some embodiments, a method further comprises adding Mg²⁺ after the treating sample with an exonuclease and before the fragmenting.

In some embodiments, a method comprises releasing the tagged fragments with proteinase K and/or SDS.

The present methods may be used to tag both ends of generated fragments with adaptors. This may be achieved by using methods with a first transposome complex and a second transposome complex. In some embodiments, the method incorporates different tags onto each end of fragments generating by fragmenting. In some embodiments, the 5′ adaptor sequences comprised in the first transposome complex and the second transposome complex are different.

A. Methods Using Targeted Transposome Complexes Comprising a Targeting Oligonucleotide Coated with a Recombinase

In some embodiments, methods use targeted transposome complexes comprising a targeting oligonucleotide coated with a recombinase. An exemplary embodiment is shown in FIG. 9.

In some embodiments, a method of targeted generation of 5′ tagged fragments of a target nucleic acid comprises combining a sample comprising a double-stranded nucleic acid and a transposome complexes that is a targeted transposome complex. In some embodiments, the targeted transposome complex comprises a targeting oligonucleotide coated with a recombinase. In some embodiment, strand invasion of the nucleic acid is initiated by the recombinase. In some embodiments, after strand invasion, the nucleic acid is fragmented into a plurality of fragments by the transposase, by joining the 3′ end of the first transposon to the 5′ ends of the fragments to produce a plurality of 5′ tagged fragments.

In some embodiments, a method of generating a library of tagged nucleic acid fragments comprises combining a sample comprising a double-stranded nucleic acid, a first transposome complex that is a targeted transposome complex comprising a targeting oligonucleotide coated with a recombinase, and a second transposome complex comprising a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence; initiating strand invasion of the nucleic acid by the recombinase; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

In some embodiments, a method of generating a library of tagged nucleic acid fragments comprises combining a sample comprising a double-stranded nucleic acid, a first transposome complex that is a targeted transposome complex comprising a targeting oligonucleotide coated with a recombinase, and a second transposome complex that is a targeted transposome complex comprising a targeting oligonucleotide coated with a recombinase; initiating strand invasion of the nucleic acid by the recombinase; and fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

In some embodiments, the 5′ adaptor sequences comprised in the first transposome complex and the second transposome complex are different.

In some embodiments, the targeting oligonucleotide comprised in the first transposome complex and the second transposome complex are different.

In some embodiments, the targeting oligonucleotide of the first transposome complex and the second transposome complex bind to different sequences of interest in a given region of interest in a target nucleic acid. In this way, the first transposome complex and the second transposome complex may generate fragments comprising desired sequences of interest. One skilled in the art could design targeting oligonucleotides that bind at, near, or beyond the ends of a sequence of interest to generate fragments comprising this sequence of interest. In this way, a targeted library can be generated with an increased frequency of fragments comprising the sequence of interest.

In some embodiments, the second transposome complex binds to the opposite strand of the double-stranded nucleic acid compared to the first transposome complex.

In some embodiments, initiating strand invasion of the nucleic acid by the recombinase is performed in the presence of a recombinase loading factor.

In some embodiments, the recombinase loading factor is removed or inactivated before fragmenting.

In some embodiments, initiating strand invasion occurs via displacement loop formation.

In some embodiments, strand invasion is initiated within 40, 30, 20, 15, 10, or 5 bases of the binding site of the targeting oligonucleotide to the one or more sequences of interest. In other words, strand invasion may occur within close proximity of the binding site of the targeting oligonucleotide.

In some embodiments, the method proceeds via different steps based on changes in temperature during the method. In some embodiments, the temperature used for initiating strand invasion is different from the optimum temperature for fragmenting by the transposase. In some embodiments, the temperature used for initiating strand invasion is below the optimum temperature for fragmenting by the transposase. In some embodiments, initiating strand invasion at a lower temperature promotes proper targeting of the transposome complexes based on the targeting oligonucleotide coated with a recombinase before fragmenting is initiated by an increase in temperature. These temperature changes can help to promote binding of the targeted transposome complexes to the sequence of interest in the target nucleic acid before fragmenting.

In some embodiments, initiating strand invasion is performed at 27° C. to 47° C. In some embodiments, initiating strand invasion is performed at 32° C. to 42° C. In some embodiments, initiating strand invasion is performed at 37° C.

In some embodiments, the fragmenting is performed at 45° C. to 65° C. In some embodiments, the fragmenting is performed at 50° C. to 60° C. In some embodiments, the fragmenting is performed at 55° C.

In some embodiments, initiating strand invasion is performed while the reaction solution lacks a component for transposase activity. For example, in some embodiments, a cofactor for the transposase is added to the transposome complexes after initiating invasion and before fragmenting. In some embodiments, the cofactor is Mg⁺⁺. In some embodiments, the Mg⁺⁺ concentration is 10 mM to 18 mM.

Methods using a targeted transposome complex comprising a targeting oligonucleotide coated in a recombinase can increase the probability of fragmenting occurring in close proximity to where the targeting oligonucleotide has bound the target nucleic acid. In some embodiments, the fragmenting occurs within 40, 30, 20, 15, 10, or 5 bases of the one or more sequences of interest in a nucleic acid sequence bound by the targeting oligonucleotide.

B. Methods Using Hybridizing of Targeting Oligonucleotides to Single-Stranded Nucleic Acid

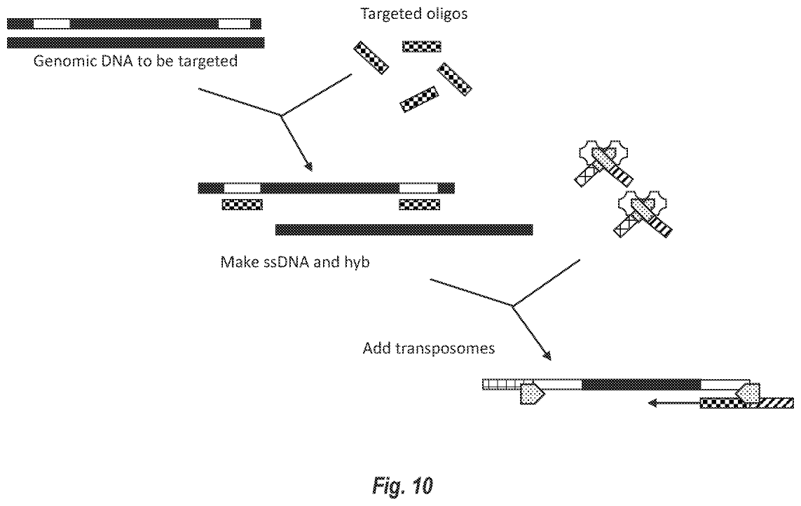

Transposases can mediate transposition and fragmentation of double-stranded nucleic acids. Therefore, selective generation of regions of double-stranded nucleic acid via binding of a targeting oligonucleotide to a single-stranded nucleic acid, such as single-stranded DNA, can be used in methods to generate tagged fragments. An exemplary method using targeting oligonucleotides is shown in FIG. 10.

A method of targeted generation of 5′ tagged fragments of nucleic acid may comprise hybridizing one or more targeting oligonucleotides to a sample comprising single-stranded nucleic acid. In some embodiments, a double-stranded target nucleic acid may be denatured to generate single-stranded nucleic acid. In some embodiments, double-stranded DNA is denatured to generate the single-stranded DNA. In some embodiments, denaturing is performed via an increase in temperature. In some embodiments, a double-stranded nucleic acid is denatured by increasing temperature to above the melting temperature (Tm) of the nucleic acid. In some embodiments, a sample comprising double-stranded DNA is heated to a temperature above 70° C. to promote denaturing of the double-stranded DNA into single-stranded DNA. In some embodiments, double-stranded nucleic acid is treated with urea and/or a pH change to generate single-stranded DNA.

In some embodiments, hybridizing one or more targeting oligonucleotides to a sample comprising single-stranded nucleic acid is performed by decreasing the temperature of a sample comprising single-stranded nucleic to allow binding of the one or more targeting oligonucleotides to the single-stranded nucleic acid.

In some embodiments, the one or more targeting oligonucleotides can each bind to a sequence of interest in the nucleic acid. In some embodiments, a targeting oligonucleotide is fully or partially complementary to a sequence of interest in the nucleic acid.

In some embodiments, hybridizing of the one or more targeting oligonucleotides to a single-stranded nucleic acid generates regions of double-stranded nucleic acid. While a transposase would not bind to regions of single-stranded nucleic acid, a transposase can bind to the double-stranded regions generating by hybridizing of the targeting oligonucleotides to the single-stranded nucleic acid. In some embodiments, hybridizing a targeting oligonucleotide to a sample comprising single-stranded nucleic acid generates a region of double-stranded nucleic acid that can be fragmented.

In some embodiments, a method comprises applying a transposome complex after hybridizing the one or more targeting oligonucleotides to the sample. In some embodiments, the transposome complex comprises a transposase; a first transposon comprising a 3′ transposon end sequence and a 5′ adaptor sequence; and a second transposon comprising a 5′ transposon end sequence, wherein the 5′ transposon end sequence is complementary to the 3′ transposon end sequence. In some embodiment, the method then comprises fragmenting the nucleic acid into a plurality of fragments by the transposase, by joining the 3′ end of the first transposon to the 5′ ends of the fragments to produce a plurality of 5′ tagged fragments.

In some embodiments, two or more targeting oligonucleotides with different sequences are hybridized. In some embodiments, methods with two or more targeting oligonucleotides can mediate fragmentation at two or more sites in the target nucleic acid. For example, the two or more targeting oligonucleotides may bind at the ends of a region of interest in the target nucleic acid, such that the fragmenting generates fragments comprising the region of interest. In other words, a method with two or more targeting oligonucleotides can generate a targeted library.

In some embodiments, multiple copies of a single targeting oligonucleotide are hybridized.

In some embodiments, only one type of targeting oligonucleotide is hybridized. In this way, the target nucleic acid is fragmented in a specific region. In some embodiments, the single targeting oligonucleotide is long enough to allow binding of two transposome complexes to the double-stranded nucleic acid generated by hybridizing the single targeting oligonucleotide to the sample comprising single-stranded nucleic acid. In some embodiments, the single targeting oligonucleotide comprises 80, 90, 100, 110, 120, 130, 140, 150, 160, 170, 180, 190, or 200 base pairs.

In some embodiments, the fragmenting occurs within the one or more sequences of interest in a nucleic acid sequence bound by the one or more targeting oligonucleotide.

C. Methods Using ShCAST

In some implementations, ShCAST (Scytonema hofmanni CRISPR associated transposase) targeted library preparation and enrichment may be used, as summarized in FIGS. 16A and 16B.

Targeted sequencing of specific genes using a separate enrichment step after library preparation may be time-consuming. For example, such a separate enrichment step may involve hybridizing oligonucleotide probes to library DNA and isolating the hybridized DNA on streptavidin-coated beads. Despite significant improvements in efficiency and time required, such separate enrichment protocols may take about two hours and many reagents which can made such protocols challenging to automate.

In comparison, methods using ShCAST as described herein can be used to prepare and enrich libraries for targeted sequencing of specific genes, using a single step for both preparation and enrichment.

In some embodiments, the first and/or second targeted transposome complex comprise a targeted transposome complex comprising ShCAST.

In some embodiments, at least one of the gRNA and the transposase are biotinylated, the composition further comprising a streptavidin-coated bead to which the at least one of the gRNA and transposase that is biotinylated is coupled. In this way, tagged fragments generated using a targeted transposome complex comprising ShCAST can be immobilized on streptavidin-coated beads.

In some embodiments, some or all steps of a method are performed in a reaction fluid that limits or inhibits non-specific binding of the nucleic acid by the transposase comprised in the ShCAST. In some embodiments, limiting or inhibiting non-specific binding of the transposase comprised in the ShCAST reduces off-target transposition reactions mediated by the transposase comprised in ShCAST. Such off-target transposition could occur if a transposase comprised in a ShCAST randomly binds a nucleic itself, instead the ShCAST being targeted to a sequence of interest by a gRNA bound to a sequence of interest. When off-target cleavage is reduced, most fragments will be generated from cleavage mediated by a targeted transposome complex. In this way, most tagged fragments will be prepared from one or more loci of interest (comprising one of more sequence of interest that can bind to one or more gRNA). Further, if a tagged fragment is prepared from two targeted transposome complexes, it will likely be of a size that can be sequenced and/or amplified. In contrast, when one or both transposome complex used to prepare a fragment are not properly targeted (for example, if the transposase comprised in the ShCAST binds directly to a nucleic acid without targeting by the gRNA), the fragment will likely be too large for amplifying and/or sequencing.

In some embodiments, the method is performed in a fluid having a condition for limiting binding of the complex directly by the transposase. In some embodiments, the condition for limiting binding of the complex directly by the transposase is a magnesium concentration of 15 mM or lower and/or with a concentration of Cas12K and/or transposase of 50 nM or lower.

In some embodiments, different steps of a method are performed under different conditions. In some embodiments, binding of the complex is performed under conditions that inhibit binding of the transposase to the double-stranded nucleic acid. In this way, non-targeted binding of ShCAST to a nucleic acid directly by the transposase is limited, and most ShCAST would be bound to a nucleic acid based on Cas12K association with gRNA targeted to one or more sequence of interest in a nucleic acid.

In some embodiments, after binding, conditions may be modified to promote cleavage by the transposase comprised in ShCAST. In some embodiments, a method comprises binding the complex to a double-stranded nucleic acid under conditions that inhibit binding of the double-stranded nucleic acid by the transposase comprised in the complex; and after the binding, promoting cleavage of the double-stranded nucleic acid by the complex.

In some embodiments, a transposase is absent or at low concentrations during the binding, and promoting cleavage comprises adding a transposase.

In some embodiments, an activatable transposase is comprised in the ShCAST. As used herein, an “activatable transposase” is one that is reversibly deactivated and that can be activated at a later time. For example, a reversibly deactivated transposase may lack a component for proper cleavage of nucleic acid, and this component may be added during a later step in a method.

In some embodiments, a transposase is reversibly deactivated during the binding and promoting cleavage comprises activating the transposase.

In some embodiments, the transposase is reversibly deactivated due to lack of one or more transposon, and activating the transposase comprises providing one or more transposons.

In some embodiments, the transposases add the amplification adapters to locations in the double-stranded nucleic acid. As used herein, an “amplification adapter” is any sequence useful for amplification (such as a binding site for an amplification primer). In this way, tagged fragments generated can be amplified without need for incorporating an additional amplification adapter. In some embodiments, an amplification adapter may be added to fragments (such as with ligation of the amplification adapter) after preparing tagged fragments.

D. Methods Comprising Pairing of Binding Partners

When a first paired binding partner is bound to a catalytically inactive endonuclease or zinc finger DNA-binding domain and a second binding partner is bound to the transposase, high resolution sequencing libraries can be generated.

Methods comprising pairing of binding partners may be analogous to CUT&Tag methods (See Kaya-Okur et al., Nature Communications 10:1930 (2019)). In such methods, a catalytically inactive endonuclease or zinc finger DNA-binding domain comprising a first binding partner is bound to a target nucleic acid. In some embodiments, the reaction is washed after this binding. Then, a transposase comprising a second binding partner is added. The transposase will localize to the catalytically inactive endonuclease or zinc finger DNA-binding domain based on affinity of the second binding partner for the first binding partner. These methods allow binding of the transposase to sites that have already been bound by the catalytically inactive endonuclease or zinc finger DNA-binding domain.

In some embodiments, methods are performed under conditions to limit binding of a catalytically inactive endonuclease or zinc finger DNA-binding domain. These conditions can limit off-target transposase binding. In some embodiments, low concentrations of magnesium or low concentration of a catalytically inactive endonuclease or zinc finger DNA-binding are used to reduce off-target transposase binding. In some embodiments, the likelihood of generating amplifiable PCR products from off-target binding is reduced. In some embodiments, limited off-target transposase binding means that random (i.e., non-targeted) transposase binding occurs with a low frequency and generally leads to fragments that are too large to be amplified and/or sequenced. In contrast, the use of targeted transposome complexes can be designed to prepare fragments of appropriate size for amplifying and/or sequencing.

As used herein, the first binding partner and the second binding partner may be referred to as “tags.” In some embodiments, a first tag is coupled to a first Cas-gRNA ribonucleoprotein (RNP, which comprises the Cas and that gRNA) and a second tag is coupled to a second Cas-gRNA RNP. In some examples, the method includes coupling the first tag to a first tag partner coupled to a substrate and coupling the second tag to a second tag partner coupled to the substrate. In some examples, the coupling is performed after the first and second Cas-gRNA RNPs respectively are hybridized to the first and second subsequences. In some examples, the first and amplification adapters are added after the first and second tags respectively are added to the first and second tag partners.

In some examples, the first and second tags include biotin. In some examples, the first and second tag partners include streptavidin. In some examples, the substrate includes a bead. In some examples, the Cas-gRNA RNP comprises Cas12k. In some examples, the transposase comprises Tn5 or a Tn7 like transposase.

In some embodiments, combining a sample comprising a double-stranded nucleic acid with one or more transposome complex that is targeted comprises combining the sample with a zinc finger DNA-binding domain or a catalytically inactive endonuclease, wherein the zinc finger DNA-binding domain or catalytically inactive endonuclease is bound to a first binding partner, and adding the transposase and first and second transposons, wherein the transposase is bound to a second binding partner, wherein the transposase can bind to the zinc finger DNA-binding domain or catalytically inactive endonuclease by pairing of the first and second binding partners.

In some embodiments, the method comprises washing after the combining and before the adding. In some embodiments, cell-free DNA is not treated with a protease before combining with the zinc finger DNA-binding domain.

E. Methods of Generating Targeted Fragments with Two Targeted Transposome Complexes

In some embodiments, polynucleotides (such as a target nucleic acid) may be cut at any suitable pairs of locations to form fragments. After forming fragments using methods disclosed herein, any suitable amplification primers may be coupled to the resulting ends of the fragments. The fragments then may be amplified and sequenced.

In methods with a first and second transposome complex that are both targeted, the complexes may be designed to produce specific desired fragments. In some embodiments, methods with a first and second transposome complex that are both targeted can generate targeted or enriched libraries. These targeted or enriched libraries may comprise a higher percentage of library fragments comprising an enrichment target region. This enrichment target region could be, for example, a gene of interest for sequencing.

In some embodiments, the first transposome complex that is targeted and the second transposon complex that is targeted bind to opposite strands of the double-stranded nucleic acid, wherein the first transposome complex binds to a first transposome complex binding site and wherein the second transposome complex binds to a second transposome complex binding site. In some embodiments, the first 5′ tagged target fragments and the second 5′ tagged target fragments comprise nucleic acid sequences comprised in in a region of the double-stranded nucleic acid between the first transposome complex binding site and the second transposome complex binding site. In some embodiments, the first 5′ tagged target fragments and the second 5′ tagged fragments are at least partially complementary.

In some embodiments, the catalytically inactive endonuclease or zinc finger DNA-binding domain comprised in the first transposome complex that is a targeted transposome complex and the second transposome complex that is a targeted transposome complex are different. A representative method using two targeted transposome complexes comprising catalytically inactive endonucleases is shown in FIG. 11.

In some embodiments, the catalytically inactive endonuclease or zinc finger DNA-binding domain of the first transposome complex that is a targeted transposome complex and the second transposome complex that is a targeted transposome complex bind to different sequences of interest in a given region of interest in a target nucleic acid.

F. Samples and Target Nucleic Acids

In some embodiments, a sample comprises target nucleic acid. In some embodiments, the sample comprises DNA. In some embodiments, the DNA is genomic DNA. In some embodiments, the target nucleic acid is double-stranded DNA.

In some embodiments, the target nucleic acid is single-stranded DNA. While single-stranded DNA cannot be fragmented by transposases, method described herein describe means to generate regions of double-stranded DNA, such as by hybridizing targeting oligonucleotides to single-stranded DNA.

The biological sample can be any type that comprises nucleic acid. For example, the sample can comprise nucleic acid in a variety of states of purification, including purified nucleic acid. However, the sample need not be completely purified, and can comprise, for example, nucleic acid mixed with protein, other nucleic acid species, other cellular components, and/or any other contaminant. In some embodiments, the biological sample comprises a mixture of nucleic acid, protein, other nucleic acid species, other cellular components, and/or any other contaminant present in approximately the same proportion as found in vivo. For example, in some embodiments, the components are found in the same proportion as found in an intact cell. In some embodiments, the biological sample has a 260/280 absorbance ratio of less than or equal to 2.0, 1.9, 1.8, 1.7, 1.6, 1.5, 1.4, 1.3, 1.2, 1.1, 1.0, 0.9, 0.8, 0.7, or 0.60. In some embodiments, the biological sample has a 260/280 absorbance ratio of at least 2.0, 1.9, 1.8, 1.7, 1.6, 1.5, 1.4, 1.3, 1.2, 1.1, 1.0, 0.9, 0.8, 0.7, or 0.60. Because the methods provided herein allow nucleic acid to be bound to solid supports, other contaminants can be removed merely by washing the solid support after surface bound tagmentation occurs. The biological sample can comprise, for example, a crude cell lysate or whole cells. For example, a crude cell lysate that is applied to a solid support in a method set forth herein, need not have been subjected to one or more of the separation steps that are traditionally used to isolate nucleic acids from other cellular components. Exemplary separation steps are set forth in Maniatis et al., Molecular Cloning: A Laboratory Manual, 2d Edition, 1989, and Short Protocols in Molecular Biology, ed. Ausubel, et al, hereby incorporated by reference.

In some embodiments, the sample that is applied to the solid support has a 260/280 absorbance ratio that is less than or equal to 1.7.

Thus, in some embodiments, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising nucleic acid.

In some embodiments, the sample is blood. In some embodiments, the sample is a cell lysate. In some embodiments, the cell lysate is a crude cell lysate. In some embodiments, the method further comprises lysing cells in the sample after applying the sample to a solid support to generate a cell lysate.

In some embodiments, the sample is a biopsy sample. In some embodiments, the biopsy sample is a liquid or solid sample. In some embodiments, a biopsy sample from a cancer patient is used to evaluate sequences of interest to determine if the subject has certain mutations or variants in predictive genes.

One advantage of the methods and compositions presented herein that a biological sample can be added to a flow cell and subsequent lysis and purification steps can all occur in the flow cell without further transfer or handling steps, simply by flowing the necessary reagents into the flow cell.

In some embodiments, a protective element may be incorporated into a polynucleotide (such as a target nucleic acid or a double-stranded fragment generated by tagmentation). For example, the protective element may be added to a target nucleic acid before tagmentation or a double-stranded nucleic acid fragment after tagmentation in any of the methods described herein. As used herein, the term “protective element,” when used in reference to the 5′ or 3′ end of a polynucleotide, is intended to mean an element that inhibits modification of that end of the polynucleotide. Illustratively, the protective element may inhibit action of one or more enzymes upon that end of the polynucleotide, such as action of a 5′ or 3′ exonuclease. Non-limiting examples of protective elements include a hairpin sequence that is ligated to the 5′ and 3′ strands of the end of a double-stranded polynucleotide, a modified base (e.g., including a phosphorothioate bond or 3′ phosphate), or a dephosphorylated base.

G. Gap-Fill Ligation

In some embodiments, gaps in the DNA sequence left after the transposition event can also be filled in using a strand displacement extension reaction, such one comprising a Bst DNA polymerase and dNTP mix. In some embodiments, a gap-fill ligation is performed using an extension-ligation mix buffer.

In some embodiments, a method comprises treating the plurality of 5′ tagged fragments with a polymerase and a ligase to extend and ligate the strands to produce fully double-stranded tagged fragments.

The library of double-stranded DNA fragments can then optionally be amplified (such as with cluster amplification) and sequenced with a sequencing primer.

H. Amplification

The present disclosure further relates to amplification of tagged fragments produced according to the methods provided herein. In some embodiments, immobilized tagged fragments are amplified on a solid support. In some embodiments, the solid support is the same solid support upon which the surface bound tagmentation occurs. In such embodiments, the methods and compositions provided herein allow sample preparation to proceed on the same solid support from the initial sample introduction step through amplification and optionally through a sequencing step.

For example, in some embodiments, immobilized tagged fragments are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays”. The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from immobilized DNA fragments produced according to the methods provided herein. For example, one or more clusters or colonies can be formed via solid-phase PCR whether one or both primers of each pair of amplification primers are immobilized.

In other embodiments, tagged fragments are amplified in solution. For example, in some embodiments, tagged fragments are cleaved or otherwise liberated from the solid support and amplification primers are then hybridized in solution to the liberated molecules. In other embodiments, amplification primers are hybridized to tagged fragments for one or more initial amplification steps, followed by subsequent amplification steps in solution. In some embodiments, an immobilized nucleic acid template can be used to produce solution-phase amplicons.

It will be appreciated that any of the amplification methodologies described herein or generally known in the art can be utilized with universal or target-specific primers to amplify tagged fragments. Suitable methods for amplification include, but are not limited to, the polymerase chain reaction (PCR), strand displacement amplification (SDA), transcription mediated amplification (TMA) and nucleic acid sequence based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify immobilized DNA fragments. In some embodiments, primers directed specifically to the nucleic acid of interest are included in the amplification reaction.

Other suitable methods for amplification of nucleic acids can include oligonucleotide extension and ligation, rolling circle amplification (RCA) (Lizardi et al., Nat. Genet. 19:225-232 (1998), which is incorporated herein by reference) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference) technologies. It will be appreciated that these amplification methodologies can be designed to amplify immobilized DNA fragments. For example, in some embodiments, the amplification method can include ligation probe amplification or oligonucleotide ligation assay (OLA) reactions that contain primers directed specifically to the nucleic acid of interest. In some embodiments, the amplification method can include a primer extension-ligation reaction that contains primers directed specifically to the nucleic acid of interest. As a non-limiting example of primer extension and ligation primers that can be specifically designed to amplify a nucleic acid of interest, the amplification can include primers used for the GoldenGate assay (Illumina, Inc., San Diego, Calif.) as exemplified by U.S. Pat. Nos. 7,582,420 and 7,611,869, each of which is incorporated herein by reference in its entirety.

Exemplary isothermal amplification methods that can be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587, each of which is incorporated herein by reference in its entirety. Other non-PCR-based methods that can be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyperbranched strand displacement amplification which is described in, for example Lage et al., Genome Research 13:294-307 (2003), each of which is incorporated herein by reference in its entirety. Isothermal amplification methods can be used with the strand-displacing Phi 29 polymerase or Bst DNA polymerase large fragment, 5′→3′ exo—for random primer amplification of genomic DNA. The use of these polymerases takes advantage of their high processivity and strand displacing activity. High processivity allows the polymerases to produce fragments that are 10-20 kb in length. As set forth above, smaller fragments can be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another nucleic acid amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993), incorporated herein by reference in its entirety. The first rounds of amplification are carried out to allow a multitude of initiations on heat denatured DNA based on individual hybridization from the randomly synthesized 3′ region. Due to the nature of the 3′ region, the sites of initiation are contemplated to be random throughout the genome. Thereafter, the unbound primers can be removed, and further replication can take place using primers complementary to the constant 5′ region.

I. Sequencing and Resequencing

Initial sequencing (and potential resequencing) can be performed using a variety of different methods.

The present disclosure further relates to sequencing of tagged fragments produced according to the methods provided herein. In some embodiments, a method comprises sequencing one or more of the 5′ tagged fragments or fully double-stranded tagged fragments.

The tagged fragments produced by transposome-mediated tagmentation can be sequenced according to any suitable sequencing methodology, such as direct sequencing, including sequencing by synthesis, sequencing by ligation, sequencing by hybridization, nanopore sequencing and the like. In some embodiments, the tagged fragments are sequenced on a solid support. In some embodiments, the solid support for sequencing is the same solid support upon which surface-bound tagmentation occurs. In some embodiments, the solid support for sequencing is the same solid support upon which the amplification occurs.

One exemplary sequencing methodology is sequencing-by-synthesis (SBS). In SBS, extension of a nucleic acid primer along a nucleic acid template (e.g. a target nucleic acid or amplicon thereof) is monitored to determine the sequence of nucleotides in the template. The underlying chemical process can be polymerization (e.g. as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template.

Flow cells provide a convenient solid support for housing amplified DNA fragments produced by the methods of the present disclosure. One or more amplified DNA fragments in such a format can be subjected to an SBS or other detection technique that involves repeated delivery of reagents in cycles. For example, to initiate a first SBS cycle, one or more labeled nucleotides, DNA polymerase, etc., can be flowed into/through a flow cell that houses one or more amplified nucleic acid molecules. Those sites where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be adapted for application of pyrosequencing to amplicons produced according to the present disclosure are described, for example, in WIPO Pat. App. Pub. No. WO 2012058096, US 2005/0191698 A1, U.S. Pat. Nos. 7,595,883, and 7,244,559, each of which is incorporated herein by reference.

Some embodiments can utilize methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, for example, Deamer et al. Trends Biotechnol. 18, 147-151 (2000); Deamer et al. Acc. Chem. Res. 35:817-825 (2002); Li et al. Nat. Mater. 2:611-615 (2003), the disclosures of which are incorporated herein by reference). In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008), the disclosures of which are incorporated herein by reference).

Exemplary methods for array-based expression and genotyping analysis that can be applied to detection according to the present disclosure are described in U.S. Pat. Nos. 7,582,420; 6,890,741; 6,913,884 or 6,355,431 or US Pat. Pub. Nos. 2005/0053980 A1; 2009/0186349 A1 or US 2005/0181440 A1, each of which is incorporated herein by reference.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for detection of target nucleic acids. Exemplary flow cells are described, for example, in US 2010/0111768 A1 and US Pub. No. 2012/0270305 A1, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in US Pub. No. 2012/0270305, which is incorporated herein by reference.

J. Preserving Contiguity Information when Sequencing a Target Nucleic Acid

In some embodiments, contiguity information is preserved based on a targeting oligonucleotide.

In some embodiments, a method of preserving contiguity information when sequencing a target nucleic acid comprises producing tagged fragments of the target nucleic acid with method comprising a targeted transposome complex comprising a targeting oligonucleotide coated with a recombinase; sequencing the 5′ tagged fragments or fully double-stranded tagged fragments to provide sequences of the fragments; grouping sequences of fragments that comprise the sequence of the same targeting oligonucleotide; and determining that a group of sequences were in proximity within the target nucleic acid if they comprise the sequence of the same targeting oligonucleotide.

Contiguity information may also be preserved based on adaptor sequences that comprise unique molecular identifiers (UMI) sequences. In some embodiments, a method of preserving contiguity information when sequencing a target nucleic acid comprises producing tagged fragments of the target nucleic acid using a targeted transposome complex comprising a targeting oligonucleotide coated with a recombinase, wherein one or more adapter sequence comprises a unique molecular identifier (UMI) associated with a single targeting oligonucleotide sequence; sequencing the 5′ tagged fragments or fully double-stranded tagged fragments to provide sequences of the fragments; grouping sequences of fragments that comprise the sequence of the same UMI; and determining that a group of sequences were in proximity within the target nucleic acid if they comprise the sequence of the same UMI.

Targeted transposomes may also be used in methods of generating a physical map of immobilized polynucleotides. The methods can advantageously be exploited to identify clusters likely to contain linked sequences (i.e., the first and second portions from the same target polynucleotide molecule). The relative proximity of any two clusters resulting from an immobilized polynucleotide thus provides information useful for alignment of sequence information obtained from the two clusters. Specifically, the distance between any two given clusters on a solid surface is positively correlated with the probability that the two clusters are from the same target polynucleotide molecule, as described in greater detail in WO 2012/025250, which is incorporated herein by reference in its entirety.

As an example, in some embodiments, long DNA molecules stretching out over the surface of a flow cell are tagmented in situ, resulting in a line of connected DNA bridges across the surface of the flow cell. Further, a physical map of the immobilized DNA. The physical map thus correlates the physical relationship of clusters after immobilized DNA is amplified. Specifically, the physical map is used to calculate the probability that sequence data obtained from any two clusters are linked, as described in the incorporated materials of WO 2012/025250.

In some embodiments, the physical map is generated by imaging the DNA to establish the location of the immobilized DNA molecules across a solid surface. In some embodiments, the immobilized DNA is imaged by adding an imaging agent to the solid support and detecting a signal from the imaging agent. In some embodiments, the imaging agent is a detectable label. Suitable detectable labels include, but are not limited to, protons, haptens, radionuclides, enzymes, fluorescent labels, chemiluminescent labels, and/or chromogenic agents. For example, in some embodiments, the imaging agent is an intercalating dye or non-intercalating DNA binding agent. Any suitable intercalating dye or non-intercalating DNA binding agent as are known in the art can be used, including, but not limited to those set forth in U.S. 2012/0282617, which is incorporated herein by reference in its entirety.

In some embodiments, the immobilized DNA duplexes are further fragmented to liberate a free end prior to strand exchange and cluster generation. Cleaving bridged structures can be performed using any suitable methodology as is known in the art, as exemplified by the incorporated materials of WO 2012/025250. For example, cleavage can occur by incorporation of a modified nucleotide, such as uracil as described in WO 2012/025250, by incorporation of a restriction endonuclease site, or by applying solution-phase transposome complexes to the bridged DNA structures, as described elsewhere herein.

In certain embodiments, a plurality of nucleic acid is flowed onto a flow cell comprising a plurality of nano-channels, the nano-channel having a plurality of transposome complexes immobilized thereto. As used herein, the term nano-channel refers to a narrow channel into which a long linear nucleic acid molecule is flowed. In some embodiments, no more than 1, 2, 3, 4, 5, 6, 7, 8, 9, 10, 11, 12, 13, 14, 15, 16, 17, 18, 19, 20, 30, 40, 50, 6070, 80, 90, 100, 200, 300, 400, 500, 600, 700, 800, 900 or no more than 1000 individual long strands of target DNA are flowed into each nano-channel. In some embodiments the individual nano-channels are separated by a physical barrier which prevents individual long strands of target DNA from interacting with multiple nano-channels. In some embodiments, the solid support comprises at least 10, 50, 100, 200, 500, 1000, 3000, 5000, 10000, 30000, 50000, 80000 or 100000 nano-channels. In some embodiments, transposomes bound to the surface of a nano-channel tagment the DNA. Contiguity mapping can then be performed, for example, by following the clusters down the length of one of these channels. In some embodiments, the long strand of target DNA can be at least 0.1 kb, 1 kb, 2 kb, 3 kb, 4 kb, 5 kb, 6 kb, 7 kb, 8 kb, 9 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, 1000 kb, 5000 kb, 10000 kb, 20000 kb, 30000 kb, or 50000 kb in length. In some embodiments, the long strand of target DNA is no more than 0.1 kb, 1 kb, 2 kb, 3 kb, 4 kb, 5 kb, 6 kb, 7 kb, 8 kb, 9 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 150 kb, 200 kb, 250 kb, 300 kb, 350 kb, 400 kb, 450 kb, 500 kb, 550 kb, 600 kb, 650 kb, 700 kb, 750 kb, 800 kb, 850 kb, 900 kb, 950 kb, or no more than 1000 kb in length. As an example, a flow cell having 1000 or more nano-channels with mapped immobilized tagmentation products in the nano-channels can be used to sequence the genome of an organism with short ‘positioned’ reads. In some embodiments, mapped immobilized tagmentation products in the nano-channels can be used resolve haplotypes. In some embodiments, mapped immobilized tagmentation products in the nano-channels can be used to resolve phasing issues.

IV. Methods Using Targeted Transposomes Complex with Samples Comprising Cell-Free DNA

Targeted transposomes described herein may be used for targeted transposition within a simplified library preparation and enrichment protocol. In some embodiments, the simplified protocol requires less time or user steps compared to existing protocols. In some embodiments, the one or more nucleic acid sequences of interest are comprised in DNA associated with histones. In some embodiments, the DNA associated with histones is cell-free DNA.

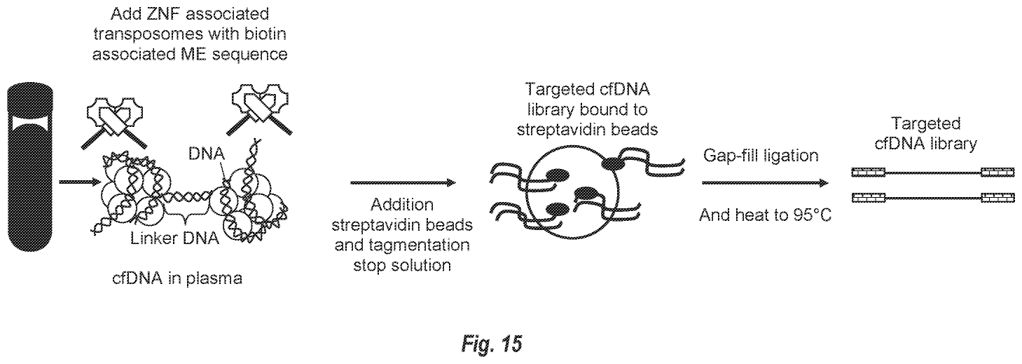

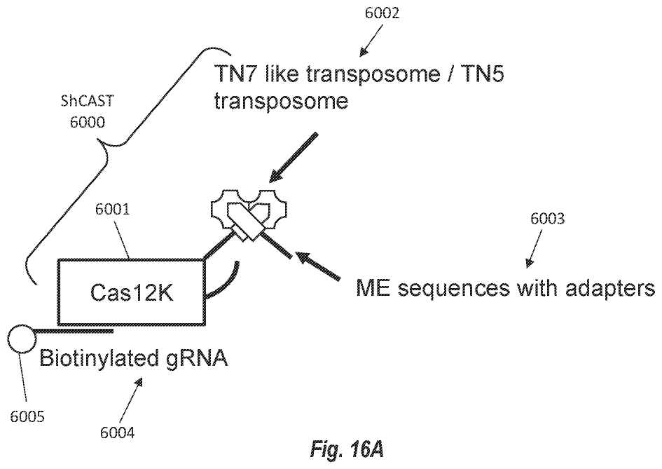

In some embodiments, the simplified library preparation and enrichment protocol is for use with cell-free DNA (cfDNA), such as the exemplary method shown in FIG. 15. Present library preparation for cfDNA commonly involves several steps: cfDNA extraction from plasma (30 minutes), end repair (30 minutes), A-tailing (30 minutes), ligation of non-random unique molecular identifiers (UMIs) (30 minutes), ligation of adaptors (30 minutes), and SPRI clean-up followed by PCR amplification (˜30 minutes). The cfDNA extraction from plasma in standard methods may include a protease step (for example Proteinase K, as described in Illumina Document #1000000001856 v06 (April 2020) providing the protocol for VeriSeq NIPT). Based on these steps, cfDNA library preparation is a time consuming and inefficient process that is challenging to automate.

Cell-free DNA (cfDNA) in plasma is known to exist in association with histones (See Marshman et al., Cell Death and Disease (2016) 7, e2518 and Rumore and Steinman J. Cin Inv. 86:69-74 (1990)). A key challenge in performing tagmentation directly in plasma samples is the removal of histones from cfDNA. Methods to remove histones may involve a protease step, wherein this protease can also degrade proteins involved in tagmentation. For example, the cfDNA extraction from plasma in the VeriSeq Non-Invasive Prenatal Testing (NIPT) method (Illumina) includes a protease step (Proteinase K, as described in the VeriSeq NIPT Solution Package Insert, Illumina Document #1000000001856 v06 (April 2020)) followed by multiple wash steps before library preparation. Targeting of transposomes to specific sequences of interest (such as genes within a genome), without requiring removal of histones, could significantly simplify workflows with samples comprising cfDNA.

Zinc finger DNA-binding domains can target zinc finger nucleases to specific regions of the genome for editing (See Costa et al., Genome Editing Using Engineered Nucleases and Their Use in Genomic Screening, PMID: 29165977, in Assay Guidance Manual (Markossian et al., editors) (2017)) Specifically, ZFNs retain the ability to efficiently cut DNA bound to histones, while Cas9 nucleases are strongly inhibited when DNA is bound to a histone (See Yarringon et al., PNAS 115(38):9351-9358 (2018)).

In some embodiments, DNA bound to histones may be comprised in a nucleosome. As used herein, a “nucleosome” refers to a structure consisting of a segment of DNA wound around eight histone proteins. In some embodiments, the DNA bound to histones is cell-free DNA. Exemplary cell-free DNA may be cfDNA comprised in blood samples from pregnant women (wherein the cfDNA may be from the fetus) or patients with known or suspected cancer (wherein the cfDNA may be from tumor cells).

In some embodiments, targeted transposomes are targeted to one or more region in cfDNA by a zinc finger DNA-binding domain. In some embodiments, histone-bound DNA (such as cfDNA) is tagmented using targeted transposomes comprising a zinc finger DNA-binding domain.

In some embodiments, the method further comprises adding an affinity binding partner on a solid support after fragmenting, wherein the tagged target fragments are bound to the solid support. In some embodiments, the fragmenting is stopped before adding the affinity element on the solid support. In some embodiments, the fragmenting is stopped by addition of a solution comprising proteinase K and/or SDS.

For example, a transposome complex comprising a zinc finger DNA-binding domain can be targeted to specific sequences of interest within cfDNA, as shown in FIG. 15. In some embodiments, a zinc finger DNA-binding domain comprised in the targeted transposome may bind to a sequence comprised within or near an oncogene to generate a targeted library from the cfDNA within a sample from a patient with cancer to assess whether gain-of-function mutations are present in the cfDNA. Alternatively, a zinc finger DNA-binding domain comprised in the targeted transposome may bind to a sequence comprised within or near a tumor suppressor gene to generate a specific library from the cfDNA to assess whether loss-of-function mutations (i.e., activating mutations) are present in the cfDNA. In this way, such targeted transposons can be used to generate targeted libraries for assessing changes in cancer cells that are associated with more aggressive tumors or associated with a poorer prognosis.

Similarly, targeted libraries from cfDNA can be used to assess for specific gene sequences that are associated with genetic diseases. These genetic diseases may be known inheritable diseases caused by known changes in gene sequences such as Tay-Sachs disease, cystic fibrosis, and many more well-known to those in the field. In some embodiments, a zinc finger DNA-binding domain comprised in the targeted transposome may bind to a sequence comprised within or near genes associated with inheritable diseases to generate a targeted library. In some embodiments, a targeted library may be for sequencing areas of genes of interest for SNPs or other mutations in prenatal testing using maternal plasma comprising cfDNA from a fetus.

### V. Methods of Sorting and Selection of Single Cell Nucleic Acids

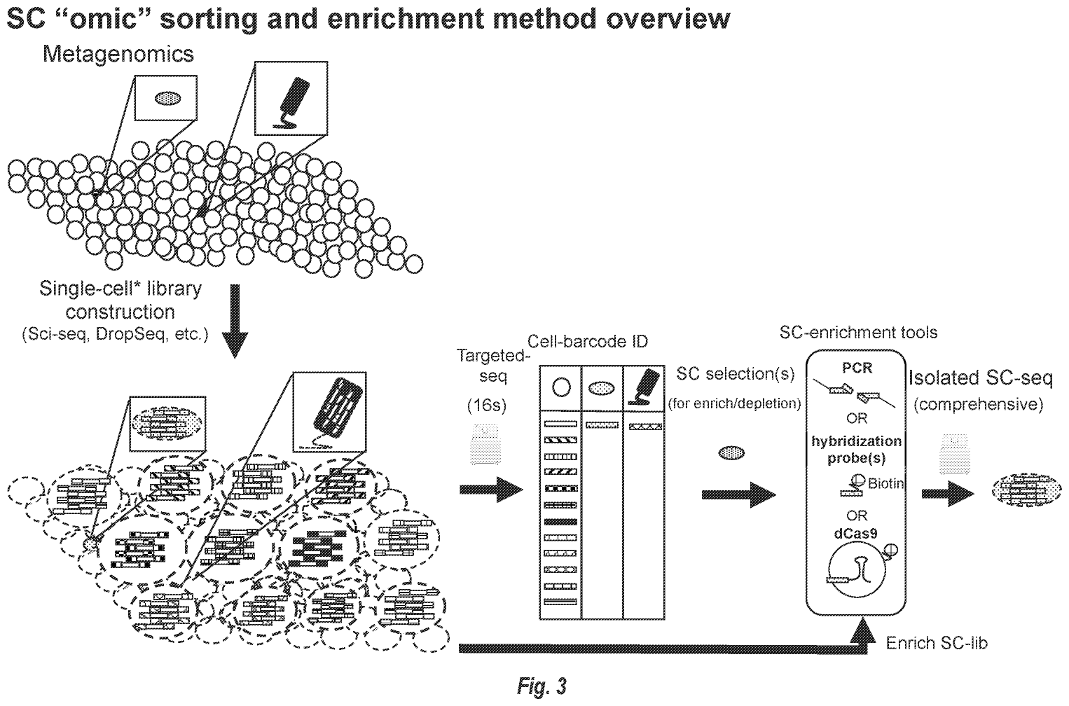

Described herein are methods utilizing sc-NGS (single-cell next generation sequencing) methods in combination with nucleic acid selection techniques to enable cellular sorting based on “-omic” feature(s). The methods may involve targeting unique cellular barcodes to enrich or deplete sc-library members. The present workflow comprising a two-sequencing step workflow provides a tractable methodology in which an initial sequencing run creates a cellular database used to decide which cells to obtain additional ‘omic’ data on in a second more comprehensive sequencing run after selection of desired cells. FIG. 3 provides an overview of such a method of sorting and selection, wherein initial 16s sequencing is used to determine cell-barcode ID's of interest, followed by enrichment of desired samples or depletion of unwanted samples. After enrichment/depletion, desired samples can undergo comprehensive sequencing.

In some embodiments, cell selection is achieved by depleting unwanted samples, such as abundant cells of low interest, from a sc-library based on their assigned UBCs. Secondary sequencing after this depletion can characterize DNA libraries generated from desired samples, i.e. cells of interest that may be rare within the library. In some embodiments, cell selection is achieved by enriching desired samples using their assigned UBCs from the sc-library. These desired samples may be rare or of low abundance in the sample.

### VI. Methods of Characterizing Desired Samples in a Mixed Pool of Samples

Described herein is a method of characterizing desired samples in a mixed pool of samples comprising both desired samples and unwanted samples. In some embodiments, the method comprises initially sequencing a library comprising a plurality of nucleic acid samples from the mixed pool of samples to produce sequencing data from double-stranded nucleic acid. In some embodiments, each nucleic acid library comprises nucleic acids from a single sample and a unique sample barcode to distinguish the nucleic acids from the single sample from the nucleic acids from other samples in the library.

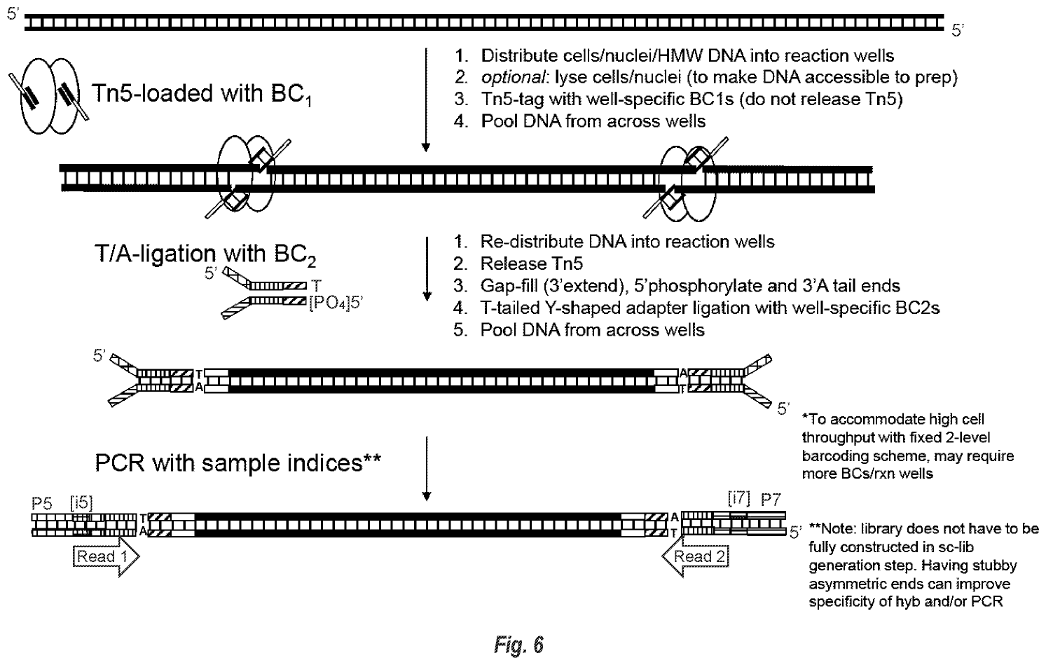

The present method can be a cost-effective means to characterize single cells within a given population, based on barcodes associated with cells having a desired genomic feature (where the desired genomic feature could be the presence of a specific gene mutation, the methylation status of a given gene, etc.). This desired genomic feature can be determined from an initial sequencing that is followed by a selection step and then resequencing to provide further information on the single cells of interest. Representative methods of incorporating barcodes are presented in FIGS. 5 and 6.

In some embodiments, the method also comprises analyzing the sequencing data and identifying unique sample barcodes associated with sequencing data from desired samples; performing a selection step on the library comprising enriching nucleic acid samples from desired samples and/or depleting nucleic acid samples from unwanted samples; and resequencing the nucleic acid library.

In some embodiments, the resequencing is an orthogonal resequencing. As used herein, “orthogonal resequencing” refers a resequencing that analyzes a different physiologic characteristic compared to the initial sequencing. For example, the initial sequencing may assess methylation status, and the resequencing may be a comprehensive genome wide sequencing of cells having a desired methylation pattern. In other words, the initial sequencing and the resequencing may assess the same characteristic of the mixed pool of samples, but the initial sequencing and the resequencing may also assess different characteristics of the desired samples.

An advantage of the present methods is that certain steps that may normally be used to generate sequencing data on a desired sample can be avoided. In other words, the present methods may be faster or easier that other methods or may avoid steps that could bias the results. In some embodiments, the method does not employ cell sorting-based enrichment methods. In some embodiments, the method does not employ FACS. In some embodiments, the method does not employ FACS based on cell size, morphology, or surface protein expression. In some embodiments, the method does not employ microfluidics. In some embodiments, the method does not employ whole genome amplification. Avoiding these steps in the present method may reduce the time and cost necessary for generating comprehensive sequencing data on desired samples. In addition, avoiding these steps may avoid bias that comes from certain methods (such as relying on surface protein expression to sort cells with FACS methodology).

Further, the present methods of sequencing and analysis can be performed using a sequencing system, without also requiring a FACS machine, etc.

In some embodiments, the initial sequencing results can be used to guide the selection step, without the initial sequencing being biased by a sorting step beforehand. With the present method, one skilled in the art can sort a plurality of single cell libraries by initial sequencing for a trait of interest and use those initial sequence result to determine which cells are the desired cells, and then select for the desired cells and resequence.

Other advantages of the present method will be described herein.

A. Preparation of Libraries

The initial sequencing step of these methods may be any means of generating a library comprising a plurality of nucleic acid samples from a mixed pool of samples. In some embodiment, the library is a single cell library (sc-library). As used herein, a “single cell library” or “sc-library” refers to a library generated from single cells within a mixed population of cells. However, the library may also be a library from a single nucleus, virus, or high molecular weight (HMW) DNA within a mixed population. Thus, the present method can be used with a variety of mixed populations, and any method described for use with a sc-library could be used for other types of libraries.

In some embodiments, the present methods are performed after indexing of libraries but before a comprehensive sequencing of libraries.

In some embodiments, a nucleic acid library comprises nucleic acids from a single sample comprising a unique sample barcode to distinguish the nucleic acids from the single sample from the nucleic acids from other samples in the library. A wide variety of means of generating such libraries are well-known in the art. An advantage of the present method is that it can be used with libraries that are generated via a number of different ways. As such, one skilled in the art could choose a specific method to generate a library comprising a plurality of nucleic acid samples from a mixed pool of samples based on their own preference and perform initial sequencing. Then, the disclosed methods could be used for selection based on unique sample barcodes, followed by resequencing.

Representative methods of sc-sequencing include those of WO 2016/130704, which are incorporated by reference herein. In some embodiments, the method comprises a step of spatially separating the nucleic acid samples before incorporating a unique sample barcode.

These methods are applicable to any sc-library generation and sequencing methods employing unique cellular barcodes (UBCs) or unique sample barcodes. Exemplary sc-library generation/sequencing methods include Biorad ddSEQ (for example, using the Illumina Bio-Rad SureCell WTA 3′ Library Prep Kit), various 10X Genomics systems (such as Chromium Single Cell Expression), Drop-Seq (See Macosko et al., Cell 161(5):1202-1214 (2015)), InDrop™ (1CellBio), Tapestri™ Platform (MissionBio), Split-Seq (See Rosenburg et al., Science 360(6385):176-182 (2018)), or Illlumina's Single Cell Combinatorial Indexing Sequencing (SCI-seq, See Cao et al., Science 357(6352): 661-667 (2017)), all of which are incorporated by reference for disclosure of library generation and sequencing methods.

In some embodiments, the method comprises tagmentation prior to sequencing a plurality of nucleic acid samples from the mixed pool of samples. In some embodiments, libraries are generated using tagmentation. In some embodiments, the tagmentation incorporates a unique sample barcode into each nucleic acid sample.

In some embodiments, universal primers are incorporated into each nucleic acid sample within a nucleic acid library. In some embodiments, the universal primers are incorporated into each nucleic acid sample during preparation of the libraries. In some embodiments, the universal primers are P5 and P7 primers. In some embodiments, P5 and P7 sequences are incorporated into each nucleic acid sample within a nucleic acid library.

In some embodiments, i5 and i7 sequences are incorporated into each nucleic acid sample within a nucleic acid library. In some embodiments, i5 and i7 sequences are incorporated into each nucleic acid sample during preparation of the libraries.

B. Initial Sequencing

In some embodiments, untargeted initial sequencing may be beneficial to characterize a plurality of single cells, after which selection and resequencing can be performed to further analyze single cells of interest with the population. In some embodiments, initial sequencing identifies unique sample barcodes associated with unwanted samples. In some embodiments, initial sequencing identifies unique sample barcodes associated with desired samples.

In some embodiments, targeted initial sequencing can determine cells of interest (i.e., determine the desired samples) within a population of single cells, and libraries generated from these cells of interest can then be selected and resequenced to provide additional information.

In some embodiments, the initial sequencing step comprises targeted sequencing and the resequencing step comprises whole genome sequencing. In some embodiments, initial sequencing may be gene-specific sequencing. In some embodiments, initial sequencing may be 16s sequencing.

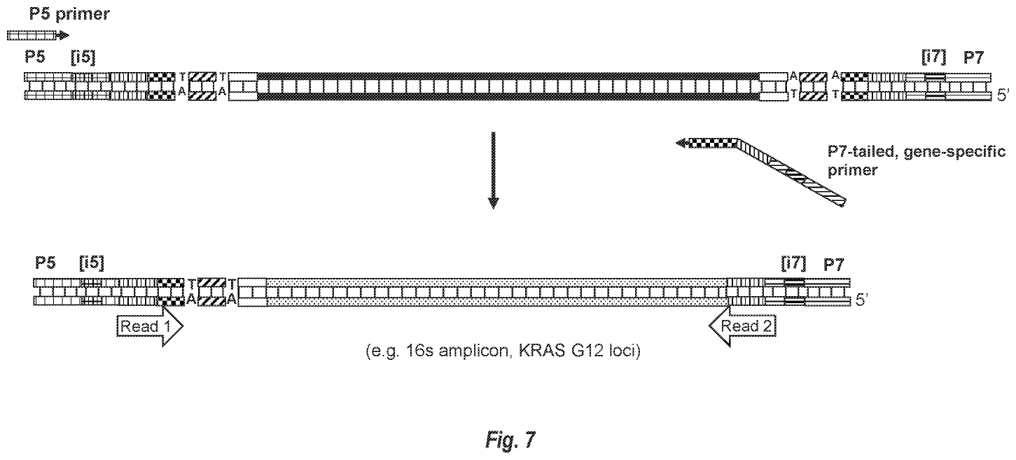

In some embodiments, the initial sequencing step comprises targeted sequencing with one or more gene-specific primers (as exemplified in FIG. 7). In some embodiments, the gene-specific primer comprises a universal primer tail.

In some embodiments, the initial sequencing step does not comprise whole genome sequencing and the resequencing step comprises whole genome sequencing. In other words, the initial sequencing may be less comprehensive, and the resequencing is more comprehensive. Such an approach could dramatically reduce the time/cost necessary to generate comprehensive data on desired samples by avoiding the resequencing of unwanted samples.

In some embodiments, the initial sequencing step comprises ribosomal sequencing and the resequencing step comprises whole genome sequencing. In some embodiments, ribosomal sequencing comprises 16s, 18s, or internal transcribed spacer sequencing. In some embodiments, the internal transcribed spacer region is located between the 16s and 23s rRNA genes. In some embodiments, ribosomal sequencing is used to determine species within a sample comprising a mixed pool of samples comprising samples from different species. For example, ribosomal sequencing may be used to determine bacterial species within a metagenomics sample. In some embodiments, resequencing comprises whole genome sequencing of the species of interest, after enriching these desired samples from species of interest or depleting unwanted samples from species not of interest.

In some embodiments, initial sequencing characterizes the cell population and then is followed by resequencing. For example, initial sequencing could identify cells of a desired cell type within a blood sample, and resequencing could focus specifically on these cells.
1. Targeted Initial Sequencing

In some embodiments, initial sequencing is targeted sequencing. As used herein, targeted sequencing refers to sequencing of region of a target nucleic acid. For example, targeted sequencing may be sequencing of a particular gene within a target genome.

FIG. 7 shows an example of how targeted initial sequencing may be performed. A sc-library comprising a plurality of cellular nucleic acid libraries can be prepared, with each library marked with one or more UBCs. Fragments in each cellular nucleic acid library comprise P5 sequences at one end and P7 sequences at the other. To generate target gene specific of amplification from sc-libraries, a P7-tailed, gene-specific primer can be used together with a P5 primer. In this way, fragments comprising the gene of interest are specifically amplified and can then be used for initial sequencing based on Read 1 and Read 2 primer sequences comprised in amplified fragments. Analysis of initial sequencing results can identify the UBCs associated with cellular nucleic acid libraries from cells that expressed sequences of interest for the target gene. Selection can then be performed, followed by sequencing of desired samples.

In some embodiments, targeted initial sequencing identifies 16s rRNA sequences associated with bacterial taxa or species of interest. In some embodiments, targeted initial sequencing identifies cells in a cancer biopsy comprising KRAS G12 genes expressing mutations. After initial sequencing and identification of desired samples, the desired samples could be enriched or unwanted sample depleted. The selected cellular nucleic acid libraries could be used for deeper sequencing or whole genome analysis to better understand the sequences of single cells of interest.

Similar approaches could be used with any genes of interest. Further, initial sequencing can assay mRNA expression levels or methylation status at differential regions of the target nucleic acid to catalog cell types that corresponding different barcodes. When epigenetic factors are assessed in initial sequencing, the resequencing can then provide comprehensive whole genome sequencing of cells of the desired phenotype.
2. Representative Sequencing Information Obtained from Initial Sequencing

In these methods, the initial sequencing may provide sequence information for sorting based on an “omic” feature. In some embodiments, the initial sequencing provides information on genomic features, such as sequence or variants of one or more genes. In some embodiments, DNA from samples is sequenced to generate genomic data. In some embodiments, the initial sequencing provides information on transcriptomic features, such as expression of different genes. In some embodiments, RNA from samples is sequenced to generate transcriptomic data. In some embodiments, the initial sequencing provides data on methylation marks or patterns. In some embodiments, DNA from samples is used for methylation analysis. In some embodiments, the methylation analysis is bisulfite sequencing. In some embodiments, single cells can be sorting and then samples from the single cells can be used for bisulfite sequencing and methylation analysis. For any of these initial sequencing methodologies, the sequencing may be whole-genome or targeted sequencing.

In some embodiments, the initial sequencing is used for generating metagenomics data. In some embodiments, initial sequencing is used to identify species within a mixed pool of samples comprising samples from a number of species. In some embodiments, initial sequencing is used to identify abundant species within a mixed pool of samples comprising samples from a number of species. Resequencing may then generate further sequencing data on desired species. In some embodiments, the species are species of bacteria. In some embodiments, a mixed pool of samples comprises a mixed pool of bacteria isolated from a patient.

The initial sequencing data could be analyzed with any bioinformatics approach. Analysis of the initial sequencing results will depend on how the user wants to use the method. In other words, the user could select the most appropriate way to analyze the initial sequencing results, based on how they want to characterize the samples into desired and unwanted samples. For example, the user would use analysis of methylation status if they want that to be the criteria for selection.

Further, one distinct advantage of the present method is that the initial sequencing can be an unbiased analysis of the mixed population followed by resequencing of desired samples that are determined via the initial sequencing. For example, a user may have a metagenomics sample from an ill patient with an infection, but the user may not have any information on the bacterial species that are comprised in the sample. Using the present method, initial 16s sequencing could identify bacterial species in the sample, and the user could identify samples from bacterial species that are known pathogens. The desired samples in this case would be these potentially pathogen bacterial species, while the unwanted samples could be abundant species in the sample that are known to be non-pathogenic. Resequencing could then be performed to provide more information on the desired samples, such as whether the potentially pathogenic bacteria express genes related to resistance to antibiotics. These results could then be used to determine the best antibacterial therapy for the subject. This method is especially powerful because the user does not have to make any predictions on the presumed pathogenic species, which could bias the results if the infection is by rare bacteria. Such a methodology could also be especially useful to assess samples wherein the pathogenic bacteria is one that does not culture well. In such a case, the present method could allow identification and clinically relevant assessment of the potentially pathogenic bacteria, while a culture-based method of assessing the same patient sample would miss the presence of these unculturable pathogenic bacteria.
3. Amplification and Resequencing

In some embodiments, the method comprises one or more amplification steps after the initial sequencing. In some embodiments, the method comprises an amplification step before resequencing.

In some embodiments, amplification is used for selection. In some embodiments, desired samples are enriched via PCR amplification of desired samples using unique sample barcodes, as will be discussed below.

In some embodiments, amplification is performed after selection. In some embodiments, desired samples are enriched or unwanted samples are depleted before an amplification step. In such cases, the amplification may be unbiased and all the remaining samples in the library after selection are amplified. In some embodiments, the amplification step uses universal primers.

In some embodiments, the amplification and resequencing steps are repeated once. In some embodiments, the amplification and resequencing steps are repeated more than once. In some embodiments, the amplification and resequencing steps are repeated 1, 2, 3, 4, 5, 6, 7, 8, 9, 10, 15, 20, 25, 30, 35, 40, 45, 50, 55, or more times or any interval created from the listed integers.

In some embodiments, samples are amplified on a solid support.

C. Samples

In some embodiments, the method comprises initially sequencing a library comprising a number of individual nucleic acid libraries generated from a mixed pool of nucleic acid samples.
1. Mixed Pool of Samples

A mixed pool of samples can be any non-homogenous group of samples. For example, a mixed pool of samples could be a blood sample comprising different individual cells, a tissue sample comprising different individual cells (i.e., a tumor sample), or an environmental sample comprising different bacterial species, etc.

In some embodiments, the mixed pool of samples comprises a mixed pool of cells, a mixed pool of nuclei, or a mixed pool of high molecular weight DNA (HMW DNA). In some embodiments, the samples are cells, nuclei, or HMW DNA. In some embodiments, the HMW DNA is viral DNA. High molecular weight DNA comprises mean fragment lengths of 20 kb or higher. In some embodiments, the DNA comprises mean fragment lengths of 25, 30, 35, 40, 45, 50 kb or higher.

In some embodiments, a single sample is a single cell. In some embodiments, a plurality of nucleic acid samples from a mixed pool is a plurality of nucleic acids from a mixed pool of cells.

In some embodiments, a mixed pool of samples is collected from a patient. In some embodiments, the mixed pool is from a blood or other tissue sample or a biopsy sample taken from a tumor.

In some embodiments, a mixed pool of samples is an environmental sample. In some embodiments, the mixed pool is from a mixed pool of different species of bacteria or other microorganisms.

In some embodiments, a mixed pool of samples comprises both desired samples and undesired samples.
2. Desired Samples

As used herein, a “desired sample” refers to a sample that one skilled in the art wishes to evaluate. By this definition, it is not meant that a desired sample itself is desired, as the user may want to study malignant cells, etc., that are detrimental to the subject who is being evaluated.

For example, one skilled in the art may only be interested in certain individual cell libraries within a plurality of single cell libraries. A user may want to study cells with certain ‘omic’ profiles, such as studying cells expressing a gene mutation that confers resistance to a cancer drug treatment. Using the present method, one skilled in the art could monitor a patient for potential evolution of resistance to a certain drug treatment.

In many cases, the desired samples are comprised within a pool of samples comprising other samples that are unwanted (i.e., not desired). A desired sample may be a sample with a certain profile, wherein the desired sample is within a pool of samples including unwanted sample. For example, a desired sample may express a certain gene mutation that is not expressed by unwanted samples from the mixed pool of samples. Alternatively, a desired sample may be pathogenic bacteria that is comprised in a sample also comprising abundant non-pathogenic bacteria.

In the methods described herein, any feature that can be analyzed with sequencing may be used for characterizing a desired sample. Thus, an advantage of the present method is that it can be used with a wide range of different samples.

In some embodiments, the desired sample is a cell or nucleus. In some embodiments, the desired sample is a cell. In some embodiments, the desired sample is a nucleus from a cell.

In some embodiments, the desired sample is a human cell or a nucleus from a human cell. In some embodiments, the desired sample is a cancer cell or a nucleus from a cancer cell. In some embodiments, the desired cell or nucleus is or is from a specific desired cell type. In some embodiments, the desired sample has a mutation relative to other sample in the pool. In some embodiments, the desired sample is or is from a cancer cell or an immune cell.

In some embodiments, the desired sample is or is from a cancer cell. In some embodiments, the desired sample is or is from a cancer stem cell. In some embodiments, the desired sample is or is from a cancer cell in a liquid or tumor biopsy sample. In some embodiments, the desired sample is or is from a cancer cell resistant to drug treatment.

In some embodiments, the desired sample is or is from a cancer cell that has at least one mutation relative to other cancer cells in the pool of cells. In some embodiments, the method is used for tracking cancer evolution. In some embodiments, the cancer evolution may be the emergence of resistance to a given chemotherapy treatment. In some embodiments, the desired sample is or is from a cell having a somatic driver mutation.

In some embodiments, the desired sample is a metagenomics sample. In some embodiments, the desired sample is a microbe from an environmental sample. In some embodiments, the desired sample is a microbe that is not cultured from an environmental sample. In some embodiments, the microbe comprises bacteria, fungi, archaea, fungi, algae, protozoa, or virus. In some embodiments, the desired sample is a pathogen.

In some embodiments, the desired sample has a mutation in its nucleic acid compared to other samples. In some embodiments, the desired sample has a single nucleotide variant (SNV). In some embodiments, the desired sample has a copy number variation (CNV).

In some embodiments, the desired sample has a desired methylation pattern. In some embodiments, the desired sample has a desired expression pattern. In some embodiments, the desired sample has a desired epigenetic pattern. In some embodiments, the desired sample has a desired immune gene recombination.

In some embodiments, the sample has a specific species type. In some embodiments, the specific species type is a human species. In some embodiments, the specific species type is a specific species of bacteria.

Some representative uses of the present methods with different types of samples are described below.
a) Rare Samples

In some embodiments, the desired samples are rare within the starting population. For example, the desired sample may be that from single cells that were rare in the population of cells used to generate a sc-library. As such, desired sequencing data from rare cells could be overwhelmed by the sequencing data from abundant unwanted cells, if sequencing data from the entire pool of libraries from individual cells in a mixed pool of cells is evaluated.

As used herein, a desired sample is a “rare sample” that is present in less than or equal to 1%, 0.1%, 0.01%, 0.001%, 0.0001%, 0.00001%, 0.0000010%, 0.00000010%, 0.000000010%, or 0.0000000010% of a mixed pool of samples. In some embodiments, the desired sample is a desired cell. In some embodiments, a desired cell is present in less than or equal to 1%, 0.1%, 0.01%, 0.001%, 0.0001%, 0.00001%, 0.000001%, 0.0000001%, 0.00000001%, or 0.000000001% of a mixed pool of cells. A rare cell may be characterized by any feature that can be evaluated by an initial sequencing, such a feature based on a cell's genome or epigenetic makeup. For example, a rare cell may be one wherein its DNA comprises a mutation compared to the DNA of the other cells in the sample. In some embodiments, a rare cell may be one wherein the methylation pattern of its DNA is different compared to other cells in the sample. In the methods described herein, any feature that can be analyzed with sequencing data may be used for characterizing a rare sample.

In some embodiments, initial sequencing in the present method can be used to identify libraries produced from rare cells. A selection step can be performed to enrich desired samples (i.e., libraries from rare cells of interest) or deplete unwanted samples (i.e., libraries from abundant unwanted cells). After the selection, the resulting library can be resequenced by deeper sequencing to evaluate the characteristics of the desired rare cells.
3. Unwanted Samples

As used herein, an “unwanted sample” refers to a sample that one skilled in the art does not want to sequence. An unwanted sample may be a beneficial cell, but not of interest to the user. For example, a user may want to evaluate liver cancer cells from a biopsy, but not evaluate cells comprising normal non-cancerous liver tissue. One skilled in the art may also only want to sequence samples from cells expressing a certain genetic mutations and not want to sequence samples from other cells in a sample. Without selection to enrich desired samples or deplete unwanted samples, sequencing of unwanted samples can waste time, resources, and sequencing capacity.

D. Nucleic Acids

These methods can be used to evaluate nucleic acids. In some embodiments, these nucleic acids are from single cells. In some embodiments, the nucleic acid is DNA. In some embodiments, the nucleic acid is RNA. In some embodiments, the nucleic acid is ribosomal RNA (rRNA). In some embodiments, the nucleic acid is 16s rRNA. In some embodiments, the nucleic acid is 18s rRNA.

In some embodiments, the nucleic acid is ribosomal DNA (rDNA).

In some embodiments, the nucleic acid is internal transcribed spacer nucleic acid.

E. Unique Sample Barcodes and Unique Cellular Barcodes

As used herein, a “unique sample barcode” refers to a barcode that is unique for an individual sample within a pool of samples. In some embodiments, initially sequencing a library comprises sequencing a library comprising a plurality of nucleic acid samples from a mixed pool of samples. This mixed pool of samples can be any non-homogenous group of samples, such as a blood sample comprising different individual cells. In some embodiments, a unique sample barcode can distinguish the nucleic acids from the desired single sample from the nucleic acids from other samples in the library.

A unique sample barcode may be comprised of a single barcode sequence. Alternatively, a unique sample barcode may be comprised of multiple barcode sequences. As used herein, a “barcode sequence” refers to a sequence that may be used to differentiate samples. For example, a unique sample barcode may be unique to a given desired sample within a mixed pool of samples based on multiple barcodes that are comprised in the unique sample barcodes, even if a given barcode sequence may be associated with multiple samples. In such a case, the specific combination of barcode sequences within the unique sample barcode may be unique, although one or more barcode sequence within the unique sample barcode is shared with other samples.

In some embodiments, a unique sample barcode is a unique cellular barcode. As used herein, a “unique cellular barcode” or “UBC” refers to a barcode that is unique for a single cell within a mixed pool of cells. When analyzing sequencing data, a UBC may be used to identify sequences that were originally comprised in the same single cell within the starting mixed pool of cells.

In some embodiments, a unique sample barcode is unique for a type of nuclei, HMW DNA, etc., and the present invention is not limited to uses with single cells.

To enable a robust enrichment method, certain unique sample barcode designs may be desirable. For instance, if using a hybrid capture approach, enrichment specificity will depend on the ability to design probes to uniquely hybridize to desired unique sample barcodes. Similar consideration is true for unique sample barcode-targeting PCR amplification. For this it may be desirable to have the unique sample barcode present as a contiguous nucleic acid sequence appended to cellular DNA libraries. Alternatively, it may be desirable to have fixed sequences between barcode sequences in a unique sample barcode, such that the user knows primers that will bind to bind to combinations of barcode sequences within a unique sample barcode.

Unique sample barcodes may be used in combination with other known barcodes or adaptor sequences. For example, library fragments may comprise unique sample barcodes and also comprise one or more commercially available adaptors. In some embodiments, i5 and/or i7 adaptor sequences (Illumina) are comprised in library fragments.
1. Types of Barcodes

In some embodiments, a barcode is a physically adressable barcode. By “physically addressable,” it is meant that the barcode comprises one or more nucleic acid sequences that can bind another agent. In some embodiments, the physically addressable barcode can bind a complementary nucleic acid sequence. In some embodiments, the physically addressable barcode can be bound by a primer or a capture oligonucleotide. For example, a physically addressable barcode may bind to a sequencing primer to allow sequencing of a library fragment. In another example, a physically addressable barcode may bind to a capture oligonucleotide to allow immobilization of a library fragment on a flow cell.

In some embodiments, a barcode is a unique sample barcode.

In some embodiments, the unique sample barcode is a single contiguous barcode. In some embodiments, the unique sample barcode comprises more than one barcode sequence, without nucleic acid sequences between the different barcode sequences. For example, multiple barcode sequences (BC₁-BC_{X}) can be added in different steps, wherein no nucleic acid sequence is incorporated between the barcode sequences. As shown in the exemplary method of FIG. 5, BC₁ can be incorporated during tagmentation, and BC₂-BC_{X} can be incorporated via ligation. As shown in the exemplary method of FIG. 6, BC₁ can be incorporated during tagmentation, followed by one or more rounds of ligation of well-specific BC's followed by pooling. Preparation of a single contiguous barcode can allow ease in designing a primer that can bind to the unique sample barcode.

In some embodiments, the unique sample barcode is multiple discontiguous barcodes. In some embodiments, the multiple discontiguous barcodes are separated by nucleic acid sequences. In some embodiments, the multiple discontiguous barcodes are separated by fixed sequences. For example, multiple barcode sequences (BC₁-BC_{X}) can be added in different steps, wherein nucleic acid sequence is incorporated between the barcode sequences. Such multiple discontiguous barcodes can allow ease in designing a primer that can bind to the unique sample barcode because the barcodes and fixed sequences are known.

F. Endonucleases

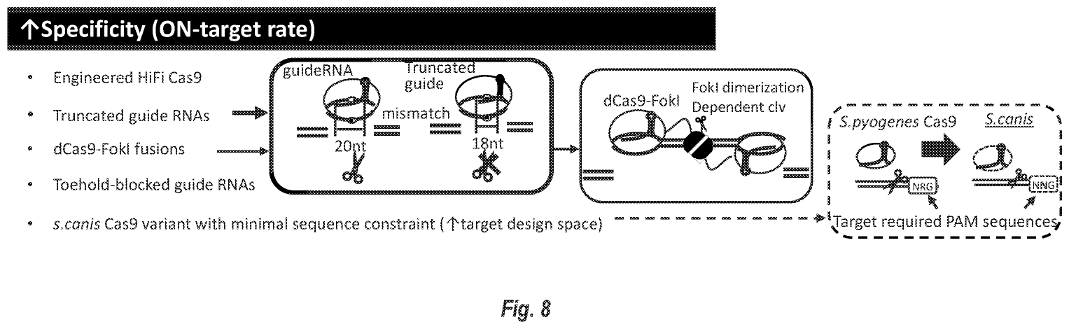

Different endonucleases may be used in the present methods. As used here, the term “endonuclease” is used to refer an enzyme that can cleave a nucleic acid. An endonuclease can refer to either a catalytically active endonuclease or a catalytically inactive endonuclease. Some features of endonucleases, such as an ability to target to a specific target sequence based on a guide RNA associated with the endonuclease, are common to both catalytically active and catalytically inactive endonucleases. In some embodiments, an endonuclease is associated with a guide RNA that binds to one or more unique sample barcode. A variety of different endonucleases that may be used to improve specificity (i.e., to improve targeting and reduce off-target activity) are presented in FIG. 8.

In some embodiments, the endonuclease is a catalytically inactive endonuclease. As used herein, “catalytically inactive endonucleases” are endonucleases that can bind nucleic acid but do not mediate nucleic acid cleavage. A catalytically inactive endonuclease may also be referred to as a deactivated endonuclease (such as a “dCas” protein). An exemplary catalytically inactive endonuclease is dCas9, as shown in FIG. 3 (wherein the dCas9 is bound to biotin) and FIG. 8 (wherein the dCas9 is comprised in a fusion protein with FokI). Normally, an endonuclease can bind to a nucleic acid and then mediate cleavage. Thus, a catalytically inactive endonuclease is one that retains nucleic acid binding function, without having cleavage activity. Catalytically inactive endonucleases may be used for selection steps of the present methods. In some embodiments, a catalytically inactive endonuclease is used for depleting unwanted samples. In some embodiments, a catalytically inactive endonuclease is used for enriching desired samples. In some embodiments, a catalytically inactive endonuclease is bound directly or indirectly to a solid support. In some embodiments, a catalytically active endonuclease is bound to a solid support through a biotin-streptavidin interaction.

Further, one skilled in the art would be aware of catalytic domains of endonucleases and could design a mutation to generate catalytically inactive endonuclease from a wildtype endonuclease (See Maeder et al., Nat Methods 10(10): 977-979 (2013)). Such a designed catalytically inactive endonuclease could be tested to confirm its lack of cleavage activity. Representative catalytically inactive Cas9 proteins include those disclosed in U.S. Ser. No. 10/457,969, which is incorporated herein in its entirety.

In some embodiments, the endonuclease is a catalytically active endonuclease, meaning it can cleave nucleic acid. In some embodiments, a catalytically active endonuclease is used for depleting unwanted samples.

In some embodiments, an endonuclease is associated with a guide RNA. An endonuclease can be targeted to one or more nucleic acid sequence of interest by a guide RNA. In some embodiments, the nucleic acid sequence of interest is one or more unique sample barcodes.

In some embodiments, an endonuclease has minimal PAM specificity (as shown in FIG. 8) that allows greater flexibility in designing guide RNAs.

In some embodiments, an endonuclease is associated with a guide RNA that binds to one or more unique sample barcodes. In some embodiments, guide RNAs are directed against unique sample barcodes associated with nucleic acids of unwanted samples. In some embodiments, guide RNAs are directed against unique sample barcodes associated with nucleic acids of desired samples.

In some embodiments, the endonuclease is from cyanobacteria Scytonema hofmanni (ShCAST). ShCAST is a 4-protein system for RNA-directed (sgRNA) DNA-transposition mediated by Tn7-like transposase subunits and the type V-K CRISPR effector (Cas12k) (See Strecker et al., Science. 365(6448): 48-53 (2019), including the embodiment shown in FIG. 5 of Strecker). Other systems wherein Tn7-like transposons have co-opted nuclease deficient CRISPR-Cas systems to generate a CRISPR-associated transposase have also been described (See Klompe et al., Nature 571:219-225 (2019)).

A number of different means to increase the specificity of an endonuclease are shown in FIG. 8. The methods described herein could use any type of endonuclease and/or guide RNA that may improve specificity. In some embodiments, the improved specificity of an endonuclease is due to improved binding of an endonuclease to one or more unique sample barcodes. Such improved binding may be a higher percentage of binding to one or more unique sample barcodes of interest (i.e. specific binding) compared to binding to other sequences (i.e. non-specific binding).

In some embodiments, a catalytically active endonuclease is an endonuclease that has greater specificity for cutting a nucleic acid. In some embodiments, this greater specificity is not due solely to greater specificity in binding to a target sequence in a nucleic acid. In some embodiments, these catalytically active endonucleases with greater specificity can cleavage unwanted samples and deplete them from the sample.

In some embodiments, a catalytically active endonuclease is a higher-fidelity mutant. A “higher-fidelity” endonuclease refers to one with reduced off-target activity compared to a wildtype endonuclease.

In some embodiments, a catalytically active endonuclease is comprised in a fusion protein together with FokI nuclease. In some embodiments, the fusion protein comprises Cas9 and FokI nuclease (See Guilinger et al., Nat Biotechnol. 32(6): 577-582 (2014)). Such a fusion protein may work to require binding of two separate fusion proteins comprising a catalytically inactive Cas9 fused to a FokI nuclease (as shown in FIG. 8) in close proximity, after which the dimerized FokI nucleases can cleave the target nucleic acid. In some embodiments, the two fusion proteins bind to different target sequences. In some embodiments, the two fusion proteins bind to two different unique sample barcodes.

G. Enriching

A number of different methods of enriching may be used to select the desired samples, while not selecting the unwanted samples. In this way, only the desired samples are resequenced, without resequencing the unwanted samples.

In some embodiments, the depleting refers to physically separating unwanted samples from desired samples. In some embodiments, depleting comprising capturing desired samples on a solid support and discarding uncaptured sequences. Such a capture step could avoid capture of unwanted samples, and the unwanted samples would be discarded. After such an enriching step, only desired samples would remain within the library.

In some embodiments, the enriching step comprises hybrid capture, unique sample barcode-specific amplification, or capture via a catalytically inactive endonuclease. In some embodiments, the unique sample barcode is used to direct enrichment of desired samples. In some embodiments, the unique sample barcode is used to direct enrichment of desired samples from one or more single cells from a mixed pool of cells.

In some embodiments, multiple steps of enrichment are performed. In some embodiments, the multiple steps comprise the same type of enrichment. For example, two or more hybrid capture steps are performed, wherein different hybrid capture oligonucleotides may be used for different steps.

In some embodiments, multiple steps of enrichment comprise different types of enrichment. For example, an enrichment by hybrid capture may be performed, followed by a PCR amplification.

In some embodiments, sequencing may be performed between multiple enrichment steps. Such sequencing results can indicate what desired samples should be further enriched.

In some embodiments, selection is performed by combining enrichment and depletion steps. In other words, any combination of selection steps described herein may be combined by the user.
1. Hybrid Capture

In some embodiments, the enriching step comprises hybrid capture. In some embodiments, the hybrid capture step comprises hybridizing a hybrid capture oligonucleotide to a unique sample barcode. This step may be performed with a number of hybrid capture oligonucleotides that bind to a set of unique sample barcodes, wherein the unique sample barcodes represent the unique sample barcodes of a number of desired samples. For example, the initial sequencing data may indicate that a set of single cells in the mixed pool of cells express a given gene mutation, and the unique sample barcodes associated with these single cells may be used for hybrid capture to enrich for nucleic acid libraries from these particular single cells. After the enrichment, resequencing can be performed to generate additional sequencing data on single cells of interest. This method could avoid generating additional sequencing data on unwanted cells, as samples from the unwanted cells would not be enriched during the hybrid capture step.

In some embodiments, the unique sample barcodes are selected to hybridize with a known panel of hybrid capture oligonucleotides. Alternatively, a custom panel of hybrid capture oligonucleotides may be generated based on the unique sample barcodes used when preparing the nucleic acid libraries.

In some embodiments, the hybrid capture oligonucleotide is bound to an affinity element. In some embodiments, the affinity element is used to allow capture of oligonucleotides that are bound to certain unique sample barcodes, to allow enrichment of libraries comprising these unique sample barcodes. In some embodiments, the affinity element is biotin. A range of affinity elements would be known those skilled in the art, such magnetic microparticles that could be bound by certain capture beads.

In some embodiments, the hybrid capture oligonucleotide is bound directly or indirectly to a solid support. In some embodiments, the hybrid capture oligonucleotide is bound to a solid support through a biotin-streptavidin interaction. In some embodiments, the solid support is a bead.
2. Capture Via Catalytically Inactive Endonucleases

In an analogous fashion to hybrid capture, catalytically inactive endonucleases associated with specific guide RNAs can be used for enrichment. These catalytically inactive endonucleases can be targeted to specific unique sample barcodes using guide RNAs. In some embodiments, capture via catalytically inactive endonucleases comprises binding the catalytically inactive endonucleases to the unique sample barcode via guide RNAs.

In some embodiments, the catalytically inactive endonuclease is bound to an affinity element. In some embodiments, the affinity element is used to allow capture of catalytically inactive endonucleases that are bound to certain unique sample barcodes, to allow enrichment of libraries comprising these unique sample barcodes. In some embodiments, the affinity element is biotin. A range of affinity elements would be known those skilled in the art, such magnetic microparticles that could be bound by certain capture beads.

In some embodiments, the catalytically inactive endonuclease is bound directly or indirectly to a solid support. In some embodiments, the catalytically inactive endonuclease is bound to a solid support through a biotin-streptavidin interaction. In some embodiments, the solid support is a bead.
3. PCR Amplification

In some embodiments, enrichment is via PCR amplification. In some embodiments, enrichment is by unique sample barcode-targeting PCR amplification. In some embodiments, primers that bind to certain unique sample barcodes allow amplification of desired samples, based on the unique sample barcodes known to be associated with desired samples from the initial sequencing. In contrast, primers that bind to other unique sample barcodes associated with unwanted samples would not be included in the amplification reaction. In this way, the desired samples could be selected.

H. Depleting

A number of different methods of depleting may be used to remove unwanted samples, while not removing the desired samples. In this way, only the desired samples are resequenced, without resequencing the unwanted samples.

In some embodiments, the depletion step comprises hybrid capture, capture via catalytically inactive endonucleases, or CRISPR digestion.

In some embodiments, unique sample barcodes are used to direct depletion of unwanted samples. In some embodiments, unique sample barcodes are used to direct depletion of unwanted samples from one or more single cells from a mixed pool of cells.

In some embodiments, multiple steps of depletion are performed. In some embodiments, the multiple steps comprise the same type of depletion. In some embodiments, the multiple steps of enrichment comprise different types of depletion. For example, a depletion by hybrid capture may be performed, followed by a CRISPR digestion. In some embodiments, sequencing may be performed between depletion steps. For example, a method may comprise initial targeted sequencing, depletion of unwanted samples, another targeted sequencing, depletion of additional unwanted samples, and a comprehensive resequencing.
1. Depleting by Physically Separating Unwanted Samples from Desired Samples

In some embodiments, the depleting refers to physically separating unwanted samples from desired samples. In some embodiments, depleting comprising capturing unwanted samples on a solid support and removing them. After such a depleting step, only desired samples would remain within the library.

In some embodiments, hybrid capture may be performed as described for enriching of desired samples, except that unwanted samples isolated by hybrid capture are then removed from further resequencing (instead of being retained for resequencing as was the case for desired samples in enriching embodiments).

In some embodiments, capture via catalytically inactive endonucleases capture may be performed as described for enriching of desired samples, except that unwanted samples isolated by capture via catalytically inactive endonucleases are then removed from further resequencing (instead of being retained for resequencing as was the case for desired samples in enriching embodiments).
2. Depleting by Cleavage of Unwanted Samples

In some embodiments, the depleting comprises cleavage that makes an unwanted sample unable to be properly sequenced. In other words, the depleting may refer to making an unwanted sample have less or no ability to be properly sequenced based on cleavage of the sample. In some embodiments, nucleic acid from unwanted samples is within the library and selection, but the depleting refers to a decreased ability of these unwanted samples to be sequenced.

For example, cleavage of a sequence within or near one or more unique sample barcodes associated with an unwanted sample could separate off a nucleic acid sequence necessary for sequencing from the rest of the unwanted sample. In such a way, this unwanted sample would no longer be able to generate sequencing results in a resequencing after depletion. In some embodiments, such a cleavage separates a nucleic acid sequence from the rest of the unwanted sample. In some embodiments, the nucleic acid sequence separated is an adapter sequence. In some embodiments, such an adapter sequence could be a primer sequence or a sequence for immobilizing nucleic acids to a flow cell used for sequencing. For example, separating a sequencing primer binding site from the rest of an unwanted sample could make the unwanted sample incapable of being sequenced via a chosen sequencing method. One skilled in the art could identify such sequences that could be separated to mediate depletion, based on the platform used for sequencing and the composition of the library originally generated.

In some embodiments, the depletion step comprises CRISPR digestion. As used herein, CRISPR (clustered regularly interspaced short palindromic repeats) refers to a family of DNA sequences found in the genomes of prokaryotic organisms such as bacteria and archaea. As used herein, CRISPR digestion refers to any digestion of one or more nucleic acid based on a CRISPR sequence. Endonucleases, such as Cas9, can utilize CRISPR sequences to cleave a nucleic acid at defined sequences. In some embodiments, the endonuclease is a catalytically active endonuclease.

In some embodiments, CRISPR digestion is directed against unique sample barcodes associated with nucleic acids of unwanted samples. In some embodiments, CRISPR digestion comprises cleavage of unwanted samples. In some embodiments, CRISPR digestion separates a nucleic acid sequence necessary for sequencing from the rest of an unwanted sample to deplete the unwanted sample.
a) Methods of Cleavage of Unwanted Samples with ShCAST

In some embodiments, methods of depleting are performed using cleavage with ShCAST. In some embodiments, cleavage renders unwanted samples unable to be amplified and/or sequenced.

In some embodiments, the ShCAST comprises Cas12K; the transposase comprises Tn5 or a Tn7-like transposase; and/or at least one of the gRNA and the transposase is biotinylated, wherein at least one of the gRNA and transposase that is biotinylated is capable of coupling to a streptavidin-coated bead. In some embodiments, a biotinylated gRNA and/or transposase allows for capture of unwanted samples to streptavidin beads. In this way, unwanted samples can be removed from a reaction mixture while retaining desired samples.

In some embodiments, a fluid (also known as a reaction fluid) is used that limits binding of the transposase comprised in the ShCAST. In some embodiments, limiting or inhibiting binding of the transposase reduces off-target transposition reactions mediated by the transposase comprised in ShCAST. When off-target cleavage is reduced, the depleting step can be more selective for only depleting unwanted samples without affecting desired samples.

In some embodiments, depleting nucleic acid samples from unwanted samples is performed in a fluid having a condition for limiting cleavage by the complex. One skilled in the art would be aware of a number of means to limit cleavage by a transposition reaction mediated by a transposase, and any means known in the art can be employed. For example, transposase activity is dose-dependent (i.e., a lower concentration of transposase limits the number of transposition reactions). In addition, transposases are magnesium-dependent. In some embodiments, the condition for limiting cleavage by the complex is a magnesium concentration of 15 mM or lower and/or with a concentration of Cas12K and/or transposase of 50 nM or lower.

In some embodiments, cleavage of a nucleic acid by ShCAST allows for timing of steps. For example, a user may wish to limit binding and/or cleavage of the nucleic acid by ShCAST in initial reaction steps to allow for greater selectivity (e.g., cleaving unwanted samples and not desired samples). In later reaction steps, a user may wish to promote cleavage of the nucleic acid by the transposase comprised in the complex for efficient cleaving of unwanted samples. In other words, a user may want binding of the transposase to be relatively selective, while cleavage of the nucleic acid by the transposase to occur with relatively high efficiency. Thus, initial conditions during hybridizing of complexes to a nucleic acid may inhibit binding of a transposase comprised in a complex to nucleic acid and/or inhibit cleavage by the transposase comprised in the complex. Later conditions of a method may promote cleavage of the nucleic acid by the transposase.

In some embodiments, depleting nucleic acid samples from unwanted samples comprises (1) binding complexes to the double-stranded nucleic acid under conditions that inhibit cleavage of the nucleic acid by the complex and (2) after the binding, promoting cleavage of the nucleic acid by the complex.

In some embodiments, the binding is performed under conditions that (1) inhibit binding of the complex to a target nucleic acid and (2) inhibit cleavage of the target nucleic acid by the complex. In other words, initial conditions may inhibit both binding of the complex and inhibit cleavage by the complex.

In some embodiments, different means of selective activation of transposases may be used. In some embodiments, during binding, transposases comprised in ShCAST are inactive or less active based on reaction conditions used. In some embodiments, reaction conditions are modified after binding of ShCAST to nucleic acid, allowing for a high efficiency of cleavage by the transposase after more selective binding of ShCAST. In such embodiments, reversibly deactivated transposases may be used, wherein the user can control the time at which transposases are active by using a step of selective activation. While such means of selective activation of transposases are described for ShCAST, these methods can be used with other methods incorporating transposases.

In some embodiments, a transposase is reversibly deactivated during the binding and promoting cleavage comprises activating the transposase.

In some embodiments, the magnesium concentration is low (e.g., less than 15 mM) during the binding, and promoting cleavage comprises increasing the magnesium concentration.

In some embodiments, a transposase is absent during the binding, and promoting cleavage comprises adding a transposase.

In some embodiments, the transposase is reversibly deactivated due to lack of one or more transposon, and activating the transposase comprises providing one or more transposons.

### VII. Representative Uses of Methods

The present methods could be used in a variety of sequencing applications. The specific uses described herein are not meant to limit the invention, as one skilled in the art could envision a wide range of ways that the present methods could be used to improve results of various sequencing applications.

A. Corrective Library Quality Control

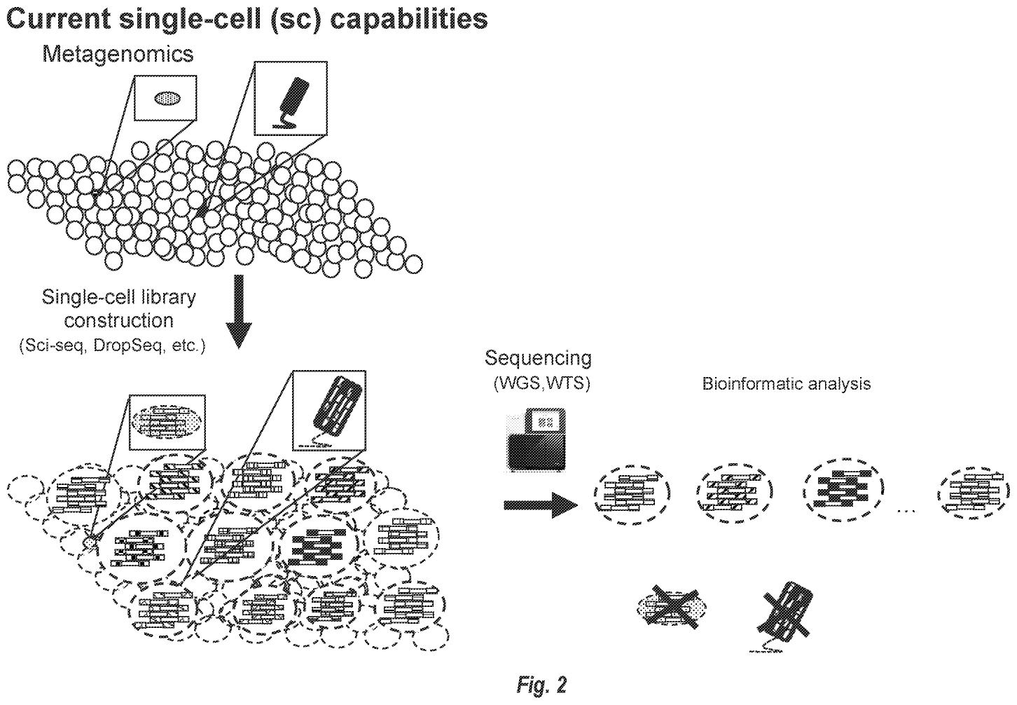

In some embodiments, the present methods may be used for quality control (QC) of a library comprising a plurality of nucleic acid samples from a mixed pool of samples. In some embodiments, the enriching or depleting step is used for quality control. In some embodiments, a quality control step is corrective, in that it reduces signal from unwanted samples. FIG. 2 provides overview of how current single-cell methods, without quality control steps described herein, may lose information from rare cells from metagenomics samples.

As used herein, “quality control” or “QC” refers to a selection step that is based on the nature of the resulting libraries from various individual within a library, and not based on a factor related to the original mixed population of samples. In other words, QC methods do not necessarily identify desired samples or unwanted samples of single cell libraries based on a biologic difference between the samples in the original mixed pool of samples used to generate the library, but instead identify desired samples or unwanted samples based on a factor related to the library produced.

For example, a given library produced from a single cell may be of lower quality based on a random difference in the process of library generation, and not based on a biological difference between this cell and other cells in the original mixed pool of cells. Unwanted samples could include those single cell libraries with insufficient numbers of fragments, those with fragments of undesired size, etc. Any factor that might reduce the quality of sequencing results could lead to a particular nucleic acid library being classified as an unwanted sample. In other words, one skilled in the art can correct a sub-standard library preparation (where some samples associated with unique sample barcodes are noise and scattered) using the present method, and the unwanted samples are removed from the library and then resequencing is performed. This resequencing may then be focused on those libraries that can potentially produce sequencing data of sufficient quality.

In some embodiments, the initial sequencing identifies the desired libraries and the unwanted libraries based on the quality of the sequencing results.

In some embodiments, an initial sequencing reaction identifies unique sample barcodes associated with libraries of single cells that are unwanted samples, due to these libraries being of lower quality. In some embodiments, unwanted samples of libraries are identified by initial sequencing, and these libraries are depleted from the sc-library before resequencing. In some embodiments, desired samples of libraries are identified by initial sequencing to identify libraries of higher quality, and these libraries are enriched from the sc-library before resequencing.

In some embodiments, the quality control step increases the quality of the library used for resequencing. In this way, the resequencing can focus on deeper sequencing of higher-quality libraries. In some embodiments, a QC step can avoid a waste of time and reagents by avoiding deeper sequencing of lower-quality libraries (i.e., the unwanted samples).

B. Oncology Uses

In some embodiments, the present methods are used to evaluate or monitor disease. In some embodiments, the disease is cancer.

In some embodiments, the cancer is a blood or solid tumor. In some embodiments, the cancer can be evaluated based on a biopsy from a solid tumor or a sample of blood. In some embodiments, the present method is used to evaluate a heterogeneous tumor or to evaluate circulating cancer cells (CTCs). CTCs are putative markers of tumor prognosis and may serve to evaluate a subject's response to a given treatment (such as chemotherapy or immunotherapy).

In some embodiments, the present methods are used to evaluate cells in the tumor microenvironment, which may or may not be cancer cells. These cells that are not cancer cells may be stromal cells, vascular cells, or any other type of cell that may in proximity to the cancer cells without being cancerous themselves. Cells in the tumor microenvironment are known to influence tumor growth and metastasis.

In some embodiments, an initial sequencing evaluates libraries within the sc-library via targeted sequencing for variant cells. These variant cells may be those with single nucleotide polymorphisms, insertions, deletions, and/or copy number variants in their nucleic acids. These variant cells may also have a difference in another factor or factors, such as a change in methylation. In some embodiments, these variants are CTCs. Based on the initial sequencing, a selection step can be done to enrich or deplete for variant cells, resulting in a sc-library comprising cellular nucleic acid libraries of interest. These libraries can then be used for a resequencing step for deeper genomic characterization of variant cells.

In some embodiments, initial sequencing is targeted sequencing of a somatic driver mutation region(s). A somatic driver mutation is a mutation that confers a growth advantage to cells expressing it, and these cells may be positively selected during evolution of the cancer. In some embodiments, initial sequencing assigns a cancerous/molecular type to individual cellular nucleic acid libraries tagged by a given unique sample barcode within a plurality of cellular nucleic acid libraries. In some embodiments, deeper resequencing is performed after selection of libraries tagged by unique sample barcodes associated with a driver mutation.

In some embodiments, a somatic driver mutation is a mutation in KRAS G12. In some embodiments, initial sequencing is targeted sequencing of KRAS G12. In some embodiments, analysis is performed to determine UBC barcodes of individual cellular nucleic acid libraries with KRAS G12 mutations (as shown in FIG. 7). In some embodiments, after selection for these libraries of interest, resequencing is deeper sequencing or whole genome sequencing to better understand the profile of the cells with KRAS G12. A similar protocol could be used to select for and evaluate sequencing data from cell expressing any other mutation of interest.

In some embodiments, the present methods are used to track tumor evolution. As used herein, “tumor evolution” refers to changes in the characteristics of cancer cells over time, and tracking tumor evolution may involve characterizing cellular evolution patterns. For example, tumors are heterogenous, and over time this intratumor heterogeneity allows for change in tumor characteristics as certain traits are selected for over time. Changes in tumor characteristics may allow the tumor to have faster growth or metastasis or evolve to have resistance to a given treatment.

If a subject's tumor develops resistance to a given chemotherapy, for example, treatment with this agent may no longer work to slow or stop tumor growth. Methods described herein can use selection to deeply sequence cells of interest to evaluate the existence or development of resistance to a given treatment. In this way, the subject's treatment plan can be optimized to focus on therapies that are likely to be effective for the subject and avoid therapies that are less likely to be effective.

C. Metagenomics Uses

The present methods may be used for metagenomics. As used herein, “metagenomics” refers to the study of genetic material recovered directly from environmental samples. In some embodiments, these environmental samples comprise more than one microorganism. As used herein, a microorganism may include a bacteria, virus, fungus, or other small organism. For example, a metagenomics sample may comprise a microbial community (such as a variety of bacteria).

In some embodiments, metagenomics analysis avoids cultivation of organisms. In other words, metagenomics samples may be evaluated without first culturing them to artificially grow them. Avoiding cultivation can avoid selection pressure against organisms that do not grow well in culture. Further, avoiding cultivation may be especially important if little is known about the microorganisms of interest, such as the proper cultivation conditions. Otherwise, microorganisms of interest may be selected against by the culture conditions and lost from the mixed population before sequencing as other microorganisms culture better.

With prior methods, de novo assembly and species identification of rare, uncultivable microbes is nearly impossible (See Malmstrom and Eloe-Fadrosh mSystems 4:e00118-19 (2019)). Prior approaches included separating single-amplified genomes (SAG) by cell partitioning (i.e. FACS, microfluidics), followed by cell lysis and whole genome analysis (Approach 1). Another approach was metagenome-assembled genome (MAG) analysis, short/long-read shotgun sequencing using differential binning by coverage, and analysis of tetranucleotide frequency (Approach 2). An alternative approach is a “mini-metagenome” hybrid approach (Quake lab, MetaSort) (Approach 3).

However, these approaches in the art are best-suited for assembly and species identification of abundant species in a low diversity sample. By diversity, it may mean the number of different species within the sample. In other words, the prior metagenomics methods have limited use for assembly and species identification of uncommon or rare species in a high diversity sample.

For example, Approach 1 would be tractable only with a priori knowledge of a sortable phenotype to deplete abundant species and enrich rare species. Further, the cell partitioning of Approach 1 cannot be performed in the absence of enrichable or partitionable features. In addition, all the prior art methods may be associated with prohibitive sequencing costs to completely characterize microbiome samples.

In contrast, the present method may be used to select for desired samples based on initial sequencing. These desired samples could be cellular nucleic acid libraries from microorganisms of interest within a metagenomics sample. After selection, by enrichment or depletion, resequencing could be done to provide more deeper sequencing data on these microorganisms of interest.

In some embodiments, the present methods uniquely barcode each organism's DNA (RNA) in a microbiome sample, such that it is physically addressable for enrichment of desired cellular nucleic acid libraries or depletion of unwanted cellular nucleic acid libraries after initial sequencing and analysis.

In some embodiments, the initial sequencing focuses on targeted sequencing. In some embodiments, initial sequencing is ribosomal RNA or DNA (rRNA or rDNA) sequencing. In some embodiments, initial sequencing is 16S, 18S, or internal transcribed spacer sequencing. In some embodiments, initial sequencing assigns taxa-level identification to the cell RNA/DNA tagged by a given barcode within a plurality of cellular nucleic acid libraries. In some embodiments, this targeted sequencing is prokaryotic 16s rDNA or rRNA sequencing. Sequencing of variable regions of 16s rRNA are frequently used for phylogenetic classification such as genus or species in diverse microbial populations.

In some embodiments, an initial sequencing reaction is performed followed by analysis such as determination of abundant species/taxa from 16s rDNA analysis (see FIG. 7 for an example of such targeted sequencing). For example, the initial sequencing may be 16s rRNA sequencing for all cellular nucleic acid libraries, followed by whole genome sequencing of desired cellular nucleic acid libraries after a selection step. Such a method can save time and money by focusing deep sequencing on libraries from microorganisms of interest.

In some embodiments, initial sequencing is performed using contiguity preserving transposition sequencing. In some embodiments, contiguity preserving transposition sequencing is used when the sample comprises significant amounts of intact single chromosomes or high molecular weight genomic after extraction.

In some embodiments, metagenomics may be used to evaluate a sample taken from a patient. In some embodiments, a sample may be taken from a patient who displays symptoms of an unknown infection. In some embodiments, a sample may be a microbiome sample (such as a fecal sample to assess a subject's microbiome). As used herein, a microbiome sample refers to the aggregate of microbiota that reside on or within a human tissue or biofluid.

D. Immunology Uses

In some embodiments, the present methods are used for immunological analysis. In some embodiments, the method is used to evaluate T-cell clonotypes. The composition of a given individuals T-cell clonotypes may be referred to as a T-cell repertoire. In some embodiments, the initial sequencing characterizes TCR repertoires. In some embodiments, the selection step depletes abundant T-cell clonotypes. In some embodiments, resequencing is used for deeper sequencing of uncommon T-cell clonotypes.

### EXAMPLES

### Example 1. Enrichment from a Sci-RNA3 Library or Other Sc-Library

A wide range of different means of generating a single-cell library (sc-library) are known in the art. The present method can be used with any of these different methods of generating sc-libraries, based on the specific indices comprised in library fragments.

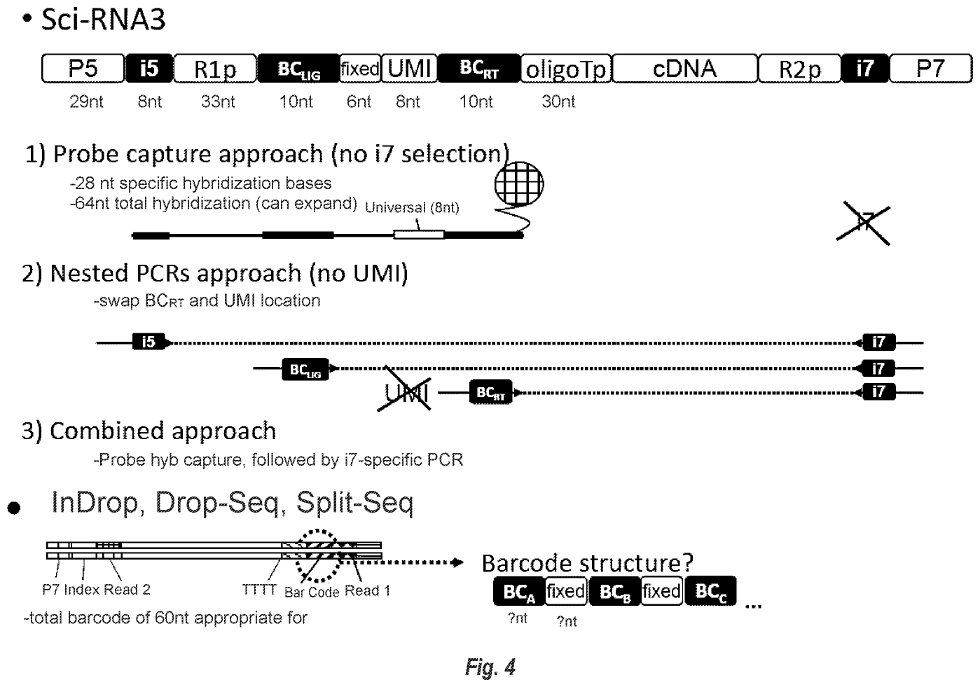

For example, a single cell sequencing library may be generated using the sci-RNA-seq3 (See Cao et al., Nature 566(7745): 496-502 (2019)), as shown in FIG. 4. This method utilizes an RT index (BCRT) and ligation adaptor index (BCLIG), along with i5 and i7 indices. The i5 and i7 indices are commercially available sets of 96 unique adaptors (Illumina).

The RT index can be combined with hairpin adaptor index (oligoTp). The multiple indices allow for demultiplexing of reads, such as removing duplicates based on reads having identical UMI, RT index, ligation adaptor index, and tagmentation site. FIG. 4 shows the different indices (i.e., barcodes) used as black ovals: BCRT (10 nucleotides), BCLIG (10 nucleotides), i5 (8 nucleotides), and i7.

A variety of different means could be used for enrichment together with a sc-library generated by a sci-RNA-seq3 method (Sci-RNA3).

First, a probe capture approach may be used that avoids i7 selection. Based on the nucleotides comprised in the i5, BCLIG, and BCRT indices, a total of 28 bases represent specific hybridization bases for developing capture probes, with a total of 67 nucleotides available for hybridization (including the 33 nucleotides of RI primer and 6 nucleotides of the fixed region). In this calculation, capture probes would comprise a universal sequence for binding to the UMI sequence.

Second, a nested PCR approach may be used. In this approach, PCR for enrichment of desired samples is performed with i7 primers together with primers that bind to selected i5, BCLIG, and BCRT indices. In this approach, the library may be designed to swap the BCRT and UMI location in library fragments, such that the nested PCR approach using BCRT retains the UMI sequence in resulting PCR products.

Third, a combined approach may be used. In a combined approach, a probe capture enrichment step is followed by i7-specific PCR enrichment step.

While these specific approaches use the design of the sci-RNA-seq3 libraries, the barcodes/indices used in other types of sc-libraries can also be exploited for enrichment steps. These sc-libraries include, BioRad-ddSEQ, 10X Genomics, InDrop, Drop-Seq, and Split-Seq. As shown in FIG. 4, the particular barcode structure of libraries (including the number of nucleotides in different barcode regions) can be used to design enrichment protocols. One skilled in the art could use information on various methods to design the most appropriate approach for enrichment based on the particular sc-library used for the initial sequencing.

### Example 2. Modified SCI-Seq Approach to Generate Library Fragments Comprising Contiguous Barcodes

A modified SCI-seq approach may be used to generate a single-cell RNA/DNA NGS library comprising contiguous barcodes, as shown in FIG. 5.

In a first step, tagmentation is performed with transposome complexes comprising a Tn5 transposase loaded with transposons comprising a BC1 sequence to incorporate a BC1 barcode. Cells or nuclei are distributed into reaction wells. If the starting target nucleic acid is RNA, cDNA synthesis is performed to generate the first and second strand. Tagmentation is performed with the well-specific barcodes (BC₁ barcodes). DNA is pooled from across wells. Gap repair is performed (3′ fill-in), followed by 5′ phosphorylation and generation of 3′A tail ends.

In a second step, T/A ligation is performed with one or more barcodes (BC2, . . . , BCX). These barcodes may be nonrandom. For this step, nuclei or cell are re-distributed into reaction wells, followed by T-tailed adapter ligation with well-specific barcodes (BC2 barcodes). DNA was pooled from across wells, followed by 5′ phosphorylation and generation of 3′ A-tail. Alternatively, library fragments may have a C/G overhang for subsequent C/G-ligation (used for every other barcoding round). These steps are repeated in multiple barcoding rounds, as necessary.

In a third step, T/A ligation is performed to generate the desired fragments with BCn barcodes. For this step, nuclei or cells are re-distributed into reaction wells, and T-tailed Y-shaped adapters are ligated with well-specific barcodes. Then DNA was pooled from across wells, and PCR was performed with sample indices.

During sc-library generation, the library does not have to be fully constructed. Stubby asymmetric ends can improve the specificity of hybridization and/or PCR results.

The resulting library can then be used for an initial sequencing, followed by enrichment or depletion based on the contiguous barcodes present in library fragments. The presence of contiguous barcodes may improve later enrichment by PCR, as primers can be designed across the full contiguous barcodes.

### Example 3. Method for Use with Distributed Microbial Cells in a Metagenomics Sample

The present methods may be used for metagenomics, such as organism genome assembly, wherein the organisms are not cultivated. These organisms may be microbial cells, such as those in a sample taken from a patient.

For this method, cells are distributed into wells and tagmentation inserts BC1 (only). The DNA is pooled, followed by extension to blunt and generate A-tail. Samples are distributed to appropriate dilution of DNA.

Next, T/A ligations are performed with T-tailed adaptors comprising BC2. DNA is pooled and extension performed to blunt and generate A-tail. These steps are repeated to incorporate the desired number of barcodes (BCn).

For the last ligation, a forked adapter is added, followed by PCR to add i5/i7 and P5/P7 sequences. The P5 and P7 sequences are useful for methods of sequencing using Illumina platforms, although other sequences may be added if sequencing is performed on other platforms.

An initial sequencing reaction is performed followed by analysis. Analysis may include determination of abundant species/taxa from whole genome assembly or ribosomal DNA (rDNA) analysis. For example, the initial sequencing may be 16s rDNA (or rRNA) sequencing. Initial sequencing for rDNA or rRNA can reduce time and resources needed for this step, and these data may be sufficient to identify the abundant species or taxa.

Alternatively, if most of the microorganisms in a sample comprise intact single chromosomes or high molecular weight genomic DNA after extraction, contiguity preserving transposition sequencing (CPT-seq, Illumina) may be appropriate for sequencing. Use of CPT-seq and combinatorial indexing allows genome-wide haplotyping (See Amini et al., Nat Genet. 46(12): 1343-1349 (2014)). This approach can be applied to synthetic linked long-read libraries. Linked-long read libraries are (short-read) sequenced and the DNA barcode identifying exemplary parent ‘long’ molecule can be targeted for enrichment or depletion from the composite library, followed by secondary sequencing. For example, in working with metagenomic samples, prokaryotes have ˜1 chromosome and thus, linked long read sequencing methods such as CPT-seq can be useful for rare species characterization and resolved de novo assembly.

The initial sequencing can generate data on species/taxa of interest for enrichment or depletion. For example, specific probes or Cas9-guide RNAs can be designed against UBCs of abundant species taxa to allow their depletion for focus on rarer species/taxa of interest. The depletion of abundant species may be performed by hybrid capture or CRISPR digestion based on the barcodes associated with the abundant species.

After selection, the remaining library can be reamplified with universal primers (P5/P7). Then, resequencing can be performed.

If desired, multiple rounds of identification of abundant species/taxa can be performed, followed by another round of depletion. The identification and depletion processes can be repeated until sufficient depletion of the abundant species/taxa is seen in the sequencing data such that metagenomics characterization criteria are met.

If desired, whole genome sequencing may be performed for resequencing if the initial sequencing focused on rDNA or rRNA analysis. In such cases, the initial sequencing could be focused on ribosomal signal, while the final resequencing provides more comprehensive data on rarer species or taxa of interest.

### Example 4. NGS Library Construction with Physically Addressable Barcodes and Targeted Sequencing

Methods can also be used for generating physically addressable barcodes using a transposition reaction with a separate release step, as shown in FIG. 6.

Cells, nuclei, or HMW DNA are distributed into reaction wells. Cells or nuclei can then optionally be lysed to make DNA accessible for preparation. Transposition is performed with transposases loaded with a first barcode (Tn5-loaded with BC1). This step incorporates a tag with well-specific first barcodes, but the transposase is not released. The DNA can then be pooled from across wells. To accommodate a high cell throughput with a fixed 2-level barcoding scheme, the method may incorporate more barcodes per reaction wells.

The DNA is then redistributed into reaction wells and the transposase is released. Gap-filling (3′ extension) and 5′ phosphorylation are performed, and 3′A tail ends are added. A T-tailed Y-shaped adapter ligation with well-specific second barcodes (BC2) is performed. The DNA is pooled from across wells, and PCR is performed based on sample indices. The library does not have to be fully constructed at this step, as stubby asymmetric ends can improve specificity of hybridization of primer and/or PCR reaction.

### Example 5. Recombinase-Mediated Targeted Transposition

Sequence-specific transposition can be mediated by transposome complexes comprising recombinase-coated targeting oligonucleotides. As shown in FIG. 9, a sample comprising genomic DNA is combined with transposome complexes comprising the recombinase-coated targeting oligonucleotides.

The recombinase-coated oligonucleotides will “scan” along the double-stranded DNA (dsDNA) until a complementary sequence is found in the target DNA (white section of genomic DNA in FIG. 9). At this point, the recombinase will facilitate strand invasion to place this oligonucleotide into the dsDNA structure (via D-loop formation). This process will bring the transposome complex into close proximity to the targeted sequences, and subsequent transposition will insert the transposon sequences close to the site of strand invasion.

Targeted transposition via recombinase-loaded transposomes may be performed as follows. First, a first set of transposome oligonucleotides are annealed by combining 5 μl of 10X TEN buffer (100 mM Tris pH 8, 10 mM EDTA, 250 mM NaCl) with 17.5 μl of the oligonucleotide of SEQ ID NO: 1 and 27.5 μl of the oligonucleotide of SEQ ID NO: 2. The oligonucleotide of SEQ ID NO: 2 can be annealed (in a 3′ to 5′ orientation) to the oligonucleotide of SEQ ID NO: 1 by a process of heating to 95° C. for 10 minutes and then cooling to 10° C. at a 0.1° C./s ramp rate.

Similarly, a second set of annealed oligonucleotides can be generated by annealing the oligonucleotides of SEQ ID NOs: 3 and 4.

The annealed oligonucleotides can be loaded with the transposase Tn5 using the following protocol. 14.28 μl of 35 μM annealed oligonucleotides, 15.9 μl of 95.6 μM tsTn5 enzyme, and 220 μl of 50% glycerol storage buffer are combined and incubated overnight at 37° C. An additional 250 μl of 50% glycerol storage buffer can be added and stored at −20° C. until needed.

Next, the recombinase can be added to DNA, followed by tagmentation. The recombinase can be used to generate regions of single-stranded DNA via strand invasion to allow binding of oligonucleotide pairs. 10 μl of Tn5 loaded oligonucleotides “1” (annealed pair of SEQ ID NOs: 1 and 2) with 10 μl of Tn5 loaded oligonucleotides “2” (annealed pair of SEQ ID NOs: 3 and 4), 10 μl of 5× buffer (250 mM Tris pH7.6, 50 mM MgCl₂, 25 mM DTT, 2.5 mM ATP), 0.5 μg of DNA, 2 μl of 2 μg/μl RecA, and 17.5 μl H₂O (total volume 50 μl) can be combined, mixed gently, and incubated for 1 hour at 37° C.

The reaction can then be stopped by adding 10 μl of STOP buffer (1% SDS), vortexing for 1 minute at 1600 rpm, and incubating for 5 minutes at room temperature.

Size selection can be performed using 2.5×SPRI beads. 150 μl of SPRI beads is added to the tube and incubated 5 minutes at room temperature. A wash is performed 2 times using TWB wash buffer followed by removal of the TWB wash buffer.

Next, PCR library amplification is performed. 20 μl EPM mix (Illumina), 20 μl H₂O, and 10 μl P5-A14/P7-B15 primer mix (2 μM each primer in H₂O) are added to washed beads. The reaction is then placed onto a PCR machine programmed as follows: 68° C. for 3 minutes; 98° C. for 3 minutes; 8 cycles of 98° C. for 45 seconds, 62° C. for 30 seconds, and 68° C. for 2 minutes; 68° C. for 1 minute; and finally a hold at 4° C.

### Example 6. Targeted Transposition Using Single-Stranded Nucleic Acid and Targeting Oligonucleotides

Transposase can mediate transposition of double-stranded DNA, such as double-stranded DNA. Methods can be used to selectively generate regions of double-stranded DNA within a single-stranded target nucleic acid. This single-stranded nucleic acid may be generated by denaturing a double-stranded nucleic acid.

As shown in FIG. 10, targeting oligonucleotides can hybridize to sequences of interest within a single-stranded nucleic acid, such as when the targeting oligonucleotides are fully or partially complementary to the sequences of interest. In this embodiment, the targeting oligonucleotide does not require coating with a recombinase, and the targeting oligonucleotide does not have to be linked to the transposome in any way.

Regions of a single-stranded nucleic acid that are bound by a targeting oligonucleotide will now be double-stranded. When a transposome complex is added, it can then proceed to bind to the double-stranded regions and then generate tagged fragments. In other words, after hybridization of targeting oligonucleotides, standard transposomes can then be used and should only insert where the target DNA has been made double-stranded via hybridization. In this way, targeting oligonucleotides can be used to generate tagged fragments comprising specific regions of interest from a target nucleic acid.

A representative method of mediating tagmentation using targeting oligonucleotides is provided. 2 μl of oligonucleotides comprising SEQ ID NOs: 5 and 6 (100 μM stocks) are added to 500 ng of genomic DNA (such as PhiX). The reaction is diluted to a final volume of 50 μl in 1×TEN buffer (10 mM Tris pH8, 1 mM EDTA, 25 mM NaCl). The reaction is heated to 95° C. for 5 minutes to denature DNA and then cooled to 10° C. at a 0.1° C./s ramp rate.

Next, DNA is tagmented. 10 μl of Nextera Tn5 #1,10 μl of Nextera Tn5 #2, 10 μl of 5× tagmentation buffer, and 20 μl of annealed oligonucleotides+DNA from step above are combined. The reaction is incubated for 5 minutes at 41° C. followed by a hold at 10° C. The reaction is stopped by adding 10 μl of STOP buffer (1% SDS), vortexing for 1 minute at 1600 rpm, and incubating for 5 minutes at room temperature.

Size selection is performed using 2.5×SPRI beads. 150 μl of SPRI beads are added to the tube and incubated for 5 minutes at room temperature. The reaction is washed 2× using TWB wash buffer followed by removing TWB wash buffer.

Finally, PCR is used to amplify the library. 20 μl EPM mix (Illumina), 20 μl H₂O, and 10 μl P5-A14/P7-B15 primer mix (2 μM each primer in H₂O) is added. The reaction is placed onto a PCR machine programmed as follows: 68° C. for 3 minutes; 98° C. for 3 minutes; 8 cycles of 98° C. for 45 seconds, 62° C. for 30 seconds, and 68° C. for 2 minutes; 68° C. for 1 minute; and a hold at 4° C.

### Example 7. Targeted Transposition of Cell-Free DNA Using Zinc Finger DNA-Binding Domains

Sequence-specific transposition can also be performed with cfDNA, as outlined in FIG. 15. A plasma sample comprising cfDNA can be mixed with targeted transposome complexes comprising a zinc finger DNA-binding domain. The zinc finger DNA-binding domain may be comprised in a zinc finger nuclease (ZFN) as shown in FIG. 15, wherein the ZFN may be catalytically inactive. Further, the transposome complexes may be designed to allow immobilization to a solid support (such as with a first transposon comprising biotin at the 5′ end or a second transposon comprising biotin at the 3′ end).

The zinc finger DNA-binding domain can bind to specific DNA sequences of interest, such as those within or in close proximity to a gene that a user wants to sequence. This binding may occur while the cfDNA is bound to histones (i.e., without pre-treatment of the cfDNA with a protease). After tagmentation mediated by the targeted transposome complex, the targeted cfDNA library is bound to streptavidin beads. After gap-filling and ligation, the targeted library generated from the cfDNA can be released from solid support or amplified and/or sequenced on the solid support.

An advantage of this method versus other means of generating libraries from cfDNA is the ease of this method that avoids protease steps to remove histones before tagmentation. Any protease steps to remove histones from cfDNA would need to be followed by washing or other steps to remove the protease, because the protease would otherwise interfere with the transposase within the transposome complex. In this way, the method outlined in FIG. 15 provides improved ease and speed for the user.

Further, use of targeted transposomes can avoid a need for other types of enrichment steps. The zinc finger DNA-binding domain in the targeted transposome complex can specifically target to a sequence of interest. For example, targeted transposomes comprising zinc finger DNA-binding domains can generate libraries of fragments comprising sequences of genes known to be associated with inheritable diseases. In this way, cfDNA in the plasma of a pregnant patient can be used to generate a targeted library comprising the sequences of genes associated with inherited diseases to evaluate the potential presence of fetal mutations in the genes. Similarly, cfDNA from the plasma of a patient with cancer could be used to generate a targeted library comprising sequences of tumor suppressor genes and oncogenes to determine whether mutations associated with poor prognosis are present.

### Example 8. ShCAST (Scytonema hofmanni CRISPR Associated Transposase) Targeted Library Preparation and Enrichment

Targeted sequencing of specific genes using a separate enrichment step after library preparation may be time-consuming. For example, such a separate enrichment step may involve hybridizing oligonucleotide probes to library DNA and isolating the hybridized DNA on streptavidin-coated beads. Despite significant improvements in efficiency and time required, such separate enrichment protocols may take about two hours, and the multiple reagents and steps can make these protocols challenging to automate.

In comparison, methods disclosed herein may be used to prepare and enrich libraries for targeted sequencing of specific genes, using a single step for both preparation and enrichment. For example, FIGS. 16A-16B schematically illustrate example compositions and operations in a process for ShCAST (Scytonema hofmanni CRISPR associated transposase) targeted library preparation and enrichment. ShCAST includes Cas12k and a Tn7-like transposase that is capable of inserting DNA into specific sites in the E. coli genome using guide RNA (gRNA). These gRNA can be generated with affinity for one or more sequence of interest in a target nucleic acid using well-known design algorithms.

These methods can utilize ShCAST or a modified version of ShCAST incorporating a Tn5 transposase (ShCAST-Tn5) for targeted fragmentation and amplification of specific genes. As such, library preparation and enrichment steps are combined. A combined protocol simplifies and improves the efficiency of the target library sequencing workflow. A combined protocol can also reduce the number of steps and user touchpoints and thus facilitate automation.

In an exemplary method, gRNA may be designed to target specific genes (sequences of interest), and the spacing between the binding sites for the gRNAs within the target nucleic acid may be used to control the insert size. In other words, the gRNAs can be designed to bind to sequences within the target nucleic that result in targeting of transposome complexes to generate inserts (i.e., double-stranded DNA fragments) of a desired size. The gRNA and/or the ShCAST/ShCAST-Tn5 may be biotinylated. In a manner such as illustrated in FIG. 16A, gRNAs and transposable elements with adapters (e.g., Illumina adapters comprising sequences useful for amplification and/or sequencing methods) may be loaded into the transposase of ShCAST, resulting in complex 6000. In a manner such as illustrated in process flow 6010 of FIG. 16B, the resulting ShCAST/ShCAST-Tn5 complexes may be mixed with genomic DNA under fluidic conditions (e.g., low or no magnesium) that inhibit tagmentation, while allowing the complexes to bind to respective sequences in the target DNA. The complexes then may be isolated using streptavidin beads to which the biotinylated gRNA and/or ShCAST/ShCAST-Tn5 becomes coupled. Any unbound DNA may be washed away, e.g., to reduce or minimize off-target tagmentation. Then the fluidic conditions may be altered (e.g., sufficiently increasing magnesium) to promote tagmentation. A gap-fill-ligation step followed by heat dissociation may be used to release the library from beads in preparation for sequencing.

Note that in compositions and operations such as illustrated in FIGS. 16A-16B, the transposase portion of the complex may also be able to randomly insert into the DNA. Such insertion may be inhibited or minimized by mixing the ShCAST/ShCAST-Tn5 complexes with the genomic DNA under fluidic conditions (e.g., low or no magnesium) that inhibit tagmentation, thus allowing targets to be bound.

For further details regarding ShCAST, including the Cas12K and Tn7 therein, see Strecker et al., “RNA-Guided DNA insertion with CRISPR-associated transposases,” Science 365(6448): 48-53 (2019), the entire contents of which are incorporated by reference herein.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A targeted transposome complex comprising:
c. a transposase;
d. a first transposon comprising:
i. a 3′ transposon end sequence,
ii. a 5′ adaptor sequence, and
e. a catalytically inactive endonuclease associated with a guide RNA, wherein the guide RNA can direct endonuclease binding to one or more nucleic acid sequences of interest; and
f. a second transposon comprising the complement of the transposon end sequence.

2. The targeted transposome complex of claim 1, wherein the catalytically inactive endonuclease is from cyanobacteria Scytonema hofmanni (ShCAST), optionally wherein:
a. at least one of the gRNA and the transposase is biotinylated, and wherein at least one of the gRNA and transposase that is biotinylated is capable of coupling to a streptavidin-coated bead;
b. ShCAST comprises Cas12K;
c. the transposase comprises Tn5 or a Tn7-like transposase; and/or
d. the first transposon comprises at least one of a P5 adapter and a P7 adapter.

3. A targeted transposome complex comprising:
a. a transposase,
b. a first transposon comprising
i. a 3′ transposon end sequence;
ii. a 5′ adaptor sequence; and
c. a zinc finger DNA-binding domain, wherein the zinc finger DNA-binding domain can bind to one or more nucleic acid sequences of interest; and
d. a second transposon comprising the complement of the transposon end sequence.

4. The targeted transposome complex of claim 3, wherein:
(a) the zinc finger DNA-binding domain is comprised in a zinc finger nuclease, optionally wherein the zinc finger nuclease is catalytically inactive; and/or
(b) the one or more nucleic acid sequences of interest are comprised in DNA associated with histones, optionally wherein the DNA associated with histones is cell-free DNA.

5. A method of targeted generation of 5′ tagged fragments of a target nucleic acid comprising:
a. combining a sample comprising a double-stranded nucleic acid and a transposome complexes of claim 1 that is a targeted transposome complex; and
b. fragmenting the nucleic acid into a plurality of fragments by the transposase, by joining the 3′ end of the first transposon to the 5′ ends of the fragments to produce a plurality of 5′ tagged fragments.

6. A method of generating a library of tagged nucleic acid fragments comprising:
a. combining a sample comprising a double-stranded nucleic acid, a first transposome complex of claim 1 that is a targeted transposome complex, and a second transposome complex comprising a
i. transposase;
ii. a first transposon comprising a 3′ transposon end sequence and a 5′ adaptor sequence; and
iii. a second transposon comprising a 5′ transposon end sequence, wherein the 5′ transposon end sequence is complementary to the 3′ transposon end sequence; and
b. fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

7. A method of generating a library of tagged nucleic acid fragments comprising:
a. combining a sample comprising a double-stranded nucleic acid, a first transposome complex of claim 1 that is a targeted transposome complex, and a second transposome complex of claim 1 that is a targeted transposome complex; and
b. fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

8. The method of claim 5, wherein the combining a sample comprising a double-stranded nucleic acid with one or more transposome complex that is targeted comprises:
a. combining the sample with a zinc finger DNA-binding domain or a catalytically inactive endonuclease, wherein the zinc finger DNA-binding domain or catalytically inactive endonuclease is bound to a first binding partner, and
b. adding the transposase and first and second transposons, wherein the transposase is bound to a second binding partner, wherein the transposase can bind to the zinc finger DNA-binding domain or catalytically inactive endonuclease by pairing of the first and second binding partners.

9. A targeted transposome complex comprising:
a. a transposase,
b. a first transposon comprising
i. a 3′ transposon end sequence;
ii. a 5′ adaptor sequence; and
iii. a targeting oligonucleotide coated with a recombinase, wherein the targeting oligonucleotide can bind to one or more nucleic acid sequences of interest; and
c. a second transposon comprising a 5′ transposon end sequence, wherein the 5′ transposon end sequence is complementary to the 3′ transposon end sequence.

10. The transposome complex of claim 9, wherein the sequence of the targeting oligonucleotide is fully or partially complementary with the one or more nucleic acid sequences of interest and/or wherein the recombinase is UVSX, Rec233, or RecA.

11. A kit or composition comprising a first transposome complex of claim 9 that is a targeted transposome complex, and a second transposome complex comprising:
i. a transposase;
ii. a first transposon comprising a 3′ transposon end sequence and a 5′ adaptor sequence; and
iii. a second transposon comprising a 5′ transposon end sequence, wherein the 5′ transposon end sequence is complementary to the 3′ transposon end sequence.

12. A method of targeted generation of 5′ tagged fragments of a target nucleic acid comprising:
a. combining a sample comprising a double-stranded nucleic acid and a transposome complex of claim 9 that is a targeted transposome complex;
b. initiating strand invasion of the nucleic acid by the recombinase; and
c. fragmenting the nucleic acid into a plurality of fragments by the transposase, by joining the 3′ end of the first transposon to the 5′ ends of the fragments to produce a plurality of 5′ tagged fragments.

13. A method of generating a library of tagged nucleic acid fragments comprising:
a. combining a sample comprising a double-stranded nucleic acid, a first transposome complex of claim 9 that is a targeted transposome complex, and a second transposome complex comprising a
i. transposase;
ii. a first transposon comprising a 3′ transposon end sequence and a 5′ adaptor sequence; and
iii. a second transposon comprising a 5′ transposon end sequence, wherein the 5′ transposon end sequence is complementary to the 3′ transposon end sequence;
b. initiating strand invasion of the nucleic acid by the recombinase; and
c. fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

14. A method of generating a library of tagged nucleic acid fragments comprising:
a. combining a sample comprising a double-stranded nucleic acid, a first transposome complex of claim 9 that is a targeted transposome complex, and a second transposome complex of claim 9 that is a targeted transposome complex;
b. initiating strand invasion of the nucleic acid by the recombinase; and
c. fragmenting the nucleic acid into a plurality of fragments by the transposase, by joining the 3′ end of each first transposon to the 5′ ends of the target fragments to produce a plurality of first 5′ tagged target fragments generated from the first transposome complex and a plurality of second 5′ tagged target fragments generated from the second transposome complex.

15. The method of claim 14, wherein the targeting oligonucleotide comprised in the first transposome complex that is a targeted transposome complex and the second transposome complex that is a targeted transposome complex are different, optionally wherein the targeting oligonucleotide of the first transposome complex that is a targeted transposome complex and the second transposome complex that is a targeted transposome complex bind to opposite strands of the double-stranded nucleic acid.

16. The method of claim 12, wherein
(a) the temperature used for initiating strand invasion is below the optimum temperature for fragmenting by the transposase, optionally wherein initiating strand invasion is performed at 27° C. to 47° C. and/or wherein the fragmenting is performed at 45° C. to 65° C.; and/or
(b) a cofactor for the transposase is added to the transposome complexes after initiating invasion and before fragmenting.

17. A method of preserving contiguity information when sequencing a target nucleic acid comprising:
a. producing tagged fragments of the target nucleic acid according to the method of claim 12;
b. sequencing the 5′ tagged fragments or fully double-stranded tagged fragments to provide sequences of the fragments;
c. grouping sequences of fragments that comprise the sequence of the same targeting oligonucleotide; and
d. determining that a group of sequences were in proximity within the target nucleic acid if they comprise the sequence of the same targeting oligonucleotide.

18. A method of preserving contiguity information when sequencing a target nucleic acid comprising:
a. producing tagged fragments of the target nucleic acid according to the method of claim 12, wherein one or more adapter sequence comprises a unique molecular identifier (UMI) associated with a single targeting oligonucleotide sequence;
b. sequencing the 5′ tagged fragments or fully double-stranded tagged fragments to provide sequences of the fragments;
c. grouping sequences of fragments that comprise the sequence of the same UMI; and
d. determining that a group of sequences were in proximity within the target nucleic acid if they comprise the sequence of the same UMI.

19. A method of targeted generation of 5′ tagged fragments of nucleic acid comprising:
a. hybridizing one or more targeting oligonucleotides to a sample comprising single-stranded nucleic acid, wherein the one or more targeting oligonucleotides can each bind to a sequence of interest in the nucleic acid;
b. applying a transposome complex comprising:
i. a transposase;
ii. a first transposon comprising a 3′ transposon end sequence and a 5′ adaptor sequence; and
iii. a second transposon comprising a 5′ transposon end sequence, wherein the 5′ transposon end sequence is complementary to the 3′ transposon end sequence; and
c. fragmenting the nucleic acid into a plurality of fragments by the transposase, by joining the 3′ end of the first transposon to the 5′ ends of the fragments to produce a plurality of 5′ tagged fragments.

20. A method of characterizing desired samples in a mixed pool of samples comprising both desired samples and unwanted samples comprising:
a. to produce sequencing data from double-stranded nucleic acid, initially sequencing a library comprising a plurality of nucleic acid samples from the mixed pool, wherein each nucleic acid library comprises nucleic acids from a single sample and a unique sample barcode to distinguish the nucleic acids from the single sample from the nucleic acids from other samples in the library;
b. analyzing the sequencing data and identifying unique sample barcodes associated with sequencing data from desired samples;
c. performing a selection step on the library comprising:
i. enriching nucleic acid samples from desired samples and/or
ii. depleting nucleic acid samples from unwanted samples; and
d. resequencing the nucleic acid library.

21. The method of claim 20, wherein the mixed pool of samples comprises a mixed pool of cells, a mixed pool of nuclei, or a mixed pool of high molecular weight DNA and/or the unique sample barcode is a unique cellular barcode.

22. The method of any claim 20, wherein:
a. the enriching step comprises hybrid capture, capture via catalytically inactive endonucleases, or unique sample barcode-specific amplification; or
b. the depletion step comprises hybrid capture, capture via catalytically inactive endonucleases, CRISPR digestion, or cleavage by a complex comprising a ShCAST coupled to guide RNA (gRNA).

23. The method of claim 22, wherein the depletion step comprises cleavage by a complex comprising a ShCAST coupled to gRNA, optionally wherein:
a. the ShCAST comprises Cas12K;
b. the transposase comprises Tn5 or a Tn7-like transposase;
c. the nucleic acid samples from unwanted samples comprise double-stranded DNA; and/or
d. at least one of the gRNA and the transposase is biotinylated, wherein at least one of the gRNA and transposase that is biotinylated is capable of coupling to a streptavidin-coated bead.

24. The method of claim 20, wherein the endonuclease is associated with a guide RNA that binds to one or more unique sample barcode and/or guide RNAs are directed against unique sample barcodes associated with nucleic acids of unwanted samples or guide RNAs are directed against unique sample barcodes associated with nucleic acids of desired samples.

25. The method of claim 20, wherein
(a) the desired sample is a rare sample that is present in less than or equal to 1%, 0.1%, 0.01%, 0.001%, 0.0001%, 0.00001%, 0.000001%, 0.0000001%, 0.00000001%, or 0.000000001% of a mixed pool of samples;
(b) the method comprises a step of spatially separating the nucleic acid samples before incorporating a unique sample barcode and/or tagmentation prior to sequencing a plurality of nucleic acid samples from the mixed pool of samples; and/or
(c) a unique sample barcode is incorporated into each nucleic acid sample, optionally wherein the unique sample barcode is a single contiguous barcode or multiple discontiguous barcodes.

26. The method of claim 20, wherein the initial sequencing step:
a. does not comprise whole genome sequencing and the resequencing step comprises whole genome sequencing;
b. comprises targeted sequencing and the resequencing step comprises whole genome sequencing;
c. comprises targeted sequencing with one or more gene-specific primers, optionally wherein the gene-specific primer comprises a universal primer tail; and/or
d. comprises ribosomal sequencing and the resequencing step comprises whole genome sequencing.

27. The method of claim 20, wherein the method is used to sequence a microbe from an environmental sample, optionally wherein the method does not comprise culturing the microbe from the environmental sample.


SEQUENCE LISTING (8 sequences)

SEQ ID NO: 1 - DNA, 73 bp, synthetic construct
  misc_feature 1..73: note = 40-mer_A14_ME
  source 1..73: mol_type = other DNA
gccttttgtaataattaccgcagctcgcaggccaatttcgtcgtcggcagcgtcagatgtgtataagagacag

SEQ ID NO: 2 - DNA, 19 bp, synthetic construct
  misc_feature 1..19: note = ME' (3' to 5')
  source 1..19: mol_type = other DNA
tctacacatattctctgtc

SEQ ID NO: 3 - DNA, 84 bp, synthetic construct
  misc_feature 1..84: note = 40-mer_B15_ME
  source 1..84: mol_type = other DNA
tcaactttaccattattctgctggttagactggtcgttccttcggttctagtctcgtgggctcggagatgtgtataagagacag

SEQ ID NO: 4 - DNA, 19 bp, synthetic construct
  misc_feature 1..19: note = ME' (5' to 3')
  source 1..19: mol_type = other DNA
ctgtctcttatacacatct

SEQ ID NO: 5 - DNA, 30 bp, synthetic construct
  misc_feature 1..30: note = Single-stranded 30-mer DNA primer targeting PhiX DNA
  source 1..30: mol_type = other DNA
gccttttgtaataattaccgcagctcgcag

SEQ ID NO: 6 - DNA, 30 bp, synthetic construct
  misc_feature 1..30: note = Single-stranded 30-mer DNA primer targeting PhiX DNA
  source 1..30: mol_type = other DNA
ggcagaaagaggtaacgcagcaccggaacg

SEQ ID NO: 7 - DNA, 29 bp, synthetic construct
  misc_feature 1..29: note = P5 Primer
  modified_base 23: mod_base = OTHER; note = uracil
  source 1..29: mol_type = other DNA
aatgatacggcgaccaccgagatctacac

SEQ ID NO: 8 - DNA, 24 bp, synthetic construct
  misc_feature 1..24: note = P7 Primer
  source 1..24: mol_type = other DNA
caagcagaagacggcatacgagat